(19) United States
(12) Patent Application Publication
(10) Pub. No.: US 2026/0347797 A1
(43) Pub. Date: Nov. 5, 2026

(54) ENZYME CAPABLE OF DEHYDROXYLATING HYDROXYL GROUP IN UROLITHIN COMPOUND

(71) Applicant: DAICEL CORPORATION, Osaka (JP)

(72) Inventors: Jun OGAWA, Kyoto-shi (JP); Shigenobu KISHINO, Kyoto-shi (JP); Hiroaki YAMAMOTO, Tokyo (JP)

(73) Assignee: DAICEL CORPORATION, Osaka (JP)

(21) Appl. No.: 19/393,292

(22) Filed: Nov. 18, 2025

Related U.S. Application Data
(63) Continuation of application No. 17/629,694, filed on Jul. 22, 2020, now Pat. No. 12,497,599.

(30) Foreign Application Priority Data
Jul. 25, 2019 (JP) 2019-136904
Feb. 14, 2020 (JP) 2020-023399

(51) Int. Cl. C12N 9/02; C12P 17/06

(52) U.S. Cl. CPC C12N 9/0004; C12P 17/06

(57) ABSTRACT

An object of the present disclosure is, at least, to provide an enzyme that dehydroxylates hydroxyl groups at predetermined positions of urolithins having hydroxyl groups at the predetermined positions, and the object can be solved by an enzyme having the following properties (1) and (2): (1) dehydroxylating a hydroxyl group at the 4-position of urolithins; and (2) in the presence of methyl viologen (MV), being activated by one or more components selected from the group consisting of: reduced nicotinamide adenine dinucleotide (NADH); reduced nicotinamide adenine dinucleotide phosphate (NADPH); flavin adenine dinucleotide (FAD); and flavin adenine mononucleotide (FMN).

### CROSS-REFERENCE TO RELATED APPLICATIONS

This application is a Continuation of copending application Ser. No. 17/629,694, filed on Jan. 24, 2022, which is the National Phase under 35 U.S.C. § 371 of International Application No. PCT/JP2020/028541, filed on Jul. 22, 2020, which claims the benefit under 35 U.S.C. § 119(a) to Patent Application No. 2019-136904, filed in Japan on Jul. 25, 2019, and to Patent Application No. 2020-023399, filed in Japan on Feb. 14, 2020, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 10, 2026, is named “P201131WOUS-01 Sequence Listing.xml” and is 73,728 bytes in size. The sequence listing contained in this .XML file is part of the specification and is hereby incorporated by reference herein in its entirety.

### TECHNICAL FIELD

The present disclosure relates to an enzyme that dehydroxylates hydroxyl groups of urolithins.

### BACKGROUND ART

Urolithins such as urolithin A and urolithin C are known as intestinal metabolites of ellagic acid derived from, inter alia, ellagitannins included in berries such as pomegranates, raspberries, blackberries, cloudberries, strawberries, and walnuts.

For synthesizing these urolithins, a method has been reported in which 2-bromo-5-methoxybenzoic acid is used as a starting material and converted to 2-bromo-5-hydroxybenzoic acid through demethylation, which is then reacted with resorcinol to form urolithin A (Non-Patent Literature 1).

However, this type of chemical synthesis is not suitable in use involving urolithins as part of the materials for functional food products, including beverages and supplements.

It is known that, after ellagitannins and ellagic acids are consumed, those are metabolized by intestinal microflora and converted into urolithins.

In recent years, a microorganism belonging to the species Gordonibacter urolithinfaciens and a microorganism belonging to the species Gordonibacter pamelaeae have been discovered as enterobacteria that produce urolithin C, which is a type of urolithin, from ellagic acid, and a method has been reported in which these enterobacteria are used to produce urolithin C through fermentation of ellagic acid (Patent Document 1 and Non-Patent Literature 1).

### PRIOR ART DOCUMENTS

### Patent Document

Patent Document 1: WO 2014/147280

### Non-Patent Literature

Non-Patent Literature 1: J. Agric. Food Chem., 56, 393-400 (2008)

### SUMMARY OF THE INVENTION

### Problems to be Solved by the Invention

An object of the present disclosure is, at least, to provide an enzyme that dehydroxylates hydroxyl groups at predetermined positions of urolithins having hydroxyl groups at the predetermined positions.

### Means for Solving the Problems

Through proteome analysis of the specific strain of Gordonibacter urolithinfaciens, the present inventors discovered that, when the microorganisms are cultured in a culture medium containing ellagic acid, proteins with significantly high expression levels will be obtained, in contrast to a case where the microorganisms are cultured in a culture medium that does not contain ellagic acid.

The present inventors also purified an enzyme that catalyzes a reaction in which the hydroxyl group at the 4-position of urolithins having a hydroxyl group at the 4-position is dehydroxylated from the predetermined strain, and then subjected the enzyme to proteome analysis, and the present inventors found the result that the purified enzyme that was obtained matched some of the proteins of which expression level increased significantly when the microorganism were cultured in a culture medium containing ellagic acid. The present inventors then introduced a gene that would encode for the enzyme thereof into different types of microorganism, and thereby successfully expressed the enzyme and identified the function of the gene.

It was also discovered that in the vicinity of the genome of the gene encoding the enzyme, a gene that encodes an enzyme having homology with the abovementioned gene and that catalyzes a reaction of dehydroxylating the 10-position hydroxyl group of urolithins having a hydroxyl group at the 10-position, as well as a gene that encodes ellagic acid lactonase, which hydrolyzes lactones of ellagic acid to produce urolithin M5, and a gene that encodes a urolithin transporter are present.

The present disclosure includes the following aspects.

<1> An enzyme having the following properties (1) and (2):
(1) dehydroxylating a hydroxyl group at the 4-position of urolithins; and
(2) in the presence of methyl viologen (MV), being activated by one or more components selected from the group consisting of: reduced nicotinamide adenine dinucleotide (NADH); reduced nicotinamide adenine dinucleotide phosphate (NADPH); flavin adenine dinucleotide (FAD); and flavin adenine mononucleotide (FMN).

<2> The enzyme according to <1>, having the following properties (3) and (4):
(3) having an optimum pH of 5.5 or higher and 7.5 or less; and
(4) having a SDS-PAGE result that includes a band indicating a molecular weight of 81,000 or greater and 99,000 or less.

<3> The enzyme according to <1> or <2>, having the following property (5):
(5) having an optimum temperature of 37 °C or higher and 50 °C or lower.

<4> The enzyme according to any one of <1> to <3>, being derived from a microorganism belonging to the genus Gordonibacter.

<5> The enzyme according to <4>, wherein the microorganism belonging to the genus Gordonibacter is one or more selected from the group consisting of: a microorganism belonging to Gordonibacter urolithinfaciens; a microorganism belonging to Gordonibacter pamelaeae; and a microorganism belonging to Gordonibacter faecihominis.

<6> The enzyme according to any one of <1> to <5>, comprising:
an amino acid sequence represented by SEQ ID NO: 1, and an amino acid sequence represented by SEQ ID NO: 2; or
an amino acid sequence represented by SEQ ID NO: 13, and an amino acid sequence represented by SEQ ID NO: 14.

<7> The enzyme according to any one of <1> to <6>, comprising:
an amino acid sequence represented by SEQ ID NO: 1, an amino acid sequence represented by SEQ ID NO: 2, and an amino acid sequence represented by SEQ ID NO: 3; or
an amino acid sequence represented by SEQ ID NO: 13, an amino acid sequence represented by SEQ ID NO: 14, and an amino acid sequence represented by SEQ ID NO: 15.

<8> A polynucleotide comprising:
a base sequence represented by SEQ ID NO: 7, and a base sequence represented by SEQ ID NO: 8; or
a base sequence represented by SEQ ID NO: 19, and a base sequence represented by SEQ ID NO: 20.

<9> A polynucleotide comprising:
a base sequence represented by SEQ ID NO: 7, a base sequence represented by SEQ ID NO: 8, and a base sequence represented by SEQ ID NO: 9; or
a base sequence represented by SEQ ID NO: 19, a base sequence represented by SEQ ID NO: 20, and a base sequence represented by SEQ ID NO: 21.

<10> A recombinant vector comprising the polynucleotide according to <8> or <9>.

<11> A transformant, in which the polynucleotide according to <8> or <9> is expressively retained, or the vector according to <10> is expressively retained.

<12> The transformant according to <11>, of which host is a microorganism belonging to the genus Rhodococcus.

<13> A method for producing a protein encoded by the polynucleotide according to <8> or <9>, the method comprising culturing the transformant according to <11> or <12>.

<14> A method for dehydroxylating a hydroxyl group at the 4-position of urolithins, the method comprising the following Step (I):
Step (I): bringing one or more components selected from the following (i) to (iv) into contact with urolithins having a hydroxyl group at the 4-position, so as to dehydroxylate the hydroxyl group at the 4-position:
(i) the enzyme according to any one of <1> to <7>;
(ii) a protein encoded by the polynucleotide according to <8> or <9>;
(iii) a microorganism that produces the enzyme according to (i) above or the protein according to (ii) above; and
(iv) a treated product of the microorganism according to (iii) above.

<15> The method according to <14>, wherein the urolithins are urolithin M5, urolithin D, or urolithin E; and products produced by the dehydroxylation of the hydroxyl group at the 4-position of the urolithins are urolithin M6, urolithin C, and urolithin M7, respectively.

<16> An enzyme having the following properties (1) and (2):
(1) dehydroxylating a hydroxyl group at the 10-position of urolithins; and
(2) in the presence of methyl viologen (MV), being activated by one or more components selected from the group consisting of: reduced nicotinamide adenine dinucleotide (NADH); reduced nicotinamide adenine dinucleotide phosphate (NADPH); flavin adenine dinucleotide (FAD); and flavin adenine mononucleotide (FMN).

<17> The enzyme according to <16>, having the following properties (3) and (4):
(3) having an optimum pH of 5.0 or higher and 7.0 or less; and
(4) having an optimum temperature of 37 °C or higher and 42 °C or lower.

<18> The enzyme according to <16> or <17>, being derived from a microorganism belonging to the genus Gordonibacter.

<19> The enzyme according to <18>, wherein the microorganism belonging to the genus Gordonibacter is one or more selected from the group consisting of: a microorganism belonging to Gordonibacter urolithinfaciens; a microorganism belonging to Gordonibacter pamelaeae; and a microorganism belonging to Gordonibacter faecihominis.

<20> The enzyme according to any one of <16> to <19>, comprising:
an amino acid sequence represented by SEQ ID NO: 4, and an amino acid sequence represented by SEQ ID NO: 5; or
an amino acid sequence represented by SEQ ID NO: 16, and an amino acid sequence represented by SEQ ID NO: 17.

<21> The enzyme according to any one of <16> to <20>, comprising:
an amino acid sequence represented by SEQ ID NO: 4; an amino acid sequence represented by SEQ ID NO: 5; and an amino acid sequence represented by SEQ ID NO: 6; or
an amino acid sequence represented by SEQ ID NO: 16; an amino acid sequence represented by SEQ ID NO: 17; and an amino acid sequence represented by SEQ ID NO: 18.

<22> A polynucleotide comprising:
a base sequence represented by SEQ ID NO: 10, and a base sequence represented by SEQ ID NO: 11; or
a base sequence represented by SEQ ID NO: 22, and a base sequence represented by SEQ ID NO: 23.

<23> The polynucleotide according to <22>, comprising:
a base sequence represented by SEQ ID NO: 10, a base sequence represented by SEQ ID NO: 11, and a base sequence represented by SEQ ID NO: 12; or
a base sequence represented by SEQ ID NO: 22, a base sequence represented by SEQ ID NO: 23, and a base sequence represented by SEQ ID NO: 24.

<24> A recombinant vector comprising the polynucleotide according to <22> or <23>.

<25> A transformant, in which the polynucleotide according to <22> or <23> is expressively retained, or the vector according to <24> is expressively retained.

<26> The transformant according to <25>, of which host is a microorganism belonging to the genus Rhodococcus.

<27> A method for producing a protein encoded by the polynucleotide according to <22> or <23>, the method comprising culturing the transformant according to <25> or <26>.

<28> A method for producing the enzyme according to any one of <16> to <21>, the method comprising culturing the microorganism belonging to the genus Gordonibacter.

<29> The method according to <28>, wherein the microorganism belonging to the genus Gordonibacter is one or more selected from the group consisting of: a microorganism belonging to Gordonibacter urolithinfaciens; a microorganism belonging to Gordonibacter pamelaeae; and a microorganism belonging to Gordonibacter faecihominis.

<30> A method for dehydroxylating a hydroxyl group at the 10-position of urolithins, the method comprising the following Step (I):
Step (I): bringing one or more components selected from the following (i) to (iv) into contact with urolithins having a hydroxyl group at the 10-position, so as to dehydroxylate the hydroxyl group at the 10-position;
(i) the enzyme according to any one of <16> to <21>;
(ii) a protein encoded by the polynucleotide according to <22> or <23>;
(iii) a microorganism that produces the enzyme according to (i) above or the protein according to (ii) above; and
(iv) a treated product of the microorganism according to (iii) above.

<31> The method according to <30>, wherein the urolithins are urolithin M5, urolithin M6, or urolithin M7; and products produced by the dehydroxylation of the hydroxyl group at the 10-position of the urolithins are urolithin D, urolithin C, and urolithin A, respectively.

<32> A method for producing urolithin C, the method comprising the following Steps (I) to (III):

Step (I): allowing a microorganism having an ability to produce urolithin M5 from ellagic acid to produce urolithin M5 from ellagic acid;

Step (II): bringing one or more components selected from the following (i) to (iv) into contact with the urolithin M5, so as to produce urolithin M6:
(i) the enzyme according to any one of <1> to <7>;
(ii) a protein encoded by the polynucleotide according to <8> or <9>;
(iii) a microorganism that produces the enzyme according to (i) above or the protein according to (ii) above; and
(iv) a treated product of the microorganism according to (iii) above; and
Step (III): bringing one or more components selected from the following (v) to (viii) into contact with the urolithin M6, so as to produce urolithin C:
(v) the enzyme according to any one of <16> to <21>;
(vi) a protein encoded by the polynucleotide according to <22> or <23>;
(vii) a microorganism that produces the enzyme according to (v) above or the protein according to (vi) above; and
(viii) a treated product of the microorganism according to (vii) above.

<33> A method for producing urolithin C, the method comprising the following Steps (I) to (III):
Step (I): allowing a microorganism having an ability to produce urolithin M5 from ellagic acid to produce urolithin M5 from ellagic acid;
Step (II): bringing one or more components selected from the following (i) to (iv) into contact with the urolithin M5, so as to produce urolithin D:
(i) the enzyme according to any one of <16> to <21>;
(ii) a protein encoded by the polynucleotide according to <22> or <23>;
(iii) a microorganism that produces the enzyme according to (i) above or the protein according to (ii) above; and
(iv) a treated product of the microorganism according to (iii) above; and
Step (III): bringing one or more components selected from the following (v) to (viii) into contact with the urolithin D, so as to produce urolithin C:
(v) the enzyme according to any one of <1> to <7>;
(vi) a protein encoded by the polynucleotide according to <8> or <9>;
(vii) a microorganism that produces the enzyme according to (v) above or the protein according to (vi) above; and
(viii) a treated product of the microorganism according to (vii) above.

<34> A method for producing urolithin A, the method comprising the following Step (I):
Step (I): allowing a microorganism having an ability to produce urolithin A from urolithin C to produce urolithin A from the urolithin C produced by the production method according to <32> or <33>.

<35> A protein, comprising:
an amino acid sequence represented by SEQ ID NO: 25; or an amino acid sequence represented by SEQ ID NO: 27.

<36> A polynucleotide, comprising:
a base sequence represented by SEQ ID NO: 26; or a base sequence represented by SEQ ID NO: 28.

<37> A recombinant vector comprising the polynucleotide according to <36>.

<38> A transformant, in which the polynucleotide according to <36> is expressively retained, or the vector according to <37> is expressively retained.

<39> A method for producing a protein encoded by the polynucleotide according to <36>, the method comprising culturing the transformant according to <38>.

<40> A method for promoting uptake of ellagic acid into a cell of the transformant according to <38> from outside the cell, the method comprising the following Step (I):
Step (I): bringing the transformant according to <38> into contact with ellagic acid.

<41> A method for producing urolithin M5, the method comprising the following Step (I):
Step (I): bringing the transformant according to <38>, wherein host of the transformant is a microorganism having an ability to produce urolithin M5 from ellagic acid, into contact with ellagic acid, so as to produce urolithin M5 from the ellagic acid.

<42> A method for producing urolithin C, the method comprising the following Step (I):
Step (I): bringing the transformant according to <38>, wherein host of the transformant is a microorganism having an ability to produce urolithin C from ellagic acid, into contact with ellagic acid, so as to produce urolithin C from the ellagic acid.

<43> A method for producing urolithin A, the method comprising the following Step (I) and Step (II):
Step (I): bringing the transformant according to <38>, wherein host of the transformant is a microorganism having an ability to produce urolithin C from ellagic acid, into contact with ellagic acid, so as to produce urolithin C from the ellagic acid; and
Step (II): allowing a microorganism having an ability to produce urolithin A from urolithin C to produce urolithin A from the urolithin C.

<44> A protein, comprising
an amino acid sequence represented by SEQ ID NO: 29; or
an amino acid sequence represented by SEQ ID NO: 31.

<45> A polynucleotide, comprising
a base sequence represented by SEQ ID NO: 30; or
a base sequence represented by SEQ ID NO: 32.

<46> A recombinant vector comprising the polynucleotide according to <45>.

<47> A transformant, in which the polynucleotide according to <45> is expressively retained, or the vector according to <46> is expressively retained.

<48> A method for producing a protein encoded by the polynucleotide according to <45>, the method comprising culturing the transformant according to <47>.

<49> A method for producing urolithin M5, the method comprising the following Step (I):
Step (I): bringing the transformant according to <47> into contact with ellagic acid, so as to produce urolithin M5 from the ellagic acid.

<51> A method for producing urolithin A, the method comprising the following Step (I) and Step (II):
Step (I): bringing the transformant according to <47>, wherein host of the transformant is a microorganism having an ability to produce urolithin C from urolithin M5, into contact with ellagic acid, so as to produce urolithin M5 from the ellagic acid, and producing urolithin C from the urolithin M5; and
Step (II): allowing a microorganism having an ability to produce urolithin A from urolithin C to produce urolithin A from the urolithin C.

### Effect of the Invention

The present disclosure, at least, achieves an effect of providing an enzyme that dehydroxylates hydroxyl groups at predetermined positions of urolithins having hydroxyl groups at the predetermined positions.

### BRIEF DESCRIPTION OF THE DRAWINGS

FIG. 1 (photograph as a substitute of drawing) illustrates the results obtained in one experimental example of the present disclosure through Native-PAGE of an enzyme that dehydroxylates a hydroxyl group at the 4-position of urolithins and of an enzyme that dehydroxylates the hydroxyl group at the 10-position of the urolithins.

FIG. 2 (photograph as a substitute of drawing) illustrates the results obtained in one experimental example of the present disclosure through SDS-PAGE of an enzyme that dehydroxylates a hydroxyl group at the 4-position of urolithins and of an enzyme that dehydroxylates the hydroxyl group at the 10-position of the urolithins.

FIG. 3 is a graph illustrating the temperature dependency of an enzyme that dehydroxylates the hydroxyl group at the 4-position of urolithins in one experimental example of the present disclosure.

FIG. 4 is a graph illustrating the pH dependency of enzymes that dehydroxylate the hydroxyl group at the 4-position of urolithins in one experimental example of the present disclosure.

FIG. 5 is a graph illustrating the temperature stability of an enzyme that dehydroxylates the hydroxyl group at the 4-position of urolithins in one experimental example of the present disclosure.

FIG. 6 is a graph illustrating the pH stabilities of enzymes that dehydroxylate the hydroxyl group at the 4-position of urolithins in one experimental example of the present disclosure.

FIG. 7 is a graph illustrating the dehydroxylation activity of bacterial cells that dehydroxylate the hydroxyl group at the 10-position of urolithins in one experimental example of the present disclosure.

FIG. 8 is a graph illustrating the temperature dependency of an enzyme that dehydroxylates the hydroxyl group at the 10-position of urolithins in one experimental example of the present disclosure.

FIG. 9 is a graph illustrating the pH dependency of enzymes that dehydroxylate the hydroxyl group at the 10-position of urolithins in one experimental example of the present disclosure.

### MODE FOR CARRYING OUT THE INVENTION

Hereinafter, the present invention will be described in detail.

Note that each of the configurations, combinations thereof, and the like in each aspect are examples, and additions, omissions, substitutions, and other changes of the configuration may be made as appropriate within a scope that does not depart from the spirit of the present invention. The present disclosure is not limited by the aspects and is limited only by the scope of the claims.

Each aspect disclosed in the present specification can be combined with any other feature disclosed herein.

Note that in the present disclosure, the depository number of strains beginning with the wording DSM is the number assigned to the microorganisms stored by Deutsche Sammlung von Mikroorganismen und Zellkulturen GmbH (DSMZ).

Also, the depository number of strains beginning with the wording JCM is the number assigned to the microorganism stored by the RIKEN BioResource Research Center.

Urolithins are represented by the following General Formula (1):

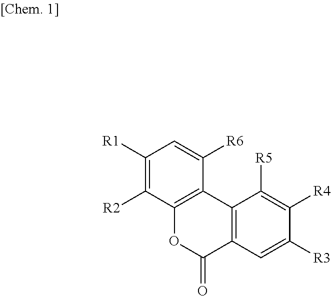

where in General Formula (1), R1 to R6 each independently represent a hydroxyl group, a hydrogen atom, or a methoxy group, and one or more of R1 to R6 are hydroxyl groups.

The hydroxyl group at the 4-position of the urolithins described in the present specification refers to the hydroxyl group of urolithins in which R2 in General Formula (1) above is a hydroxyl group, and the hydroxyl group at the 10-position refers to the hydroxyl group of the urolithins in which R5 in General Formula (1) above is a hydroxyl group.

<1-1-1 (A). Enzyme that Dehydroxylates the Hydroxyl Group at the 4-Position of Urolithins>

One aspect of the present disclosure is an enzyme that dehydroxylates the hydroxyl group at the 4-position of urolithins. More specifically, the enzyme is an enzyme that catalyzes a reaction in which the hydroxyl group at the 4-position of urolithins having a hydroxyl group at the 4-position is dehydroxylated. It is speculated that the enzyme thereof is constituted from one sub-unit, two sub-units, or three sub-units.

An enzyme according to the present aspect is an enzyme having the properties (1) and (2) below.

(1) The enzyme dehydroxylates a hydroxyl group at the 4-position of urolithins.

(2) The enzyme is, in the presence of methyl viologen (MV), activated by one or more components selected from the group consisting of: reduced nicotinamide adenine dinucleotide (NADH); reduced nicotinamide adenine dinucleotide phosphate (NADPH); flavin adenine dinucleotide (FAD); and flavin adenine mononucleotide (FMN).

The urolithins in the present aspect are preferably urolithin M5, urolithin D, or urolithin E. When the hydroxyl group at the 4-position of each of these urolithins is dehydroxylated by the enzyme according to the present aspect, urolithin M6, urolithin C, and urolithin M7 are respectively produced.

The enzyme according to the present aspect is activated by a cofactor. Examples of the cofactor include methyl viologen (MV), reduced nicotinamide adenine dinucleotide (NADH), reduced nicotinamide adenine dinucleotide phosphate (NADPH), flavin adenine dinucleotide (FAD), and flavin adenine mononucleotide (FMN).

With the cofactor of the present aspect, the activity of dehydroxylation of the hydroxyl group at the 4-position of the urolithins is increased, and therefore MV and NADH are preferably used, MV and NADPH are more preferably used, MV, NADPH, and FMN are even more preferably used, and MV, NADPH, and FAD are yet even more preferably used.

The enzyme according to the present aspect preferably has the properties (3) and (4) below.

(3) The optimum pH is preferably 5.5 or higher, and more preferably 6.0 or higher, and is preferably 7.5 or less, more preferably 7.0 or less, and even more preferably 6.5 or less.

(4) The SDS-PAGE result includes a band indicating a molecular weight of preferably 81,000 or greater, more preferably 85,000 or greater, and even more preferably 87,000 or greater, and also preferably 99,000 or less, more preferably 95,000 or less, and even more preferably 93,000 or less.

The enzyme according to the present aspect preferably has the property of (5) below.

(5) The optimum temperature is preferably 37 °C or higher, more preferably 38 °C or higher, and even more preferably 40 °C or higher, and is also preferably 50 °C or lower, more preferably 46 °C or lower, and even more preferably 44 °C or lower.

The enzyme according to the present aspect is preferably derived from a microorganism belonging to the genus Gordonibacter.

Such an enzyme is more preferably one or more selected from the group consisting of: a microorganism belonging to Gordonibacter urolithinfaciens; a microorganism belonging to Gordonibacter pamelaeae; and a microorganism belonging to Gordonibacter faecihominis.

The microorganism belonging to Gordonibacter urolithinfaciens is preferably of the Gordonibacter urolithinfaciens DSM 27213 strain.

The microorganism belonging to Gordonibacter pamelaeae is preferably of the DSM 19378 strain.

The microorganism belonging to Gordonibacter faecihominis is preferably of the JCM 16058 strain.

The Gordonibacter urolithinfaciens DSM 27213 strain is not limited to the same strain thereof, and may be bacteria substantially equivalent to the deposited strain. Substantially equivalent bacteria are microorganisms that belong to Gordonibacter urolithinfaciens and can exhibit an effect according to the present aspect, such as expressing an enzyme according to the present aspect; and furthermore, are microorganisms in which the base sequence of the 16S rRNA gene thereof has homology of preferably 98% or higher, more preferably 99% or higher, and even more 100% in relation to the base sequence of the 16S rRNA gene of the deposited strain, and the microorganisms thereof preferably have the same mycological properties as the deposited strain. Moreover, as long as the effect of the present aspect is not impaired, the strain may be a strain that is bred from the deposited strain or a strain substantially equivalent thereto, by a mutation treatment, genetic recombination, selection of a natural mutant strain, or the like.

This also applies to the Gordonibacter pamelaeae DSM 19378 strain and the Gordonibacter faecihominis JCM 16058 strain.

Microorganisms belonging to the genus Gordonibacter are cultured in a general culture medium that is used in culturing anaerobes, and examples of such culture medium includes Anaerobe Basal Broth (CM0957, available from ThermoFisher Scientific), Wilkins-Chalgren Anaerobe Broth (CM0643, available from ThermoFisher Scientific), GAM culture medium (available from Nissui Pharmaceutical Co., Ltd.), and a modified GAM culture medium (available from Nissui Pharmaceutical Co., Ltd.).

The culturing temperature is preferably 25 °C or higher, more preferably 30 °C or higher, and even more preferably 33 °C or higher, and is also preferably 45 °C or lower, more preferably 42 °C or lower, and even more preferably 40 °C or lower.

Furthermore, a water soluble organic material can be added as a carbon source, for example. Examples of the water soluble organic material include saccharides, such as sorbose, fructose, glucose, dextrin, and soluble starch; alcohols such as methanol; organic acids such as valeric acid, butyric acid, propionic acid, acetic acid, formic acid, and succinic acid; and amino acids such as arginine, methionine, phenylalanine, valine, and glutamic acid.

The concentration of organic material added as a carbon source in the culture medium can be adjusted, as appropriate, to facilitate efficient growth. Typically, the concentration can be selected in a range of from 0.1 to 10 wt/vol %.

In addition to the carbon source described above, a nitrogen source may be added to the culture medium. Various nitrogen compounds that may be used ordinarily in cultivation or fermentation can be used as the nitrogen source.

Examples of preferred inorganic nitrogen sources include ammonium salts and nitrates. More preferable examples of inorganic nitrogen sources include ammonium sulfate, ammonium chloride, ammonium phosphate, ammonium hydrogen phosphate, triammonium citrate, potassium nitrate and sodium nitrate.

On the other hand, examples of preferred organic nitrogen sources include amino acids, yeast extracts, peptones (such as milk-derived peptones, soybean-derived peptones, and soybean-derived peptides), meat extracts (for example, Lab Lemco powder, bonito extract, tuna extract, bonito extract, bouillon, and shellfish), liver extract, and digestive serum powder. Examples of more preferable organic nitrogen sources include arginine, cysteine, citrulline, lysine, tryptophan, yeast extract, and peptones.

Furthermore, in addition to the carbon source or nitrogen source, microbial growth factors such as extracts, vitamins, and metal salts and inorganic compounds can also be added to the culture medium. Examples of extracts include hemin, heme iron, digestive serum powder, liver extract, and blood digestion products. Examples of vitamins include biotin, folic acid, pyridoxal, thiamine, riboflavin, nicotinic acid, nicotinamide, pantothenic acid, vitamin B12, thioctic acid, p-aminobenzoic acid, and vitamin K. Examples of metal salts and inorganic compounds include potassium dihydrogen phosphate, magnesium sulfate, manganese sulfate, sodium chloride, cobalt chloride, calcium chloride, zinc sulfate, copper sulfate, alum, sodium molybdate, potassium chloride, boric acid, and the like, nickel chloride, sodium tungstate, sodium selenate, sodium selenite, ammonium iron(II) sulfate, iron(II) citrate, sodium acetate trihydrate, magnesium sulfate heptahydrate, and manganese sulfate tetrahydrate. These metals may also be added in the form of mineral yeast.

Methods for producing a culture solution by adding plant- and/or animal-derived growth cofactors such as these inorganic compounds and vitamins are well known. The culture medium can be a liquid, a semi-solid, or a solid. A preferred form of the culture medium is a liquid culture medium.

Production of an enzyme according to the present aspect using the microorganisms belonging to the genus Gordonibacter is induced by adding ellagic acid or a precursor of ellagic acid, which is a raw material (substrate), to the culture medium. Examples of precursors of ellagic acid include ellagitannins such as punicalagin and geraniin. The raw material (substrate) is preferably added at an amount at which the concentration in the culture medium is from 0.01 g/L to 20 g/L.

In order to recover an enzyme according to the present aspect, the enzyme being produced by the microorganisms belonging to the genus Gordonibacter, the culture is recovered after production of the enzyme according to the present aspect, and in a buffer solution to which has been added a reducing agent such as cysteine, 2-mercaptoethanol, or dithiothreitol, or a protease inhibitor such as phenylmethane sulfonyl fluoride (PMFS), pepstatin A, or ethylenediamine tetra-acetic acid, the microorganisms are crushed to obtain a cell-free extract, and the enzyme can then be purified from the cell-free extract by combining, as appropriate, fractions according to protein solubility, and carrying out various types of chromatography. These may all be carried out according to routine methods.

The one sub-unit, two sub-units, or three sub-units of the Gordonibacter urolithinfaciens DSM 27213 strain, the sub-units thereof being presumed to constitute the enzyme according to the present aspect, are preferably selected from combinations of GuUroA1, GuUroA2, and GuUroA3.

The amino acid sequences of GuUroA1, GuUroA2, and GuUroA3 are the sequences represented by SEQ ID NOS: 1 to 3, respectively.

The base sequences of the genes encoding GuUroA1, GuUroA2, and GuUroA3 are sequences represented by SEQ ID NOS: 7 to 9, respectively.

Moreover, as previously mentioned, the enzyme according to the present aspect preferably has the property of (4). The molecular weight of from 81,000 to 99,000 is presumed to be the molecular weight of the GuUroA1 sub-unit.

The one sub-unit, the two sub-units, or the three sub-units of the Gordonibacter pamelaeae DSM 19378 strain, the sub-units thereof being presumed to constitute an enzyme according to the present aspect, are preferably selected from combinations of GpUroA1, GpUroA2 and GpUroA3. The amino acid sequences of GpUroA1, GpUroA2, and GpUroA3 are the sequences represented by SEQ ID NOS: 13 to 15, respectively.

The base sequences of the genes encoding GpUroA1, GpUroA2, and GpUroA3 are the sequences represented by SEQ ID NOS: 19 to 21, respectively.

Accordingly, the enzyme according to the present aspect is preferably an enzyme containing:
one or more amino acid sequence selected from the group consisting of the amino acid sequence represented by SEQ ID NO: 1, the amino acid sequence represented by SEQ ID NO: 2, and the amino acid sequence represented by SEQ ID NO: 3; or
one or more amino acid sequence selected from the group consisting of the amino acid sequence represented by SEQ ID NO: 13, the amino acid sequence represented by SEQ ID NO: 14, and the amino acid sequence represented by SEQ ID NO: 15.

Also, as long as GuUroA1 exhibits, as one sub-unit itself, or when constituting a unit along with GuUroA2 and/or GuUroA3, which are other sub-units, activity to dehydroxylate the hydroxyl group at the 4-position of urolithins, the GuUroA1 may be a protein formed from amino acids, the protein thereof being such that in the amino acid sequence represented by SEQ ID NO: 1, one to a plurality of amino acids are substituted or deleted, or one to a plurality of amino acids are inserted or added. One to a plurality means preferably from 1 to 80, more preferably from 1 to 50, even more preferably from 1 to 30, and particularly preferably from 1 to 10, and the same applies to a case in which the amino acids are added to the N-terminal side and/or the C-terminal side. This also applies to GpUroA1.

The substitution is preferably a conservative substitution. A conservative substitution is a mutual substitution between Phe, Trp, and Tyr when the substitution site is an aromatic amino acid, between Leu, Ile, and Val when the substitution site is a hydrophobic amino acid, between Gln and Asn when the substitution site is a polar amino acid, between Lys, Arg, and His when the substitution site is a basic amino acid, between Asp and Glu when the substitution site is an acidic amino acid, or between Ser and Thr when the substitution site is an amino acid having a hydroxyl group. Examples of the conservative substitution specifically include a substitution of Ala with Ser or Thr, a substitution of Arg with Gln, His, or Lys, a substitution of Asn with Glu, Gln, Lys, His, or Asp, a substitution of Asp with Asn, Glu, or Gln, a substitution of Cys with Ser or Ala, a substitution of Gln with Asn, Glu, Lys, His, Asp, or Arg, a substitution of Glu with Gly, Asn, Gln, Lys, or Asp, a substitution of Gly with Pro, a substitution of His with Asn, Lys, Gln, Arg, or Tyr, a substitution of Ile with Leu, Met, Val, or Phe, a substitution of Leu with Ile, Met, Val, or Phe, a substitution of Lys with Asn, Glu, Gln, His, or Arg, a substitution of Met with Ile, Leu, Val, or Phe, a substitution of Phe with Trp, Tyr, Met, Ile, or Leu, a substitution of Ser with Thr or Ala, a substitution of Thr with Ser or Ala, a substitution of Trp with Phe or Tyr, a substitution of Tyr with His, Phe, or Trp, and a substitution of Val with Met, Ile, or Leu. This also applies to GpUroA1.

Also, as long as GuUroA1 exhibits, as one sub-unit itself, or when constituting a unit along with GuUroA2 and/or GuUroA3, which are other sub-units, activity to dehydroxylate the hydroxyl group at the 4-position of urolithins, the GuUroA1 may be a protein formed from amino acids, the protein having homology of 80% or greater, preferably 90% or greater, more preferably 95% or greater, even more preferably 97% or greater, and particularly preferably 99% or greater, with respect to the overall length of the amino acid sequence represented by SEQ ID NO: 1. This also applies to GpUroA1.

Moreover, as long as GuUroA1 exhibits, as one sub-unit itself, or when constituting a unit along with GuUroA2 and/or GuUroA3, which are other sub-units, activity to dehydroxylate the hydroxyl group at the 4-position of urolithins, the GuUroA1 may be a protein encoded by a polynucleotide that hybridizes with the base sequence represented by SEQ ID NO: 7 under stringent conditions. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize. This also applies to GpUroA1.

The substitutions of the amino acids, the protein homology, and the stringent conditions and the like also apply to GuUroA2. However, with regard to GuUroA2, the “one to a plurality” is preferably from 1 to 12, more preferably from 1 to 11, even more preferably from 1 to 10, yet even more preferably from 1 to 5, and particularly preferably from 1 to 3. This also applies to GpUroA2.

In addition, the substitutions of the amino acids, the protein homology, and the stringent conditions and the like also apply to GuUroA3. However, with regard to GuUroA3, the “one to a plurality” is preferably from 1 to 7, more preferably from 1 to 6, even more preferably from 1 to 5, yet even more preferably from 1 to 3, and particularly preferably from 1 to 2. This also applies to GpUroA3.

The activity of dehydroxylating the hydroxyl group at the 4-position of urolithins through the enzyme according to the present aspect can be evaluated, for example, as in Experimental Example 5 described below.

### <1-1-2 (A). Polynucleotide>

Another aspect of the present disclosure is a polynucleotide containing:
one or more base sequences selected from the group consisting of a base sequence represented by SEQ ID NO: 7, a base sequence represented by SEQ ID NO: 8, and a base sequence represented by SEQ ID NO: 9; or
one or more base sequences selected from a base sequence represented by SEQ ID NO: 19, a base sequence represented by SEQ ID NO: 20, and a base sequence represented by SEQ ID NO: 21.

The details of each base sequence are as described in the previous section “1-1-1 (A). Enzyme that dehydroxylates the hydroxyl group at the 4-position of urolithins”.

As long as GuUroA1 exhibits, as one sub-unit itself, or when constituting a unit along with GuUroA2 and/or GuUroA3, which are other sub-units, activity to dehydroxylate the hydroxyl group at the 4-position of urolithins, the base sequence of the gene encoding GuUroA1 may be a base sequence that hybridizes with the base sequence represented by SEQ ID NO: 7 under stringent conditions. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize.

This applies also to the base sequence of the gene encoding GuUroA2 and the base sequence of the gene encoding GuUroA3. In addition, this also applies with regard to the base sequence of the gene encoding GpUroA1, the base sequence of the gene encoding GpUroA2, and the base sequence of the gene encoding GpUroA3.

### <1-1-3 (A). Genetic Engineering Aspects>

Other aspects of the present disclosure include a recombinant vector containing the polynucleotide, a transformant in which the polynucleotide is expressively retained, or in which the vector is expressively retained, and a method for producing a protein encoded by the polynucleotide, the method including a step of culturing the transformant. The protein is the “enzyme that dehydroxylates the hydroxyl group at the 4-position of urolithins”.

An expression vector that expresses the enzyme can be formed by inserting the polynucleotide into a known expression vector. In addition, the transformant can be obtained by transforming a microorganism or the like using the expression vector, and the transformant can then be cultured, etc. to produce the enzyme, and thereby the enzyme can be obtained.

Examples of host vector systems include, as lactic acid bacteria, Lactococcus lactis subsp. Cremoris-vector-pNZ8148 (available from MoBiTech, Inc.), Lactococcus lactis-pGKV11 (Appl. Environ. Microbiol., 50, 540-542 (1985)), and Lactococcus lactis/Streptococcus thermophilus/Streptococcus faecalis, and the like—pBE194 (JP 06-253861 A), and Lactococcus lactis subsp. lactis/Lactobacillus delbrueckii, and the like—pSYE1 (JP 05-176776 A), and as bifidobacteria, Bifidobacterium adolescentis-pKKT427 (Nucleic Acids Research, 2009, Vol. 37, No. 1 e3 doi: 10.1093/nar/gkn884), a wide-range of Lactobacillus bifidus-pNC7 such as Bifidobacterium longum (Res. Microbiol., 147, 133-143 (1996)), and Bifidobacterium longum-pBS423 (Appl. Environ. Microbiol., 78, 4984-4994 (2012)).

Examples of anaerobic bacteria include Clostridium acetobutylicum—pTY10 (Agric. Biol. Chem., 54 (2), 437-441 (1990))/pMTL500E (Microbiol. SCI. 5: 310-315 (1988)), and ACE vector (Anaerobe, 41, 104-112 (2016)).

While expression of the enzyme requires an anaerobic environment, heterologous expression of the gene does not necessarily require an anaerobic host-vector system. For example, the Rhodococcus erythropolis—pTip, pNit, pCpi Vector Series (Hokkaido System Science Co., Ltd., Biotechnol. Bioeng., 86, 136-148 (2004)) can be suitably used. A specific example of the host includes the L88 strain, and a specific example of the vector includes pTipQC1.

In addition to microorganisms, various hosts and vector systems have been developed with respect to plants and animals. For example, a system in which silkworms are used (Nature 315, 592-594 (1985)), and a system that expresses heterologous proteins in large amounts in plants such as rapeseed, corn, and potatoes have been developed, and these system may be used.

### <1-2-1 (A). Method for Dehydroxylating the Hydroxyl Group at the 4-Position of Urolithins>

Another aspect of the present disclosure is a method for dehydroxylating the hydroxyl group at the 4-position of urolithins. More specifically, the method is a method in which the hydroxyl group at the 4-position of urolithins having a hydroxyl group at the 4-position is dehydroxylated.

The present aspect is a method for dehydroxylating a hydroxyl group at the 4-position of urolithins, the method including the following Step (I):
Step (I): bringing one or more components selected from the following (i) to (iv) into contact with urolithins having a hydroxyl group at the 4-position, so as to dehydroxylate the hydroxyl group at the 4-position:
(i) the enzyme described in the section “1-1-1 (A). Enzyme that dehydroxylates the hydroxyl group at the 4-position of urolithins”;
(ii) a protein encoded by a polynucleotide described in the section “1-1-2 (A). Polynucleotide”;
(iii) a microorganism that produces the enzyme described in (i) above or the protein described in (ii) above; and
(iv) a treated product of the microorganism described in (iii) above.

### (Urolithins)

The details of the urolithins in the present aspect are as described in the previously mentioned “1-1-1 (A). Enzyme that dehydroxylates the hydroxyl group at the 4-position of urolithins”.

### (Step (I))

In Step (I), (i) the enzyme, and (ii) the protein encoded by the polynucleotide contact each of the urolithins having a hydroxyl group at the 4-position and dehydroxylate the hydroxyl group present in the urolithins at the 4-position. In addition, with (iii) the microorganism that produces the enzyme described in (i) above or the protein described in (ii) above and (iv) the treated product of the microorganism described in (iii) above, the enzyme or protein contained in the microorganism or in the treated product of the microorganism contacts the urolithins having a hydroxyl group at the 4-position, and thereby dehydroxylates the hydroxyl group present in the urolithins at the 4-position.

With respect to (iii) the microorganism that produces the enzyme described in (i) above or the protein described in (ii) above and (iv) the treated product of the microorganism described in (iii) above, the microorganism may be obtained through a genetic engineering technique.

Examples of such a microorganism include a microorganism belonging to the genus Gordonibacter. Specific preferable examples of the microorganism belonging to the genus Gordonibacter include the same microorganism as those described in the “1-1-1 (A). Enzyme that dehydroxylates the hydroxyl group at the 4-position of urolithins” section above.

The (i) enzyme and the (ii) protein encoded by a polynucleotide are not limited those that have been purified, and may also include those that have been partially purified.

Furthermore, examples of (iv) the treated product of the microorganism include the microorganism in which the permeability of the cell membrane has been changed by treatment using a surfactant or an organic solvent such as toluene, a cell-free extract in which bacterial cells have been crushed by a treatment using glass beads or enzymes, or a treated product obtained by partially purifying the same.

For a case in which one or more components selected from the aforementioned (i) to (iv) are to be brought into contact with urolithins having a hydroxyl group at the 4-position, such contact can be implemented in water; in an organic solvent that does not easily dissolve in water, for example, in an organic solvent such as ethyl acetate, butyl acetate, toluene, chloroform, and n-hexane; or in a two-phase mixed system of the organic solvent and an aqueous medium such as ethanol or acetone. The contact can also be implemented by immobilizing the one or more components selected from the aforementioned (i) to (iv), or the contact can be implemented using a membrane reactor or the like.

Furthermore, if the one or more components selected from (i) to (iv) are the microorganism that produces the enzyme described in (i) above or the protein described in (ii) above, the contact can be implemented in a culture medium or a solution normally used in culturing the microorganism, such as a phosphate buffer.

The temperature in Step (I) is preferably 25 °C or higher, more preferably 30 °C or higher, and even more preferably 33 °C or higher, and is also preferably 55 °C or lower, more preferably 50 °C or lower, and even more preferably 42 °C or lower.

The pH in Step (I) is preferably 4.0 or higher, more preferably 5.0 or higher, and even more preferably 5.5 or higher, and is also preferably 9.0 or less, more preferably 8.0 or less, and even more preferably 7.0 or less.

In Step (I), the concentration in the reaction solution of urolithins having a hydroxyl group at the 4-position is 0.001 g/L or greater, preferably 0.01 g/L or greater, and more preferably 0.1 g/L or greater, and is also not greater than 100 g/L, preferably not greater than 20 g/L, and more preferably not greater than 10 g/L.

The present aspect may include a step of quantitatively determining the resulting product (quantitative determination step). The quantification method can be in accordance with routine methods. For example, ethyl acetate to which an acid such as formic acid has been added as necessary, is added to the culturing solution and stirred well, after which the mixture is centrifuged, and the ethyl acetate layer is extracted. The same operation can be implemented several times as necessary, and the extracted ethyl acetate layers can be combined to obtain a liquid extract of the product. The liquid extract is concentrated under reduced pressure using an evaporator or the like, dried, and dissolved in methanol. As an example, the obtained solution is then filtered using a membrane such as a polytetrafluoroethylene (PTFE) membrane to remove insoluble matter, and the resulting product can then be quantified using high performance liquid chromatography.

The present aspect may also include a step of recovering the resulting product. The recovery step includes steps such as a purification step and a concentration step. As a purification treatment in the purification step, treatments such as sterilization of the microorganism through heat; disinfection through a method such as microfiltration (MF) or ultrafiltration (UF); removal of solids and polymeric substances; extraction using an organic solvent, an ionic liquid, or the like; and adsorption and decolorization using a hydrophobic adsorbent, an ion exchange resin, an activated carbon column, or the like can be implemented. Furthermore, examples of a concentration treatment in the concentration step include concentration using an evaporator, reverse osmosis membrane, or the like.

In addition, a solution containing the resulting product can be formed into a powder through freeze drying, spray drying, or the like. In the formation of a powder, an excipient such as lactose, dextrin, or corn starch can be added.

<1-1-1 (B). Enzyme that Dehydroxylates the Hydroxyl Group at the 10-Position of Urolithins>

Another aspect of the present disclosure is an enzyme that dehydroxylates the hydroxyl groups at the 10-position of urolithins. More specifically, the enzyme is an enzyme that catalyzes a reaction in which the hydroxyl group at the 10-position of urolithins having a hydroxyl group at the 10-position is dehydroxylated. It is speculated that the enzyme thereof is constituted from one sub-unit, two sub-units, or three sub-units.

With regard to the enzyme according to the present aspect, refer to the details described in the “1-1-1 (A). Enzyme that dehydroxylates the hydroxyl group at the 4-position of urolithins” section. However, differences also exist, and thus those differences are described.

The urolithins in the present aspect are preferably urolithin M5, urolithin M6, or urolithin M7. When the hydroxyl group at the 10-position is dehydroxylated by the enzyme according to the present aspect, urolithin D, urolithin C, and urolithin A are respectively produced.

The enzyme according to the present aspect is activated by a cofactor. Examples of the cofactor include methyl viologen (MV), reduced nicotinamide adenine dinucleotide (NADH), reduced nicotinamide adenine dinucleotide phosphate (NADPH), flavin adenine dinucleotide (FAD), and flavin adenine mononucleotide (FMN).

With the cofactor of the present aspect, the activity of dehydroxylation of the hydroxyl group at the 10-position of the urolithins is increased, and therefore MV and NADH are preferably used, MV, NADPH and FMN are more preferably used, MV and NADPH are even more preferably used, MV, NADPH, and FAD are yet even more preferably used, MV, NADH and FMN are particularly preferably used, and MV, NADPH and FAD are even more particularly preferably used.

The enzyme according to the present aspect preferably has the properties (3) and (4) below.

(3) The optimum pH is preferably 5.0 or higher, and more preferably 5.5 or higher, and is preferably 7.0 or less, more preferably 6.5 or less, and even more preferably 6.0 or less.

(4) The optimum temperature is preferably 37 °C or higher, and more preferably 42 °C or higher, and is also preferably 50 °C or lower, and more preferably 42 °C or lower.

The enzyme according to the present aspect preferably has the property of (5) below.

(5) A SDS-PAGE result includes a band indicating a molecular weight of preferably 78,000 or greater, more preferably 82,000 or greater, and even more preferably 84,000 or greater, and also preferably 96,000 or less, more preferably 92,000 or less, and even more preferably 90,000 or less.

The one sub-unit, two sub-units, or three sub-units of the Gordonibacter urolithinfaciens DSM 27213 strain, the sub-units thereof being presumed to constitute the enzyme according to the present aspect, are preferably selected from combinations of GuUroB1, GuUroB2, and GuUroB3.

The amino acid sequences of GuUroB1, GuUroB2, and GuUroB3 are the sequences represented by SEQ ID NOS: 4 to 6, respectively.

The base sequences of the genes encoding GuUroB1, GuUroB2, and GuUroB3 are sequences represented by SEQ ID NOS: 10 to 12, respectively.

Furthermore, as mentioned previously, the enzyme according to the present aspect preferably has the property (4) described in the “1-1-1 (A). Enzyme that dehydroxylates the hydroxyl group at the 4-position of urolithins” section. The molecular weight of 78,000 or greater and 96,000 or less is presumed to be the molecular weight of the GuUroB1 sub-unit.

The one sub-unit, the two sub-units, or the three sub-units of the Gordonibacter pamelaeae DSM 19378 strain, the sub-units thereof being presumed to constitute an enzyme according to the present aspect, are preferably selected from combinations of GpUroB1, GpUroB2 and GpUroB3. The amino acid sequences of GpUroB1, GpUroB2, and GpUroB3 are the sequence represented by SEQ ID NOS: 16 to 18, respectively.

The base sequences of the genes encoding GpUroB1, GpUroB2, and GpUroB3 are the sequences represented by SEQ ID NOS: 22 to 24, respectively.

Accordingly, the enzyme according to the present aspect is preferably an enzyme containing:
one or more amino acid sequence selected from the group consisting of the amino acid sequence represented by SEQ ID NO: 4, the amino acid sequence represented by SEQ ID NO: 5, and the amino acid sequence represented by SEQ ID NO: 6; or
one or more amino acid sequence selected from the group consisting of the amino acid sequence represented by SEQ ID NO: 16, the amino acid sequence represented by SEQ ID NO: 17, and the amino acid sequence represented by SEQ ID NO: 18.

Also, as long as GuUroB1 exhibits, as one sub-unit itself, or when constituting a unit along with GuUroB2 and/or GuUroB3, which are other sub-units, activity to dehydroxylate the hydroxyl group at the 10-position of urolithins, the GuUroB1 may be a protein formed from amino acids, the protein thereof being such that in the amino acid sequence represented by SEQ ID NO: 4, one to a plurality of amino acids are substituted or deleted, or one to a plurality of amino acids are inserted or added. One to a plurality means preferably from 1 to 80, more preferably from 1 to 79, even more preferably from 1 to 50, yet even more preferably from 1 to 30, and particularly preferably from 1 to 10, and the same applies to a case in which the amino acids are added to the N-terminal side and/or the C-terminal side. This also applies to GpUroB1.

Furthermore, the substitutions of amino acids, the homology of proteins, the stringent conditions, and the like are the same as those of the enzyme described in the “1-1-1 (A). Enzyme that dehydroxylates the hydroxyl group at the 4-position of urolithins” section. This also applies to GpUroB1.

In addition, the substitutions of the amino acids, the protein homology, and the stringent conditions and the like also apply to GuUroB2. However, with regard to GuUroB2, the “one to a plurality” is preferably from 1 to 12, more preferably from 1 to 11, even more preferably from 1 to 10, yet even more preferably from 1 to 5, and particularly preferably from 1 to 3. This also applies to GpUroB2.

In addition, the substitutions of the amino acids, the protein homology, and the stringent conditions and the like also apply to GuUroB3. However, with regard to GuUroB3, the “one to a plurality” is preferably from 1 to 20, more preferably from 1 to 19, even more preferably from 1 to 18, yet even more preferably from 1 to 10, particularly preferably from 1 to 5, and even more particularly preferably from 1 to 3. This also applies to GpUroB3.

The activity of dehydroxylating the hydroxyl group at the 10-position of urolithins through the enzyme according to the present aspect can be evaluated, for example, by quantitatively determining the urolithin C produced as a product from urolithin M6, similar to the method described in Experimental Example 5 described below.

### <1-1-2 (B). Polynucleotide>

Another aspect of the present disclosure is a polynucleotide containing:
one or more base sequences selected from the group consisting of a base sequence represented by SEQ ID NO: 10, a base sequence represented by SEQ ID NO: 11, and a base sequence represented by SEQ ID NO: 12; or
one or more base sequences selected from a base sequence represented by SEQ ID NO: 22, a base sequence represented by SEQ ID NO: 23, and a base sequence represented by SEQ ID NO: 24.

The details of each base sequence are as described in the previously mentioned “1-1-1 (B). Enzyme that dehydroxylates the hydroxyl group at the 10-position of urolithins” section.

Moreover, as long as GuUroB1 exhibits, as one sub-unit itself, or when constituting a unit along with GuUroB2 and/or GuUroB3, which are other sub-units, activity to dehydroxylate the hydroxyl group at the 10-position of urolithins, the base sequence of the gene encoding GuUroB1 may be a base sequence that hybridizes with the base sequence represented by SEQ ID NO: 10 under stringent conditions. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize.

This applies also to the base sequence of the gene encoding GuUroB2 and the base sequence of the gene encoding GuUroB3. In addition, this also applies with regard to the base sequence of the gene encoding GpUroB1, the base sequence of the gene encoding GpUroB2, and the base sequence of the gene encoding GpUroB3.

### <1-1-3 (B). Genetic Engineering Aspects>

Other aspects of the present disclosure include a recombinant vector containing the polynucleotide, a transformant in which the polynucleotide is expressively retained, or in which the vector is expressively retained, and a method for producing a protein encoded by the polynucleotide, the method including a step of culturing the transformant. The protein is the “enzyme that dehydroxylates the hydroxyl group at the 10-position of urolithins”.

An expression vector that expresses the enzyme can be formed by inserting the polynucleotide into a known expression vector. The transformant can be obtained by transforming microorganisms or the like using the expression vector, and the transformant can then be cultured, etc. to produce the enzyme, and thereby the enzyme can be obtained.

For other matters, the description of the “1-1-3 (A). Genetic engineering aspects” section is incorporated by reference.

### <1-2-1 (B). Method for Dehydroxylating the Hydroxyl Group at the 10-Position of Urolithins>

Another aspect of the present disclosure is a method for dehydroxylating the hydroxyl group at the 10-position of urolithins. More specifically, the method is a method in which the hydroxyl group at the 10-position of urolithins having a hydroxyl group at the 10-position is dehydroxylated.

The present aspect is a method for dehydroxylating a hydroxyl group at the 10-position of urolithins, the method including the following Step (I):
Step (I): bringing one or more components selected from the following (i) to (iv) into contact with urolithins having a hydroxyl group at the 10-position, so as to dehydroxylate the hydroxyl group at the 10-position:
(i) the enzyme described in the “1-1-1 (B). Enzyme that dehydroxylates the hydroxyl group at the 10-position of urolithins” section;
(ii) a protein encoded by a polynucleotide described in the section “1-1-2 (B). Polynucleotide”;
(iii) a microorganism that produces the enzyme described in (i) above or the protein described in (ii) above; and
(iv) a treated product of the microorganism described in (iii) above.

### (Urolithins)

With regard to the details of the urolithins of the present aspect, the details described in the “1-1-1 (B). Enzyme that dehydroxylates the hydroxyl group at the 10-position of urolithins” section described above are incorporated herein.

### (Step (I))

In Step (I), (i) the enzyme, and (ii) the protein encoded by the polynucleotide contact each of the urolithins having a hydroxyl group at the 10-position, and dehydroxylate the hydroxyl group present in the urolithins at the 10-position. In addition, with (iii) the microorganism that produces the enzyme described in (i) above or the protein described in (ii) above, and (iv) the treated product of the microorganism described in (iii) above, the enzyme or protein contained in the microorganism or in the treated product of the microorganism contacts the urolithins having a hydroxyl group at the 10-position, and thereby dehydroxylates the hydroxyl group present in the urolithins at the 10-position.

With respect to (iii) the microorganism that produces the enzyme described in (i) above or the protein described in (ii) above, and (iv) the treated product of the microorganism described in (iii) above, the microorganism may be obtained through a genetic engineering technique.

Examples of such a microorganism include a microorganism belonging to the genus Gordonibacter, and a microorganism belonging to the genus Eggerthella.

Specific preferable examples of the microorganism belonging to the genus Gordonibacter include the same microorganism as those described in the section “1-1-1 (A). Enzyme that dehydroxylates the hydroxyl group at the 4-position of urolithins” above.

The microorganism belonging to the genus Eggerthella is preferably the Eggerthella sp. DC3563 (NITE BP-02376) strain belonging to the genus Eggerthella but not applicable to an existing species.

Note that an international deposit of the Eggerthella sp. DC3563 (NITE BP-02376) strain was established on the basis of the Budapest Treaty on Nov. 11, 2106 by the National Institute of Technology and Evaluation at the NITE Patent Microorganisms Depositary [Address: #122, 2-5-8 Kazusakamatari, Kisarazu-shi, Chiba 292-0818, Japan].

The Eggerthella sp. DC3563 (NITE BP-02376) strain is not limited to the same strain thereof and may be bacteria that is substantially equivalent to the deposited strain. Substantially equivalent bacteria are microorganisms that belong to the genus Eggerthella, and can exhibit an effect according to the present aspect, such as dehydroxylating the hydroxyl group at the 10-position of urolithins having a hydroxyl group at the 10-position, and furthermore, are microorganisms in which the base sequence of the 16S rRNA gene thereof has homology of preferably 98% or higher, more preferably 99% or higher, and even more 100% in relation to the base sequence of the 16S rRNA gene of the deposited strain, and the microorganisms thereof preferably have the same mycological properties as the deposited strain. Moreover, as long as the effect of the present aspect is not impaired, the strain may be a strain that is bred from the deposited strain or a strain substantially equivalent thereto, by a mutation treatment, genetic recombination, selection of a natural mutant strain, or the like.

For other matters, the description of the section “1-2-1 (A). Method for dehydroxylating the hydroxyl group at the 4-position of urolithins” is incorporated by reference.

### <1-3-1. Urolithin C Production Method (First Aspect)>

Another aspect of the present disclosure is a method of producing urolithin C (first aspect).

The present aspect is a method for producing urolithin C, the method including the following Steps (I) to (III):

Step (I): allowing a microorganism having an ability to produce urolithin M5 from ellagic acid to produce urolithin M5 from ellagic acid.

Step (II): bringing one or more components selected from the following (i) to (iv) into contact with the urolithin M5, so as to produce urolithin M6:
(i) the enzyme described in the section “1-1-1 (A). Enzyme that dehydroxylates the hydroxyl group at the 4-position of urolithins”;
(ii) a protein encoded by a polynucleotide described in the section “1-1-1 (A). Polynucleotide”;
(iii) a microorganism that produces the enzyme described in (i) above or the protein described in (ii) above; and
(iv) a treated product of the microorganism described in (iii) above.

Step (III): bringing one or more components selected from the following (v) to (viii) into contact with the urolithin M6, so as to produce urolithin C:
(v) the enzyme described in the section “1-1-1 (B). Enzyme that dehydroxylates the hydroxyl group at the 10-position of urolithins”;
(vi) a protein encoded by a polynucleotide described in the section “1-1-1 (B). Polynucleotide”;
(vii) a microorganism that produces the enzyme described in (v) above or the protein described in (vi) above; and
(viii) a treated product of the microorganism described in (vii) above.

### (Step (I))

In Step (I), a microorganism having an ability to produce urolithin M5 from ellagic acid is allowed to produce urolithin M5 from ellagic acid.

Examples of the microorganism having an ability to produce urolithin M5 from ellagic acid include the microorganism described in Step (I) in the section entitled “1-2-1 (B). Method for dehydroxylating the hydroxyl group at the 10-position of urolithins”.

As a method for producing urolithin M5 from ellagic acid, a case can be illustrated in which the target for contact with urolithins having a hydroxyl group at the 10-position with regard to the method described in the section “1-2-1 (B). Method for dehydroxylating the hydroxyl group at the 10-position of urolithins” is a microorganism that produces the enzyme described in the section “1-1-1 (B). Enzyme that dehydroxylates the hydroxyl group at the 10-position of urolithins” described above, or a microorganism that produces a protein encoded by the polynucleotide described in the section “1-1-1 (B). Polynucleotide” above.

### (Step (II))

In Step (II), one or more components selected from the following (i) to (iv) are brought into contact with urolithin M5, so as to produce urolithin M6:
(i) the enzyme described in the section “1-1-1 (A). Enzyme that dehydroxylates the hydroxyl group at the 4-position of urolithins”;
(ii) a protein encoded by a polynucleotide described in the section “1-1-1 (A). Polynucleotide”;
(iii) a microorganism that produces the enzyme described in (i) above or the protein described in (ii) above; and
(iv) a treated product of the microorganism described in (iii) above.

In Step (II), (i) the enzyme, and (ii) the protein encoded by the polynucleotide contact each urolithin M5 having a hydroxyl group at the 4-position, and dehydroxylate the hydroxyl group present in the urolithin M5 at the 4-position. In addition, with (iii) the microorganism that produces the enzyme described in (i) above or the protein described in (ii) above, and (iv) the treated product of the microorganism described in (iii) above, the enzyme or protein contained in the microorganism or in the treated product of the microorganism contacts the urolithin M5 having a hydroxyl group at the 4-position, and thereby dehydroxylates the hydroxyl group present in the urolithin M5 at the 4-position.

The details of the aspect thereof are the same as the details described in the section “1-2-1 (A). Method for dehydroxylating the hydroxyl group at the 4-position of urolithins”.

### (Step (III))

In Step (III), one or more components selected from the following (v) to (viii) are brought into contact with the urolithin M6, so as to produce urolithin C:
(v) the enzyme described in the section “1-1-1 (B). Enzyme that dehydroxylates the hydroxyl group at the 10-position of urolithins”;
(vi) a protein encoded by a polynucleotide described in the section “1-1-1 (B). Polynucleotide”;
(vii) a microorganism that produces the enzyme described in (v) above or the protein described in (vi) above; and
(viii) a treated product of the microorganism described in (vii) above.

In Step (III), (v) the enzyme and (vi) the protein encoded by the polynucleotide contact each urolithin M6 having a hydroxyl group at the 10-position, and dehydroxylate the hydroxyl group present in the urolithin M6 at the 10-position. In addition, with (vii) the microorganism that produces the enzyme described in (v) above or the protein described in (vi) above and (viii) the treated product of the microorganism described in (vii) above, the enzyme or protein contained in the microorganism or in the treated product of the microorganism contacts the urolithin M6 having a hydroxyl group at the 10-position, and thereby dehydroxylates the hydroxyl group present in the urolithin M6 at the 10-position.

The details of the aspect thereof are the same as the details described in the section “1-2-1 (B). Method for dehydroxylating the hydroxyl group at the 10-position of urolithins”.

One or more of the steps selected from the group consisting of Step (I) through Step (III) may be implemented in the same system.

The above “implemented in the same system” means that with regard to the series in which the urolithin M5 produced in Step (I) is used as is as the urolithin M5 in Step (II) to produce the urolithin M6 in Step (II), and the urolithin M6 is then used as is as the urolithin M6 in Step (III) to produce urolithin C in Step (III), the series thereof is continuously implemented in the same system. That is, for example, this series does not include a step of separating and/or purifying the urolithin M5 produced in Step (I). Note that when microorganisms are used in each step, the microorganisms in each step may be the same or different.

The present aspect may include other steps. For example, a step of quantitatively determining the obtained urolithin C (quantitative determination step), a step of purifying the obtained urolithin C (purification step), and a step of concentrating the urolithin C (concentration step) may be included. The details of these steps are the same as the details described in the section “1-2-1 (A). Method for dehydroxylating the hydroxyl group at the 4-position of urolithins” above. Further, a step of forming a solution containing urolithin C into a powder through freeze drying, spray drying, or the like may also be included. In the formation of a powder, an excipient such as lactose, dextrin, or corn starch can be added.

### <1-3-2. Urolithin C Production Method (Second Aspect)>

Another aspect of the present disclosure is a method of producing C (second aspect).

The present aspect is a method for producing urolithin C, the method including the following Steps (I) to (III).

Step (I): allowing a microorganism having an ability to produce urolithin M5 from ellagic acid to produce urolithin M5 from ellagic acid.

Step (II): bringing one or more components selected from the following (i) to (iv) into contact with the urolithin M5, so as to produce urolithin D:
(i) the enzyme described in the section “1-1-1 (B). Enzyme that dehydroxylates the hydroxyl group at the 10-position of urolithins”;
(ii) a protein encoded by a polynucleotide described in the section “1-1-1 (B). Polynucleotide”;
(iii) a microorganism that produces the enzyme described in (i) above or the protein described in (ii) above; and
(iv) a treated product of the microorganism described in (iii) above.

Step (III): bringing one or more components selected from the following (v) to (viii) into contact with the urolithin D, so as to produce urolithin C:
(v) the enzyme described in the section “1-1-1 (A). Enzyme that dehydroxylates the hydroxyl group at the 4-position of urolithins”;
(vi) a protein encoded by a polynucleotide described in the section “1-1-1 (A). Polynucleotide”;
(vii) a microorganism that produces the enzyme described in (i) above or the protein described in (v) above; and
(viii) a treated product of the microorganism described in (vii) above.

### (Step (I))

Regarding Step (I), the details described in the aforementioned “1-3-1. Urolithin C production method (first aspect)” are incorporated by reference.

### (Step (II))

In Step (II), one or more components selected from the following (i) to (iv) are contacted with urolithin M5 to produce urolithin D:
(i) the enzyme described in the section “1-1-1 (B). Enzyme that dehydroxylates the hydroxyl group at the 10-position of urolithins”;
(ii) a protein encoded by a polynucleotide described in the section “1-1-1 (B). Polynucleotide”;
(iii) a microorganism that produces the enzyme described in (i) above or the protein described in (ii) above; and
(iv) a treated product of the microorganism described in (iii) above.

In Step (II), (i) the enzyme and (ii) the protein encoded by the polynucleotide contact each urolithin M5 having a hydroxyl group at the 10-position, and dehydroxylate the hydroxyl group present in the urolithin M5 at the 10-position. In addition, with (iii) the microorganism that produces the enzyme described in (i) above or the protein described in (ii) above and (iv) the treated product of the microorganism described in (iii) above, the enzyme or protein contained in the microorganism or in the treated product of the microorganism contacts the urolithin M5 having a hydroxyl group at the 10-position, and thereby dehydroxylates the hydroxyl group present in the urolithin M5 at the 10-position.

The details of the aspect thereof are the same as the details described in the section “1-2-1 (B). Method for dehydroxylating the hydroxyl group at the 10-position of urolithins”.

In Step (III), one or more components selected from the following (v) to (viii) are contacted with the urolithin D to produce C:
(v) the enzyme described in the section “1-1-1 (A). Enzyme that dehydroxylates the hydroxyl group at the 4-position of urolithins”;
(vi) a protein encoded by a polynucleotide described in the section “1-1-1 (A). Polynucleotide”;
(vii) a microorganism that produces the enzyme described in (i) above or the protein described in (v) above; and
(viii) a treated product of the microorganism described in (vii) above.

In Step (III), (v) the enzyme and (vi) the protein encoded by the polynucleotide contact each urolithin D having a hydroxyl group at the 4-position, and dehydroxylate the hydroxyl group present in the urolithin D at the 4-position. In addition, with (vii) the microorganism that produces the enzyme described in (vi) above or the protein described in (vii) above and (viii) the treated product of the microorganism described in (vii) above, the enzyme or protein contained in the microorganism or in the treated product of the microorganism contacts the urolithin D having a hydroxyl group at the 4-position, and thereby dehydroxylates the hydroxyl group present in the urolithin D at the 4-position.

The details of the aspect thereof are the same as the details described in the section “1-2-1 (A). Method for dehydroxylating the hydroxyl group at the 4-position of urolithins”.

The Steps (I) to (III) may be implemented in the same system.

The above “implemented in the same system” means that with regard to the series in which the urolithin M5 produced in Step (I) is used as is as the urolithin M5 in Step (II) to produce the urolithin D in Step (II), and the urolithin D is then used as is as the urolithin D in Step (III) to produce urolithin C in Step (III), the series thereof is continuously implemented in the same system. That is, for example, this series does not include a step of separating and/or purifying the urolithin M5 produced in Step (I). Note that when microorganisms are used in each step, the microorganisms in each step may be the same or different.

The present aspect may include other steps. The details of these steps are the same as the details described in the section “1-3-1. Urolithin C production method (first aspect)”.

### <1-3-3. Urolithin A Production Method (First Aspect)>

Another aspect of the present disclosure is a method (first aspect) for producing urolithin A.

The present aspect is a method for producing urolithin A, the method including Step (I).

Step (I): allowing a microorganism having an ability to produce urolithin A from urolithin C to produce urolithin A from the urolithin C produced by the method described in “1-3-1. Urolithin C production method (first aspect)”.

The steps of the “1-3-1. Urolithin C production method (first aspect)” of the present aspect are described below. In this case, Step (I) described above corresponds to Step (IV) below.

Namely, the present aspect is a method for producing urolithin A, the method including Steps (I) to (IV):

Step (I): allowing a microorganism having an ability to produce urolithin M5 from ellagic acid to produce urolithin M5 from ellagic acid.

Step (II): bringing one or more components selected from the following (i) to (iv) into contact with the urolithin M5, so as to produce urolithin M6:
(i) the enzyme described in the section “1-1-1 (A). Enzyme that dehydroxylates the hydroxyl group at the 4-position of urolithins”; (ii) a protein encoded by a polynucleotide described in the section “1-1-1 (A). Polynucleotide”;
(iii) a microorganism that produces the enzyme described in (i) above or the protein described in (ii) above; and
(iv) a treated product of the microorganism described in (iii) above.

Step (III): bringing one or more components selected from the following (v) to (viii) into contact with the urolithin M6, so as to produce urolithin C:
(v) the enzyme described in the section “1-1-1 (B). Enzyme that dehydroxylates the hydroxyl group at the 10-position of urolithins”; (vi) a protein encoded by a polynucleotide described in the section “1-1-1 (B). Polynucleotide”;
(vii) a microorganism that produces the enzyme described in (v) above or the protein described in (vi) above; and
(viii) a treated product of the microorganism described in (vii) above.

Step (IV): allowing a microorganism having an ability to produce urolithin A from urolithin C to produce urolithin A from the urolithin C.

### (Step (I) to Step (III))

Regarding Step (I) to Step (III), the details described in the aforementioned “1-3-1. Urolithin C production method (first aspect)” are incorporated by reference.

### (Step (IV))

In Step (IV), a microorganism having an ability to produce urolithin A from urolithin C is allowed to produce urolithin A from urolithin C.

Examples of the microorganism having an ability to produce urolithin A from urolithin C include a microorganism belonging to the genus Clostridium.

Even among the microorganism belonging to the genus Clostridium, a microorganism belonging to Clostridium bolteae, a microorganism belonging to Clostridium asparagiforme, and a microorganism belonging to Clostridium citroniae are preferable.

Furthermore, with regard to the microorganism belonging to Clostridium bolteae, a DSM 29485 strain, a DSM 15670 strain, and a JCM 12243 strain are more preferable, and the DSM 15670 strain is even more preferable.

Among the microorganism belonging to Clostridium asparagiforme, a DSM 15981 strain is preferable.

Among the microorganism belonging to Clostridium citroniae, a DSM 19261 strain is preferable.

As a microorganism of Clostridium sp. belonging to the genus Clostridium but not applicable to an existing species, the Clostridium sp. DC 3656 (NITE ABP-02708) strain is preferable.

A deposit of the Clostridium sp. DC 3656 strain (NITE ABP-02708) was established in Japan with the depository number NITE P-02708 on 8 May 2018 by the National Institute of Technology and Evaluation (NITE) at the NITE Patent Microorganisms Depositary [Address: #122, 2-5-8 Kazusakamatari, Kisarazu-shi, Chiba 292-0818, Japan]. Subsequently, a request (date received: 15 Jul. 2020) was made for a transfer of control of the same strain to an international depository on the basis of the Budapest Treaty by the National Institute of Technology and Evaluation of the NITE Patent Microorganisms Depositary [Address: #122, 2-5-8 Kazusakamatari, Kisarazu-shi, Chiba 292-0818, Japan], and a receipt number of NITE ABP-02708 was assigned to the strain.

Clostridium sp. DC 3656 (NITE ABP-02708) is not limited to the same strain thereof and may be bacteria that substantially equivalent to the deposited strain. Substantially equivalent bacteria are microorganisms that belong to the genus Clostridium and can exhibit an effect according to the present aspect, such as being able to produce urolithin A from urolithin C, and furthermore, are microorganisms in which the base sequence of the 16S rRNA gene thereof has homology of preferably 98% or higher, more preferably 99% or higher, and even more 100% in relation to the base sequence of the 16S rRNA gene of the deposited strain, and the microorganisms thereof preferably have the same mycological properties as the deposited strain. Moreover, as long as the effect of the present disclosure is not impaired, the strain may be a strain that is bred from the deposited strain or a strain substantially equivalent thereto, by a mutation treatment, genetic recombination, selection of a natural mutant strain, or the like.

This also applies to the Clostridium bolteae DSM 29485 strain and DSM 15670 strain, the JCM 12243 strain, the Clostridium asparagiforme DSM 15981 strain, and the Clostridium citroniae DSM19261 strain.

As a method for producing urolithin A from urolithin C, a case can be illustrated in which the target for contact with urolithins having a hydroxyl group at the 4-position with regard to the method described in section “1-2-1 (A). Method for dehydroxylating the hydroxyl group at the 4-position of urolithins” is a microorganism that produces the enzyme described in the section “1-1-1 (A). Enzyme that dehydroxylates the hydroxyl group at the 4-position of urolithins” described above, or a microorganism that produces a protein encoded by the polynucleotide described in the section “1-1-1 (A). Polynucleotide” above.

One or more of the steps selected from the group consisting of Step (I) through Step (IV) may be implemented in the same system.

The above “implemented in the same system” means that with regard to the series in which the urolithin M5 produced in Step (I) is used as is as the urolithin M5 in Step (II) to produce urolithin M6, the urolithin M6 produced in Step (II) is then used as is as the urolithin M6 in Step (III) to produce urolithin C, and the urolithin C produced in Step (III) is then used as is as the urolithin C in Step (IV) to produce urolithin A in Step (IV), the series thereof is continuously implemented in the same system. That is, for example, this series does not include a step of separating and/or purifying the urolithin M5 produced in Step (I). Note that when microorganisms are used in each step, the microorganisms in each step may be the same or different.

The present aspect may include other steps. The details of these steps are the same as the details described in the section “1-3-1. Urolithin C production method (first aspect)”.

### <1-3-4. Urolithin A Production Method (Second Aspect)>

Another aspect of the present disclosure is a method (second aspect) for producing urolithin A.

The present aspect is a method for producing urolithin A, the method including Step (I):

Step (I): allowing a microorganism having an ability to produce urolithin A from urolithin C to produce urolithin A from the urolithin C produced by the method described in “1-3-2. Urolithin C production method (second aspect)”.

The steps of the “1-3-2. Urolithin C production method (second aspect)” of the present aspect are described below. In this case, Step (I) described above corresponds to Step (IV) below.

Namely, the present aspect is a method for producing urolithin A, the method including Steps (I) to (IV):

Step (I): allowing a microorganism having an ability to produce urolithin M5 from ellagic acid to produce urolithin M5 from ellagic acid.

Step (II): bringing one or more components selected from the following (i) to (iv) into contact with the urolithin M5, so as to produce urolithin D:
(i) the enzyme described in the section “1-1-1 (B). Enzyme that dehydroxylates the hydroxyl group at the 10-position of urolithins”;
(ii) a protein encoded by a polynucleotide described in the section “1-1-1 (B). Polynucleotide”;
(iii) a microorganism that produces the enzyme described in (i) above or the protein described in (ii) above; and
(iv) a treated product of the microorganism described in (iii) above.

Step (III): bringing one or more components selected from the following (v) to (viii) into contact with the urolithin D, so as to produce urolithin C:
(v) the enzyme described in the section “1-1-1 (A). Enzyme that dehydroxylates the hydroxyl group at the 4-position of urolithins”;
(vi) a protein encoded by a polynucleotide described in the section “1-1-1 (A). Polynucleotide”;
(vii) a microorganism that produces the enzyme described in (i) above or the protein described in (v) above; and
(viii) a treated product of the microorganism described in (vii) above.

Step (IV): allowing a microorganism having an ability to produce urolithin A from urolithin C to produce urolithin A from the urolithin C.

### (Step (I) to Step (III))

Regarding Step (I) to Step (III), the details described in the aforementioned “1-3-1. Urolithin C production method (first aspect)” are incorporated by reference.

### (Step (IV))

In Step (IV), a microorganism having an ability to produce urolithin A from urolithin C is allowed to produce urolithin A from urolithin C. Regarding Step (IV), the details described in the aforementioned “1-3-3. Urolithin A production method (first aspect)” section are incorporated by reference.

One or more of the steps selected from the group consisting of Step (I) through Step (IV) may be implemented in the same system.

The above “implemented in the same system” means that with regard to the series in which the urolithin M5 produced in Step (I) is used as is as the urolithin M5 in Step (II) to produce urolithin D, the urolithin D produced in Step (II) is then used as is as the urolithin D in Step (III) to produce urolithin C, and the urolithin C produced in Step (III) is then used as is as the urolithin C in Step (IV) to produce urolithin A in Step (III), the series thereof is continuously implemented in the same system. That is, for example, this series does not include a step of separating and/or purifying the urolithin M5 produced in Step (I). Note that when microorganisms are used in each step, the microorganisms in each step may be the same or different.

The present aspect may include other steps. The details of these steps are the same as the details described in the section “1-3-1. Urolithin C production method (first aspect)”.

### <2-1-1. Ellagic Acid Transporter>

Another aspect of the present disclosure is an ellagic acid transporter.

The ellagic acid transporter according to the present aspect is preferably derived from a microorganism belonging to the genus Gordonibacter.

The ellagic acid transporter thereof is more preferably one or more selected from the group consisting of: a microorganism belonging to Gordonibacter urolithinfaciens; a microorganism belonging to Gordonibacter pamelaeae; and a microorganism belonging to Gordonibacter faecihominis.

The microorganism belonging to Gordonibacter urolithinfaciens is preferably of the Gordonibacter urolithinfaciens DSM 27213 strain.

The microorganism belonging to Gordonibacter pamelaeae is preferably of the DSM 19378 strain.

The microorganism belonging to Gordonibacter faecihominis is preferably of the JCM 16058 strain.

The amino acid sequence of the ellagic acid transporter derived from the Gordonibacter urolithinfaciens DSM 27213 strain is the sequence represented by SEQ ID NO: 25. Also, the base sequence of the gene encoding the ellagic acid transporter thereof is the sequence represented by SEQ ID NO: 26.

The amino acid sequence of the ellagic acid transporter derived from the Gordonibacter pamelaeae DSM 19378 strain is the sequence represented by SEQ ID NO: 27. Also, the base sequence of the gene encoding the ellagic acid transporter thereof is the sequence represented by SEQ ID NO: 28.

Therefore, the ellagic acid transporter according to the present aspect is preferably a protein (an ellagic acid transporter) containing the amino acid sequence represented by SEQ ID NO: 25 or containing the amino acid sequence represented by SEQ ID NO: 27.

Also, the ellagic acid transporter derived from the Gordonibacter urolithinfaciens DSM 27213 strain may be a protein formed from amino acids, the protein thereof being such that in the amino acid sequence represented by SEQ ID NO: 25, one to a plurality of amino acids are substituted or deleted, or one to a plurality of amino acids are inserted or added, as long as the protein exhibits an ellagic acid transporter activity. One to a plurality means preferably from 1 to 50, more preferably from 1 to 49, even more preferably from 1 to 48, yet even more preferably from 1 to 30, particularly preferably from 1 to 10, and more particularly preferably from 1 to 5, and the same applies to a case in which the amino acids are added to the N-terminal side and/or the C-terminal side.

The details of an aspect of substitution are the same as the details described in the section “1-1-1 (A). Enzyme that dehydroxylates the hydroxyl group at the 4-position of urolithins” above.

Also, the ellagic acid transporter derived from the Gordonibacter urolithinfaciens DSM 27213 strain may be a protein having homology of 80% or greater, preferably 90% or greater, more preferably 95% or greater, even more preferably 97% or greater, and particularly preferably 99% or greater in relation to the entire length of the amino acid sequence represented by SEQ ID NO: 25, as long as the protein exhibits an ellagic acid transporter activity.

Also, the ellagic acid transporter derived from the Gordonibacter urolithinfaciens DSM 27213 strain may be a protein encoded by a polynucleotide that hybridizes under stringent conditions with a base sequence represented by SEQ ID NO: 26, as long as the protein exhibits an ellagic acid transporter activity. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize.

The details regarding substitutions of the amino acids, protein homology, stringent conditions, and the like are also applicable to an ellagic acid transporter derived from the Gordonibacter pamelaeae DSM 19378 strain. However, with regard to an ellagic acid transporter derived from the DSM 19378 strain, the “one to a plurality” is preferably from 1 to 52, more preferably from 1 to 51, even more preferably from 1 to 50, yet even more preferably from 1 to 30, particularly preferably from 1 to 10, and even more particularly preferably from 1 to 5.

The ellagic acid transporter activity from an ellagic acid transporter according to the present aspect can be evaluated, for example, as described in Experimental Example 18-1 below.

### <2-1-2. Polynucleotide Containing a Gene Encoding an Ellagic Acid Transporter>

Another aspect of the present disclosure is a polynucleotide containing a base sequence represented by SEQ ID NO: 26 or containing a base sequence represented by SEQ ID NO: 28. The details of each base sequence are as previously described.

The base sequence of a gene encoding the ellagic acid transporter derived from the Gordonibacter urolithinfaciens DSM 27213 strain may be a base sequence that hybridizes with the base sequence represented by SEQ ID NO: 26 under stringent conditions, as long as the protein encoded by the gene thereof exhibits an ellagic acid transporter activity. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize.

This matter is also applicable to a base sequence of a gene encoding an ellagic acid transporter derived from the Gordonibacter pamelaeae DSM 19378 strain.

### <2-1-3. Genetic Engineering Aspects>

Other aspects of the present disclosure include a recombinant vector containing the polynucleotide described in the section “2-1-2. Polynucleotide containing a gene encoding an ellagic acid transporter”, a transformant in which the polynucleotide described in the section “2-1-2. Polynucleotide containing a gene encoding an ellagic acid transporter” is expressively retained, or in which the vector is expressively retained, and a method for producing a protein encoded by the polynucleotide, the method including a step of culturing the transformant. The protein is the “ellagic acid transporter”.

The details of these aspects are similar to the details described in the “1-1-3 (A). Genetic engineering aspects” section.

The transformant promotes the uptake of ellagic acid into the cell thereof from outside the cell. Accordingly, another preferable aspect is a method of promoting the uptake of ellagic acid into the cell of the transformant from outside the cell, the method including the following Step (I) below:

Step (I): bringing the transformant into contact with ellagic acid.

The details of the transformant are similar to the details described in the previously mentioned “1-1-3 (A). Genetic engineering aspects” section. Furthermore, an example of an “aspect in which the transformant is caused into contact with ellagic acid” is an aspect in which the microorganism that produces the enzyme described in (i) or the microorganism that produces the protein described in (ii) are used in the method described in the section “1-2-1 (A). Method for dehydroxylating the hydroxyl group at the 4-position of urolithins”.

### <2-1-4. Urolithin M5 Production Method (First Aspect)>

Another aspect of the present disclosure is a method (first aspect) for producing urolithin M5.

The present aspect is a method for producing urolithin M5, the method including the following Step (I):

Step (I): bringing the transformant described in the section “2-1-3. Genetic engineering aspects”, wherein host of the transformant is a microorganism having an ability to produce urolithin M5 from ellagic acid, into contact with ellagic acid, so as to produce urolithin M5 from the ellagic acid.

### (Step (I))

In Step (I), a transformant described in the section “2-1-3. Genetic engineering aspects”, wherein host of the transformant is a microorganism having an ability to produce urolithin M5 from ellagic acid, is brought into contact with ellagic acid, so as to produce urolithin M5 from the ellagic acid.

The microorganism, which is the host and has the ability to produce urolithin M5 from ellagic acid, may be E. coli, which is commonly used as a host for transformation, but other examples can include the microorganism described in the section “1-2-1 (B). Method for dehydroxylating the hydroxyl group at the 10-position of urolithins” above.

Furthermore, an example of an aspect of contact between the transformant and ellagic acid is an aspect in which the microorganism that produces the enzyme described in (i) or the microorganism that produces the protein described in (ii) are used in the method described in the section “1-2-1 (A). Method for dehydroxylating the hydroxyl group at the 4-position of urolithins”.

The present aspect may include other steps. The details of these steps are the same as the details described in the section “1-3-1. Urolithin C production method (first aspect)”. Also, for example, urolithins may be produced in accordance with the method for producing urolithins described in the present specification, using the urolithin M5 that is produced. In addition, the present aspect may be implemented with a step of producing urolithin M5 from an ellagic acid in the method for producing urolithins described herein.

### <2-1-5. Urolithin C Production Method (Third Aspect)>

Another aspect of the present disclosure is a method for producing urolithin C (third aspect).

The present aspect is a method for producing urolithin C, the method including the following Step (I):

Step (I): bringing the transformant described in the section “2-1-3. Genetic engineering aspects”, wherein host of the transformant is a microorganism having an ability to produce urolithin C from ellagic acid, into contact with ellagic acid, so as to produce urolithin C from the ellagic acid.

### (Step (I))

In Step (I), a transformant described in the section “2-1-3. Genetic engineering aspects”, wherein host of the transformant is a microorganism having an ability to produce urolithin C from ellagic acid, is brought into contact with ellagic acid.

The microorganisms having the ability to produce urolithin C from ellagic acid may be E. coli, which is commonly used as a host for transformation, but other examples can include the microorganisms described in the section “1-3-1. Urolithin C production method (first aspect)” above.

Furthermore, an example of an aspect of contact between the transformant and ellagic acid is an aspect in which the microorganism that produces the enzyme described in (i) or the microorganism that produces the protein described in (ii) are used in the method described in the section “1-2-1 (A). Method for dehydroxylating the hydroxyl group at the 4-position of urolithins”.

The present aspect may include other steps. The details of these steps are the same as the details described in the section “1-3-1. Urolithin C production method (first aspect)”. Also, for example, urolithins may be produced in accordance with the method for producing urolithins described in the present specification, using the urolithin C that is produced. In addition, the present aspect may be implemented with a step of producing urolithin C from an ellagic acid in the method for producing urolithins described herein.

### <2-1-6. Urolithin A Production Method (Third Aspect)>

Another aspect of the present disclosure is a method for producing urolithin A (third aspect).

The present aspect is a method for producing urolithin A, the method including the following Step (I):

Step (I): allowing a microorganism having the ability to produce urolithin A from urolithin C to produce urolithin A from the urolithin C produced by the production method described in the section “2-1-5. Urolithin C production method (third aspect)”.

The steps of the “2-1-5. Urolithin C production method (third aspect)” of the present aspect are described below. In this case, Step (I) described above corresponds to Step (II) below.

That is, the present aspect is a method for producing urolithin A, the method including the following Steps (I) and (II):

Step (I): bringing the transformant described in the section “2-1-3. Genetic engineering aspects”, wherein host of the transformant is a microorganism having an ability to produce urolithin C from ellagic acid, into contact with ellagic acid, so as to produce urolithin C from the ellagic acid.

Step (II): allowing a microorganism having the ability to produce urolithin A from urolithin C to produce urolithin A from the urolithin C.

### (Step (I))

Regarding Step (I), the details described in the aforementioned “2-1-5. Urolithin C production method (third aspect)” are incorporated by reference.

### (Step (II))

In Step (II), a microorganism having the ability to produce urolithin A from urolithin C is caused to produce urolithin A from urolithin C. Regarding Step (II), the details described in the aforementioned “1-3-3. Urolithin A production method (first aspect)” section are incorporated by reference.

Steps (I) and (II) may be implemented in the same system.

The above “implemented in the same system” means that with regard to the series in which the urolithin C produced in Step (I) is used as is as the urolithin C in Step (II) to produce the urolithin A in Step (II), the series is continuously implemented in the same system. That is, for example, this series does not include a step of separating and/or purifying the urolithin C produced in Step (I). Note that when microorganisms are used in each step, the microorganisms in each step may be the same or different.

The present aspect may include other steps. The details of these steps are the same as the details described in the section “1-3-1. Urolithin C production method (first aspect)”.

### <2-2-1. Lactonase>

Another aspect of the present disclosure is a lactonase.

Lactonases exhibit activity of catalyzing a reaction in which at least one of the two ester bonds present in ellagic acid is hydrolyzed. For example, a lactonase exhibits activity of catalyzing a reaction in which urolithin M5 is produced from ellagic acid.

The lactonase according to the present aspect is preferably derived from a microorganism belonging to the genus Gordonibacter.

The lactonase is more preferably one or more selected from the group consisting of: a microorganism belonging to Gordonibacter urolithinfaciens; a microorganism belonging to Gordonibacter pamelaeae; and a microorganism belonging to Gordonibacter faecihominis.

The microorganism belonging to Gordonibacter urolithinfaciens is preferably of the Gordonibacter urolithinfaciens DSM 27213 strain.

The microorganism belonging to Gordonibacter pamelaeae is preferably of the DSM 19378 strain.

The microorganism belonging to Gordonibacter faecihominis is preferably of the JCM 16058 strain.

The amino acid sequence of the lactonase of the Gordonibacter urolithinfaciens DSM 27213 strain is the sequence represented by SEQ ID NO: 29. Furthermore, the base sequence of the gene encoding the lactonase thereof is the sequence represented by SEQ ID NO: 30.

The amino acid sequence of the lactonase derived from the Gordonibacter pamelaeae DSM 19378 strain is the sequence represented by SEQ ID NO: 31. Moreover, the base sequence of the gene encoding the lactonase thereof is the sequence represented by SEQ ID NO: 32.

Therefore, the lactonase according to the present aspect is preferably a protein (lactonase) containing the amino acid sequence represented by SEQ ID NO: 29 or containing the amino acid sequence represented by SEQ ID NO: 31.

Also, the lactonase derived from the Gordonibacter urolithinfaciens DSM 27213 strain may be a protein formed from amino acids, the protein thereof being such that in the amino acid sequence represented by SEQ ID NO: 29, one to a plurality of amino acids are substituted or deleted, or one to a plurality of amino acids are inserted or added, as long as the protein exhibits lactonase activity. One to a plurality means preferably from 1 to 36, more preferably from 1 to 35, even more preferably from 1 to 20, yet even more preferably from 1 to 10, and particularly preferably from 1 to 5, and the same applies to a case in which the amino acids are added to the N-terminal side and/or the C-terminal side.

The details of an aspect of substitution are the same as the details described in the section “1-1-1 (A). Enzyme that dehydroxylates the hydroxyl group at the 4-position of urolithins” above.

Also, the lactonase derived from the Gordonibacter urolithinfaciens DSM 27213 strain may be a protein having homology of 80% or greater, preferably 90% or greater, more preferably 95% or greater, even more preferably 97% or greater, and particularly preferably 99% or greater in relation to the entire length of the amino acid sequence represented by SEQ ID NO: 29, as long as the protein exhibits lactonase activity.

Furthermore, the lactonase derived from the Gordonibacter urolithinfaciens DSM 27213 strain may be a protein encoded by a polynucleotide that hybridizes under stringent conditions with a base sequence represented by SEQ ID NO: 30, as long as the protein exhibits lactonase activity. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize.

The details regarding substitutions of the amino acids, protein homology, stringent conditions, and the like are also applicable to an ellagic acid transporter derived from the Gordonibacter pamelaeae DSM 19378 strain. However, with regard to the ellagic acid transporter derived from the Gordonibacter pamelaeae DSM 19378 strain, the “one to a plurality” is preferably from 1 to 36, more preferably from 1 to 35, even more preferably from 1 to 20, yet even more preferably from 1 to 10, and particularly preferably from 1 to 5.

The lactonase activity from the lactonase according to the present aspect can be evaluated, for example, as follows. A reaction solution containing 20 mM of a potassium phosphate buffer (pH 6.5), 1 mg/mL of ellagic acid, and a lactonase is prepared and reacted for 3 hours at 37 °C, after which the urolithin M5 that is produced is measured through high-performance liquid chromatography (HPLC) under the following conditions.

### <HPLC Conditions>

Column: Cosmosil 5C18-AR-II (4.6 mm inner diameter×150 mm length)
Eluent: ultrapure water (Milli Q water)/acetonitrile/formic acid=80/20/1
Flow rate: 1 mL/min
Column temperature: 40 °C
Detection: UV (349 nm)

### <2-2-2. Polynucleotide Containing a Gene Encoding a Lactonase>

Another aspect of the present disclosure is a polynucleotide containing a base sequence represented by SEQ ID NO: 30 or containing a base sequence represented by SEQ ID NO: 32. The details of each base sequence are as previously described.

The base sequence of a gene encoding the lactonase derived from the Gordonibacter urolithinfaciens DSM 27213 strain may be a base sequence that hybridizes with the base sequence represented by SEQ ID NO: 30 under stringent conditions, as long as the protein encoded by the gene thereof exhibits lactonase activity. “Stringent conditions” include, for example, conditions in which polynucleotides having homology of, for example, 80% or greater, preferably 90% or greater, more preferably 95% or greater, even more preferably 97% or greater, and particularly preferably 99% or greater hybridize, while polynucleotides having homology less than this do not hybridize.

This matter is also applicable to a base sequence of a gene encoding lactonase derived from the Gordonibacter pamelaeae DSM 19378 strain.

### <2-2-3. Genetic Engineering Aspects>

Other aspects of the present disclosure include a recombinant vector containing the polynucleotide described in the section “2-2-2. Polynucleotide containing a gene encoding a lactonase”, a transformant in which the polynucleotide described in the section “2-2-2. Polynucleotide containing a gene encoding lactonase” is expressively retained, or in which the vector is expressively retained, and a method for producing a protein encoded by the polynucleotide, the method including a step of culturing the transformant. The protein is the “lactonase”.

The details of these aspects are similar to the details described in the “1-1-3 (A). Genetic engineering aspects” section.

Also, the transformant is one having an activity of catalyzing a reaction in which at least one of the two ester bonds present in ellagic acid is hydrolyzed. Accordingly, a preferred aspect is also a method of promoting the reaction that hydrolyzes at least one of the two ester bonds present in the ellagic acid, the method including the following Step (I):

Step (I): bringing the transformant into contact with ellagic acid.

The details of the transformant are similar to the details described in the previously mentioned “1-1-3 (A). Genetic engineering aspects” section. Furthermore, an example of an “aspect in which the transformant is caused into contact with ellagic acid” is an aspect in which the microorganism that produces the enzyme described in (i) or the microorganism that produces the protein described in (ii) are used in the method described in the section “1-2-1 (A). Method for dehydroxylating the hydroxyl group at the 4-position of urolithins”.

With the transformant in which the polynucleotide described in the section “2-2-2. Polynucleotide containing a gene encoding lactonase” is expressively retained, or in which the vector is expressively retained, it is preferable that expression of the polynucleotide described in the section “2-1-2. Polynucleotide containing a gene encoding an ellagic acid transporter” also be possible. That is, with the transformant, it is preferable that the lactonase and the ellagic acid transporter are co-expressed.

### <2-2-4. Urolithin M5 Production Method (Second Aspect)>

Another aspect of the present disclosure is a method (second aspect) for producing urolithin M5.

The present aspect is a method for producing urolithin M5, the method including the following Step (I):

Step (I): bringing the transformant described in the section “2-2-3. Genetic engineering aspects” into contact with ellagic acid, so as to produce urolithin M5 from the ellagic acid.

### (Step (I))

In Step (I), the transformant described in the section “2-2-3. Genetic engineering aspects” is caused into contact with ellagic acid, so as to produce urolithin M5 from the ellagic acid.

The transformant described in the section “2-2-3. Genetic engineering aspects” is a microorganism having the ability to produce urolithin M5 from ellagic acid in order to express lactonase.

Furthermore, an example of an aspect of contact between the transformant and ellagic acid is an aspect in which the microorganism that produces the enzyme described in (i) or the microorganism that produces the protein described in (ii) are used in the method described in the section “1-2-1 (A). Method for dehydroxylating the hydroxyl group at the 4-position of urolithins”.

The present aspect may include other steps. The details of these steps are the same as the details described in the section “1-3-1. Urolithin C production method (first aspect)”. Also, for example, urolithins may be produced in accordance with the method for producing urolithins described in the present specification, using the urolithin M5 that is produced. In addition, the present aspect may be implemented with a step of producing urolithin M5 from an ellagic acid in the method for producing urolithins described herein.

### <2-2-5. Urolithin C Production Method (Fourth Aspect)>

Another aspect of the present disclosure is a method for producing urolithin C (fourth aspect).

The present aspect is a method for producing urolithin C, the method including the following Step (I):

Step (I): bringing the transformant described in the section “2-2-3. Genetic engineering aspects”, wherein host of the transformant is a microorganism having an ability to produce urolithin C from urolithin M5, into contact with ellagic acid, so as to produce urolithin M5 from the ellagic acid, and then produce urolithin C from the urolithin M5.

### (Step (I))

In Step (I), the transformant described in the section “2-2-3. Genetic engineering aspects”, wherein host of the transformant is a microorganism having an ability to produce urolithin C from urolithin M5, is brought into contact with ellagic acid, so as to produce urolithin M5 from the ellagic acid, and then produce urolithin C from the urolithin M5.

The transformant described in the section “2-2-3. Genetic engineering aspects” is a microorganism having the ability to produce urolithin M5 from ellagic acid in order to express lactonase.

Also, the microorganism, which is the host and has the ability to produce urolithin C from urolithin M5, may be E. coli, which is commonly used as a host for transformation, but other examples can include the microorganism described in the section “1-2-1 (B). Method for dehydroxylating the hydroxyl group at the 10-position of urolithins” above.

Furthermore, an example of an aspect of contact between the transformant and ellagic acid is an aspect in which the microorganism that produces the enzyme described in (i) or the microorganism that produces the protein described in (ii) are used in the method described in the section “1-2-1 (A). Method for dehydroxylating the hydroxyl group at the 4-position of urolithins”.

The present aspect may include other steps. The details of these steps are the same as the details described in the section “1-3-1. Urolithin C production method (first aspect)”. Also, for example, urolithins may be produced in accordance with the method for producing urolithins described in the present specification, using the urolithin C that is produced. In addition, in the method for producing urolithins described herein, the present aspect may be implemented with a step of producing urolithin M5 from ellagic acid, and a step of producing urolithin C from the urolithin M5.

### <2-2-6. Urolithin A Production Method (Fourth Aspect)>

Another aspect of the present disclosure is a method (fourth aspect) for producing urolithin A.

The present aspect is a method for producing urolithin A, the method including the following Step (I) and (II):

Step (I): bringing the transformant described in the section “2-2-3. Genetic engineering aspects”, wherein host of the transformant is a microorganism having an ability to produce urolithin C from urolithin M5, into contact with ellagic acid, so as to produce urolithin M5 from the ellagic acid, and then produce urolithin C from the urolithin M5.

Step (II): allowing a microorganism having the ability to produce urolithin A from urolithin C to produce urolithin A from the urolithin C.

### (Step (I))

Regarding Step (I), Step (I) described in the section “2-2-5. Urolithin C production method (fourth aspect)” is incorporated by reference.

### (Step (II))

In Step (II), a microorganism having the ability to produce urolithin A from urolithin C is allowed to produce urolithin A from urolithin C. Regarding Step (II), the details described in the aforementioned “1-3-3. Urolithin A production method (first aspect)” section are incorporated by reference.

Steps (I) and (II) may be implemented in the same system.

The above “implemented in the same system” means that the series in which the urolithin C produced in Step (I) is used as is as the urolithin C in Step (II) to produce the urolithin A in Step (II) is continuously implemented in the same system. That is, for example, this series does not include a step of separating and/or purifying the urolithin C produced in Step (I). Note that when microorganisms are used in each step, the microorganism in each step may be the same or different.

The present aspect may include other steps. The details of these steps are the same as the details described in the section “1-3-1. Urolithin C production method (first aspect)”.

### EXAMPLES

Examples are described below, but none of the examples are interpreted to be limiting.

### Experimental Example 1: Preparation of Bacterial Cells for Proteome Analysis

A 10 mL ABB culture medium (ANAEROBE BASAL BROTH, Oxoid Limited, Code CM0957) containing 0.1% of ellagic acid (Sigma-Aldrich Corporation) and a 10 mL ABB culture medium (ANAEROBE BASAL BROTH, Oxoid Limited, Code CM0957) not containing the ellagic acid were both inoculated with the Gordonibacter urolithinfaciens DSM 27213 strain, and then cultured for 16 days at 37 °C in an anaerobic gas (N₂/H₂/CO₂=80:10:10) environment.

The culturing solutions were centrifuged to collect bacterial cells, and the bacterial cells were then used in proteome analysis.

### Experimental Example 2: Proteome Analysis

The bacterial cells prepared in Experimental Example 1 were lysed using a lysate (7 M urea, 2 M thiourea, 2% CHAPS (3-[(3-cholamidopropyl) dimethylammonio]propanesulfonate), 10 mM DTT (dithiothreitol), and 50 mM tris-hydrochloric acid buffer (pH 7.0)), and were then subjected to proteome analysis through the method described in AMB Express, 2:37 (2012).

As a result, in comparison to the case in which culturing was implemented with the culture medium not containing ellagic acid, in the case in which culturing was implemented with a culture medium containing ellagic acid, proteins with significantly increased expression, that is, proteins for which expression was induced by the ellagic acid, were detected. The proteins were then analyzed on the basis of genomic information, and CD2938, CD2940, CD2941, CD2942 and CD2943, as well as CD2949, CD2950 and CD2952 were identified. GENETYX (Genetyx Corporation) was used was for analysis of gene information such as homology analysis.

### Experimental Example 3: Protein Function Prediction

The functions of the amino acid sequences of CD2938, CD2940, CD2941, CD2942 and CD2943 were predicted through a homology search with known proteins. The results were as follows.

CD2938 was presumed to be “anaerobic dehydrogenase having Se-Cys (selenocysteine) and Mo (molybdenum)-pterin bonding regions”.

CD2940 was presumed to be a “hydrogenase component having an Fe—S (iron-sulfur) cluster”.

CD2941 exhibited homology with “cations and various drug delivery pumps”.

It is presumed that these CD2938, CD2940, and CD2941 may be sub-units that constitute an enzyme that dehydroxylates the hydroxyl group at the 4-position of urolithins. These genes were named GuuroA1, GuuroA2, and GuuroA3. In addition, these genes are genes encoding for GuUroA1, GuUroA2, and GuUroA3, respectively.

CD2942 was presumed to be a “metal-dependent hydrolase”, and the gene thereof was named uroH. This gene encodes a lactonase. The lactonase thereof exhibits activity of catalyzing a reaction in which at least one of the two ester bonds present in ellagic acid is hydrolyzed.

The CD2943 was presumed to be a “permease belonging to the major facilitator superfamily” and was named uroT. In the biosynthesis pathway of urolithins, it is speculated that CD2943 functions as a transporter for uptake of ellagic acid into the bacterial cells.

Similar to CD2938 and CD2940, CD2949 and CD2950 are presumed to be “anaerobic dehydrogenase having Se-Cys (selenocysteine) and Mo (molybdenum)-pterin bonding regions” and a “hydrogenase component having an Fe—S (iron-sulfur) cluster”, respectively.

The CD2952 is presumed to be related to “heme utilization or adsorption”.

Thus, it is presumed that CD2949, CD2950, and CD2952 may be sub-units that constitute an enzyme that dehydroxylates the hydroxyl group at the 10-position of urolithins. These genes were named GuuroB1, GuuroB2, and GuuroB3. In addition, these genes encode GuUroB1, GuUroB2, and GuUroB3, respectively.

The homology between CD2938 and CD2949, the homology between CD2940 and CD2950, and the homology between CD2941 and CD2951 were 42%, 50%, and 17%, respectively, and thus the possibility that “CD2938 and CD2940”, “CD2949 and CD2950”, “CD2938 and CD2940 and CD2941” and “CD2949 and CD2950 and CD2952” function as complexes was suggested.

Experimental Example 4: Purification of enzyme that dehydroxylates the hydroxyl group at the 4-position of urolithins (Culturing of microorganisms) As a pre-culture, 20 mL of a modified GAM culture medium (Nihon Pharmaceutical Co., Ltd.) containing 0.1 mg/mL of ellagic acid was added to a 100 mL Erlenmeyer flask and then inoculated with the Gordonibacter urolithinfaciens DSM 27213 strain, after which the mixture was shaken at 80 rpm and cultured in an anaerobic chamber at 37 °C for 5 to 6 days.

A 2 L Erlenmeyer flask containing 1.75 L of the same culture medium was inoculated with a pre-culturing solution, and then shaken at 80 rpm and cultured in an anaerobic chamber for 7 to 9 days at 37 °C.

The obtained culturing solution was centrifuged (8,000 rpm, 15 min, 4 °C) to prepare bacterial cells as a precipitated fraction.

### (Preparation of Ultra-Centrifuged Supernatant)

The obtained bacterial cells were suspended in a 50 mM potassium phosphate buffer (KPB, pH 6.5), and the bacteria were crushed using a Kubota Insonator Model 201M (Kubota Corporation). The ultrasonic crushed liquid was centrifuged at 8,500 rpm for 30 minutes at 4 °C, and the supernatant was then centrifuged for 60 minutes at 20,000 g to obtain a supernatant, which was then used as a cell-free extract. The obtained cell-free extract was then ultra-centrifuged at 100,000 g for 110 minutes to obtain a supernatant fraction.

### (Enzyme Purification (Mono Q First Time))

The obtained ultra-centrifuged supernatant was subjected to anion exchange chromatography using a Mono Q 5/50 column (GE Healthcare Japan Corp.).
Eluent A: 50 mM KPB (pH 6.5)
Eluent B: 50 mM KPB (pH 6.5) and 1 M NaCl
Flow rate: 0.8 mL/min.

Gradient elution from the eluent A to the eluent B was implemented, the active fraction of the enzyme was collected and concentrated by gel filtration, and the resulting product was used as a 1st Mono Q fraction.

### (Enzyme Purification (Superdex 200))

The obtained 1st Mono Q fraction was subjected to gel filtration chromatography using the Superdex 200 Increase 10/300 GL (Ge Healthcare Japan Corp.).
Eluent: 50 mM KPB (pH 6.5) and 150 mM NaCl
Flow rate: 0.25 mL/min

The active fraction of the enzyme was collected and concentrated by ultra-filtration, and the obtained product was used as a Superdex fraction.

### (Enzyme Purification (Mono Q Second Time))

The resulting Superdex fraction was purified again by anion exchange chromatography through Mono Q.
Eluent A: 50 mM KPB (pH 6.5)
Eluent B: 50 mM KBP (pH 6.5) and 1 M NaCl
Flow rate: 0.8 mL/min.

Gradient elution from the eluent A to the eluent B was implemented, the active fraction of the enzyme was collected and concentrated by gel filtration, and the resulting product was used as a 2nd Mono Q fraction.

The purification process of the enzyme thus far is summarized in Table 1.

### Experimental Example 5: Measuring the Activity of the Enzyme that Dehydroxylates the Hydroxyl Group at the 4-Position of Urolithins

The activity of dehydroxylating the hydroxyl group at the 4-position of urolithins was measured for each fraction obtained in Experimental Example 4. Urolithin M5 was used as the urolithin having a hydroxyl group at the 4-position. When the hydroxyl group at the 4-position was dehydroxylated, urolithin M6 was produced.

An amount of 30 μL of a reaction solution for measuring enzyme activity was inserted into a 200 μL tube for PCR with the reaction solution containing 1 mg/mL of urolithin M5, 10 mM of NADPH (reduced nicotinamide adenine dinucleotide phosphate), 0.2 mM of FAD (flavin adenine dinucleotide), 10 mM of MV (methyl viologen), 50 mM of KPB (potassium phosphate buffer, pH 6.5) and each fraction. The gas phase was set to the gas phase in the anaerobic chamber (Vinyl Anaerobic Chamber available from Coy Labs), and the materials were reacted at 37 °C for 90 minutes. Next, after the reaction was completed, 60 μL of N,N-dimethylacetamide containing 1% formic acid was added to the reaction solution and mixed, after which the supernatant of centrifugation was analyzed by HPLC.

HPLC was carried out under the following conditions, and the resulting urolithin M6 was quantitatively determined (detection wavelength: 348 nm).

### <HPLC Conditions>

Column: COSMOSIL 5C18-ARII (4.6 mm inner diameter×150 mm length, available from Nacalai Tesque, Inc.)
Eluent: ultrapure water (Milli Q water)/acetonitrile (CH₃CN)/formic acid=80/20/1
Flow rate: 1.0 mL/min
Temperature: 40 °C
Detection: PDA (photodiode array)

Enzyme activity 1 U was the amount of enzyme for catalyzing the production of 1 pmol of urolithin M6 in 90 minutes.

The results are shown in Table 1.

TABLE 1

| | | | Enzyme | Specific | Purification |
|---|---|---|---|---|---|
| | Volume | Protein | Activity | Activity | Scale |
| Step | (mL) | (mg) | (U) | (U/mg) | Factor |
| Ultra-centrifuged | 10.0 | 8.52 | 0.542 | 0.0637 | 1 |
| supernatant | | | | | |
| 1st Mono Q | 1.50 | 0.77 | 0.085 | 0.111 | 1.74 |
| Superdex 200 | 1.35 | 0.0480 | 0.00541 | 0.113 | 1.77 |
| 2nd Mono Q | 0.150 | 0.00192 | 0.000223 | 0.116 | 1.83 |

Experimental Example 6: Native-PAGE of Enzyme that Dehydroxylates a Hydroxyl Group at the 4-Position of Urolithins and of an Enzyme that Dehydroxylates the Hydroxyl Group at the 10-Position of the Urolithins

The enzyme fractions purified thus far were subjected to unmodified polyacrylamide electrophoresis (Native-PAGE) using a 5 to 20% gradient gel (PAGEL, NPG-520 L, ATTO Corp.), the resulting bands were semi-dry blotted with a PVDF membrane (Immobilon-P Transfer membrane, available from Millipore) using HorizeBLOT AE6677G (available from ATTO Corp.), each band was cut out, and the sequence was analyzed using an amino acid sequencer.

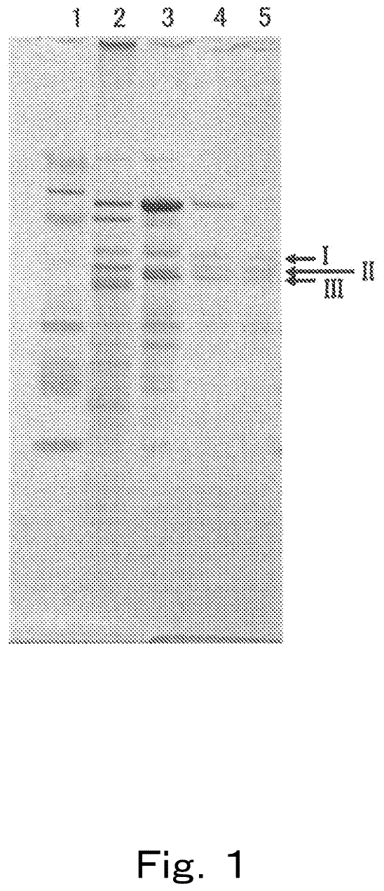

The results are illustrated in FIG. 1. In FIG. 1, 1 is a molecular weight marker lane, 2 is the lane of the ultra-centrifuged supernatant fraction, 3 is the lane of the first Mono Q fraction, 4 is the lane of the Superdex fraction, and 5 is lane of second Mono Q fraction.

From the amino acid sequence of band I, band I is presumed to be formate-tetrahy drofolate ligase.

Two types of amino acid sequences are obtained in band II, and these are presumed to be GuUroB1 (protein corresponding to the gene GuuroB1) and GuUroB2 (protein corresponding to the gene GuuroB2), respectively.

Two types of amino acid sequences are also obtained in band III, and these are presumed to be GuUroA1 (protein corresponding to the gene GuuroA1) and GuUroA2 (protein corresponding to the gene GuuroA2).

### Experimental Example 7: SDS-PAGE of Enzyme that Dehydroxylates a Hydroxyl Group at the 4-Position of Urolithins and of an Enzyme that Dehydroxylates the Hydroxyl Group at the 10-Position of the Urolithins

The enzyme fractions purified thus far were subjected to sodium dodecyl sulphate-polyacrylamide gel electrophoresis (SDS-PAGE) using a 5 to 20% gradient gel (e-PAGEL, E-R520L, available from ATTO Corp.), and the molecular weight was evaluated. The Protein Molecular Weight Marker (Broad) available from Takara Bio Inc. was used as the molecular weight marker.

The resulting bands were semi-dry blotted with a PVDF membrane (Immobilon-P Transfer membrane, available from Millipore) using HorizeBLOT AE6677G (available from ATTO Corp.), each band was cut out, and the sequence was analyzed using an amino acid sequencer.

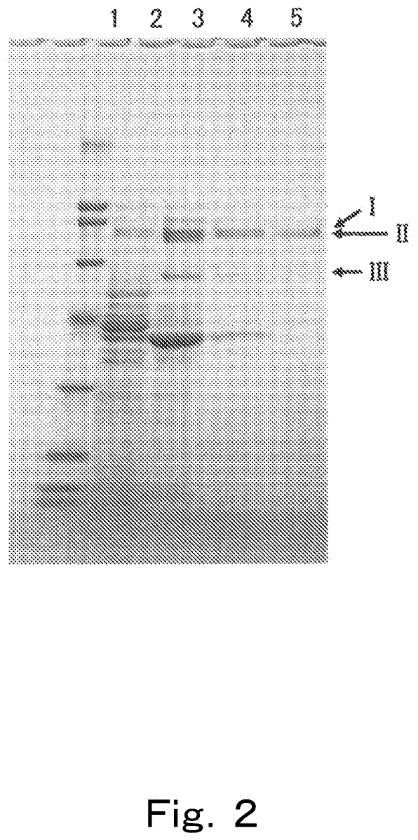

The results are illustrated in FIG. 2. In FIG. 2, 1 is a molecular weight marker lane, 2 is the lane of the ultra-centrifuged supernatant fraction, 3 is the lane of the first Mono Q fraction, 4 is the lane of the Superdex fraction, and 5 is lane of the second Mono Q fraction.

The amino acid sequences of band I and band II obtained in the 2nd Mono Q fraction in lane 5 were analyzed together, and as a result, two types of amino acid sequences were obtained. These amino acid sequences were identified as GuUroA1 (protein corresponding to the gene GuuroA1) and GuUroB1 (protein corresponding to the gene GuuroB1), respectively. The molecular mass of each band was assumed to be 90.0 kDa and 87.0 kDa, respectively.

From the amino acid sequence of band III, band III is presumed to be formate-tetrahy drofolate ligase.

### Experimental Example 8: Effect of Cofactors on the Activity of Enzymes that Dehydroxylate the Hydroxyl Group at the 4-Position of Urolithins

Cofactors of the concentrations listed in Table 2 were added to a reaction solution obtained by removing NADPH, FAD, and MV from the reaction solution for measuring enzyme activity described in Experimental Example 5, and the mixture was reacted, after which the produced urolithin M6 was quantitatively determined. The 1st Mono Q fraction was used as the enzyme. With the amount of urolithin M6 produced under the conditions indicated for No. 12 in Table 2 (10 mM MV, 10 mM NADPH, 0.2 mM FAD) considered to be 100%, the amount of urolithin M6 produced under each condition was expressed as a relative activity.

The results are summarized as shown in Table 2 below.

Activity was confirmed in cases where NADH, NADPH, FMN, or MV was added alone, as compared to a case in which none of these were added.

A synergistic increase in activity was also confirmed in cases in which NADH or NADPH was present along with the presence of MV.

A significant increase in activity was confirmed in cases in which FAD or FMN was present along with the presence of MV and NADPH.

The optimal condition was a combination of 10 mM MV, 10 mM NADPH, and 0.2 mM FAD.

In the following experimental examples, this condition was used as a standard activity measurement condition.

TABLE 2

| | | Cofactors (mM) | | | | | Relative |
|---|---|---|---|---|---|---|---|
| No. | Enzyme | MV | NADH | NADPH | FAD | FMN | Activity |
| 1 | − | — | — | — | — | — | 0.0% |
| 2 | + | — | — | — | — | — | 0.0% |
| 3 | + | — | 10 | — | — | — | 2.4% |
| 4 | + | — | — | 10 | — | — | 0.8% |
| 5 | + | — | — | — | 0.2 | | 0.0% |
| 6 | + | — | — | — | | 0.2 | 0.0% |
| 7 | + | 10 | — | — | — | — | 0.8% |
| 8 | + | 10 | 10 | — | — | — | 12.0% |
| 9 | + | 10 | — | 10 | — | — | 24.8% |
| 10 | + | 10 | — | — | 0.2 | — | 0.8% |
| 11 | + | 10 | — | — | — | 0.2 | 0.0% |
| 12 | + | 10 | — | 10 | 0.2 | — | 100% |
| 13 | + | 10 | — | 10 | — | 0.2 | 94.4% |

### Experimental Example 9: Temperature Dependency of Enzymes that Dehydroxylate the Hydroxyl Group at the 4-Position of Urolithins

Under the standard activity measurement conditions described in Experimental Example 8, the reaction temperature was changed, and the activity was measured.

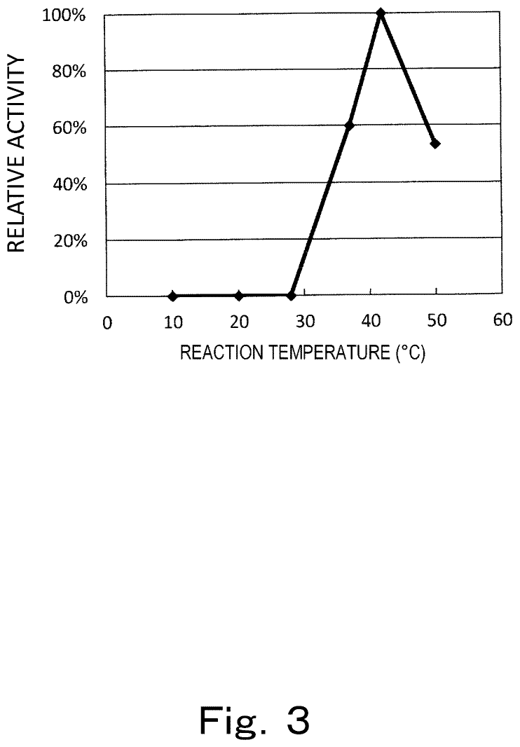

The results are illustrated in FIG. 3. The optimum temperature was 42 °C, and the activity at that time was considered to be 100%. In the temperature range of 37 to 50 °C, an activity of 50% or greater was exhibited.

### Experimental Example 10: PH Dependency of Enzymes that Dehydroxylate the Hydroxyl Group at the 4-Position of Urolithins

Under the standard activity measurement conditions described in Experimental Example 8, the pH was changed using the following buffer, and the activity was measured.
Citric acid buffer: 50 mM citric acid-sodium citrate buffer
Acetic acid buffer: 50 mM acetic acid-sodium acetate buffer
Potassium phosphate buffer: 50 mM K₂HPO₄—KH₂PO₄ buffer
Tris-HCl buffer: 50 mM Tris-hydrochloride buffer
Carbonate buffer: 50 mM Na₂CO₃—NaHCO₃ buffer
Sodium phosphate buffer: 50 mM Na₃PO₄—Na₂HPO₄ buffer

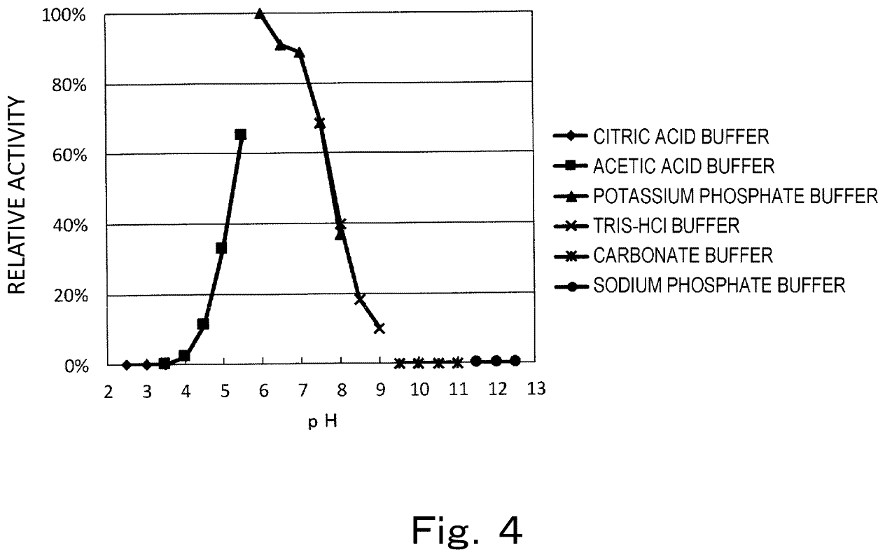

The results are illustrated in FIG. 4. The optimum pH was 6.0, and activity of 80% or more of the activity in optimum conditions was exhibited in a pH range of from 6.0 to 7.0.

### Experimental Example 11: Temperature Stability of Enzymes that Dehydroxylate the Hydroxyl Group at the 4-Position of Urolithins

The enzyme was maintained at a temperature of from 4 to 60 °C for 12 hours, after which the activity was measured at the standard activity measurement conditions described in Experimental Example 8.

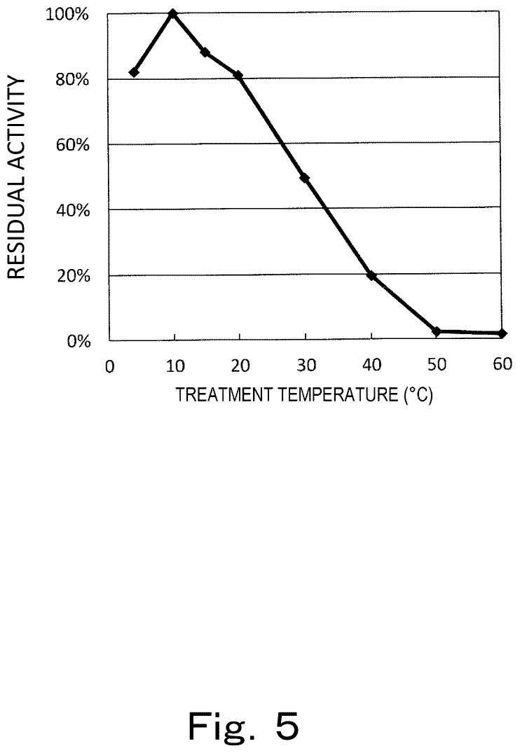

The results are illustrated in FIG. 5. The optimum temperature was 10 °C, and when the residual activity at 10 °C was considered to be 100%, a residual activity of 80% or greater was exhibited in a temperature range of from 4 to 20 °C.

### Experimental Example 12: PH Stability of Enzymes that Dehydroxylate the Hydroxyl Group at the 4-Position of Urolithins

An amount of 90 μL of each of the following buffers at a molar concentration of 100 mM was added to 10 μL of the 1st Mono Q fraction as an enzyme, and the mixture was concentrated 10-fold through ultra-filtration, after which the resulting product was held at 4 °C for 12 hours. Subsequently, 60 μL of 50 mM KPB (pH 6.5) was added to restore the pH, and then the activity was measured under the standard activity measurement conditions described in Experimental Example 8.
Citric acid buffer: 50 mM citric acid-sodium citrate buffer
Acetic acid buffer: 50 mM acetic acid-sodium acetate buffer
Potassium phosphate buffer: 50 mM K₂HPO₄—KH₂PO₄ buffer
Tris-HCl buffer: 50 mM Tris-hydrochloride buffer
Carbonate buffer: 50 mM Na₂CO₃—NaHCO₃ buffer
Sodium phosphate buffer: 50 mM Na₃PO₄—Na₂HPO₄ buffer

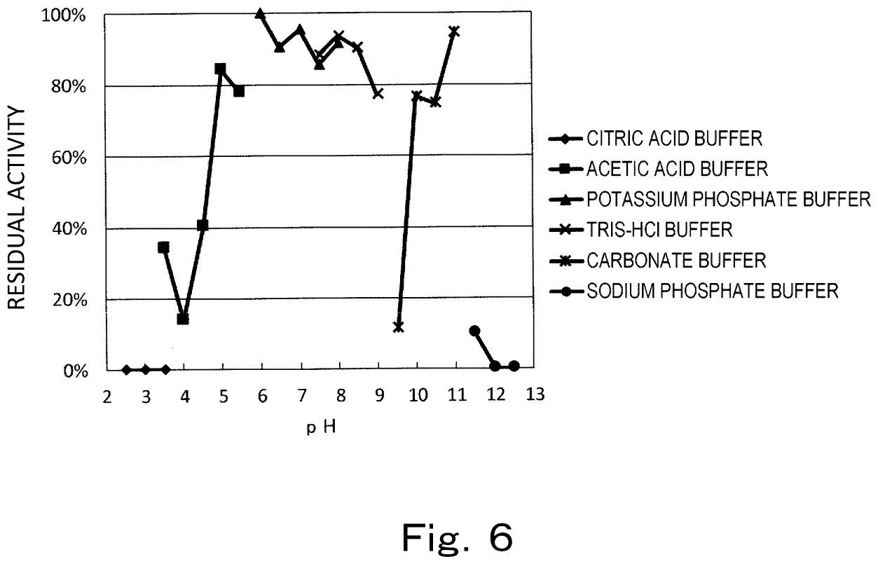

The results are illustrated in FIG. 6. The most stable pH was 6.0 (potassium phosphate buffer). Thus, the activity at a pH of 6.0 was considered to be 100%, and on the basis thereof, the residual activity was 75% or greater in a pH range of from 5.0 to 9.0. The residual activity was also 75% or greater in a pH range of from 10.0 to 11.0 (carbonate buffer).

### Experimental Example 13: Construction of a Plasmid Expressing an Ellagic Acid Transporter

Genomic DNA was prepared from the Gordonibacter urolithinfaciens DSM 27213 strain. This genomic DNA was then used as a template, and the ellagic acid transporter gene (uroT) was amplified by PCR using the following primer set and then inserted into the BamHI and EcoRI sites of the expression vector pRSFDuet-1 to construct pRSFDuet-1_uroT.

(SEQ ID NO: 33)
5′-AAAGGATCCATGAGCAATCCGAATGCCGCCGTAGCGGCAA
GC-3′
(SEQ ID NO: 34)
5′-GTTGAATTCTTAGTGCTGTGCGGCTGCCTCGTCTGCGGGTT
T-3′

### Experimental Example 14: Construction of a Plasmid Expressing Lactonase

Genomic DNA was prepared from the Gordonibacter urolithinfaciens DSM 27213 strain according to a routine method. This genomic DNA was then used as a template, and the lactonase gene (uroH) was amplified by PCR using the primer set below and then inserted into the BamHI and EcoRI sites of the expression vector pET-21b(+) to construct pET21b_UroH.

(SEQ ID NO: 35)
5′-GCCGGATCCCATGGCAGACAACAAGGTCATCGACATCAAC
ATG-3′
(SEQ ID NO: 36)
5′-TATGAATTCCTACAGGTTGAACAGCTTCGCCGCGTTGCCGC
C-3′

### Experimental Example 15: Expression of Ellagic Acid Transporter in E. coli

The E. coli Rosetta2 (DE3) strain was transformed with pRSFDuet-1_uroT through the Ca method.

The obtained transformed strain was shaken and cultured at 37 °C for 4 hours in an LB culture medium containing 5 mL of 34 μg/mL chloramphenicol and/or 39 μg/mL kanamycin. IPTG was added to the culture solution to a molar concentration of 1 mM and then subjected to induced culturing at 30 °C for 4 hours.

The obtained culturing solution was centrifuged and washed with 0.85% NaCl to prepare wet bacterial cells.

### Experimental Example 16: Expression of Lactonase and Ellagic Acid Transporter with Respect to E. coli

The E. coli Rosetta2 (DE3) strain was transformed with pET21b_UroH and pRSFDuet-1_uroT through the Ca method.

The obtained transformed strain was shaken and cultured at 37 °C for 4 hours in an LB culture medium containing 5 mL of 34 μg/mL chloramphenicol and/or 39 μg/mL kanamycin. IPTG was added to the culture solution to a molar concentration of 1 mM and then subjected to induced culturing at 30 °C for 4 hours.

The obtained culturing solution was centrifuged and washed with 0.85% NaCl to prepare wet bacterial cells.

### Experimental Example 17: Expression of Lactonase with Respect to E. coli

The E. coli Rosetta2 (DE3) strain was transformed with pET21b_UroH through the Ca method.

The obtained transformed strain was shaken and cultured at 37 °C for 4 hours in an LB culture medium containing 5 mL of 34 μg/mL chloramphenicol and/or 39 μg/mL kanamycin. IPTG was added to the culture solution to a molar concentration of 1 mM and then subjected to induced culturing at 30 °C for 4 hours.

The obtained culturing solution was centrifuged and washed with 0.85% NaCl to prepare wet bacterial cells.

### Experimental Example 18-1: Production of Urolithin M5 from Ellagic Acid Using a Transformed Strain

The bacterial cells contained in 3 mL of each of the culturing solutions obtained in Experimental Example 15, Experimental Example 16, and Experimental Example 17 were reacted at 37 °C for 4 hours in a 100 mM KPB (pH 6.5) buffer containing 5 mg of ellagic acid, and the amount of urolithin M5 that was produced was determined for each case. Note that the quantitative determination of the urolithin M5 was implemented in the same manner as in Experimental Example 5.

The results are shown in Table 3. Note that the scale factor in Table 3 is a relative value with respect to the production amount of urolithin M5 when only the uroH gene was introduced (Experimental Example 17).

When only the uroT gene was introduced (Experimental Example 15), no urolithin M5 was produced.

When only the uroH gene was introduced (Experimental Example 17), urolithin M5 was produced at a molar concentration of 0.960 mM.

When the uroH gene and the uroT gene were introduced (Experimental Example 16), urolithin M5 was produced at a molar concentration of 1.22 mM, and the production amount of urolithin M5 increased 1.3 times in comparison to the case in which only the uroH gene was introduced (Experimental Example 17).

TABLE 3

| Expression Genes | Urolithin M5 (mM) | Scale Factor |
|---|---|---|
| uroH | 0.960 | 1.0 |
| uroT | 0.000 | 0.0 |
| uroH and uroT | 1.220 | 1.3 |

### Experimental Example 18-2: Production of Urolithin M5 from Ellagic Acid Using a Deposited Strain

Ellagic acid was added to an ABB culture medium (available from Oxoid Limited) with the final concentration of ellagic acid being 1 g/L, after which the mixture was heated and sterilized, the gas phase was purged with a gas of N₂:CO₂:H₂ (80%/10%/10%), and the resulting product was used as a basal culture medium. The basal culture medium was inoculated with the Gordonibacter faecihominis JCM 16058 strain and anaerobically cultured at 37 °C for 14 days.

The amount of urolithins in the culture solution after cultivation was quantitatively determined by the following method.

Urolithins were extracted from 5 mL of the culture solution with an equal amount of ethyl acetate, and the resulting ethyl acetate phase was concentrated under reduced pressure and dried to obtain a solid. The dry solid obtained in this manner was re-dissolved in 0.5 mL of methanol, and the amount of urolithins was quantitatively analyzed through HPLC.

HPLC was performed under the conditions described below.

### HPLC Analysis Conditions:

Column: Inertsil ODS-3 (250×4.6 mm) (available from GL Sciences, Inc.)
Eluent: water/acetonitrile/acetic acid=74/25/1
Flow rate: 1.0 mL/min
Column temperature: 40 °C
Detection: 305 nm

As a reference sample, urolithins available from DALTON PHARMA were dissolved in DMSO and used. As a result, urolithin M5 was obtained at a molar concentration of 450 μM.

### Experimental Example 19-1: Production of Urolithin A from Urolithin C

As a urolithin A precursor, urolithin C was added to an ABB culture medium (available from Oxoid Limited) at an amount to achieve a final concentration of 1 g/L, after which the mixture was heated and sterilized, the gas phase was purged with a gas of N₂:CO₂:H₂ (80%/10%/10%), and the resulting product was used as a basal culture medium. The basal culture medium was inoculated with the Clostridium bolteae JCM 12243 strain, the DSM 15670 strain, or the DSM 29485 strain and anaerobically cultured at 37 °C. After culturing was completed, urolithins were extracted from 5 mL of the culture solution using an equal amount of ethyl acetate, and the obtained ethyl acetate phase was concentrated under reduced pressure and dried to obtain a solid. The dry solid obtained in this manner was re-dissolved in 0.5 mL of methanol, and the amount of urolithins was quantitatively determined and analyzed through HPLC.

The HPLC conditions were as follows.

### <HPLC Conditions>

Column: Inertsil ODS-3 (4.6 mm inner diameter×250 mm length, 5 μm) (available from GL Sciences, Inc.)
Eluent A: 1% formic acid
   B: Acetonitrile containing 1% formic acid
Flow rate: 1 mL/min
Column temperature: 40 °C
Detection: UV (305 nm)

Also, as a reference sample, urolithins available from DALTON PHARMA were dissolved in DMSO and used.

As a result, two weeks of culturing resulted in the conversion of 89%, 100%, and 89% of the added urolithin C to urolithin A, respectively for each strain.

### Experimental Example 19-2: Production of Urolithin A from Urolithin C

Experimental Example 19-2 was conducted in the same manner as Experimental Example 19-1 with the exception that the Clostridium asparagiforme DSM 15981 strain was used and culturing was implemented for five days, and as a result, 95% of the added urolithin C was converted to urolithin A.

### Experimental Example 19-3: Production of Urolithin A from Urolithin C

Experimental Example 19-3 was conducted in the same manner as Experimental Example 19-1 with the exception that the Clostridium citroniae DSM 19261 strain was used and culturing was implemented for five days, and as a result, 82% of the added urolithin C was converted to urolithin A.

### Experimental Example 19-4: Production of Urolithin A from Urolithin C

An ABB culture medium (available from Oxoid Limited) containing 0.1% of ellagic acid (Sigma-Aldrich Corporation) was inoculated with the Clostridium bolteae JCM 12243 strain and the Gordonibacter pamelaeae DSM 19378 strain, and then cultured in the same manner as in Experimental Example 19-1, and as a result, after two weeks of culturing, 67% of the added ellagic acid was converted to urolithin A.

### Experimental Example 19-5: Production of Urolithin A from Urolithin C

Culturing was implemented in the same manner as in Experimental Example 19-4 with the exception that the Clostridium bolteae JCM 12243 strain and the Gordonibacter urolithinfaciens DSM 27213 strain were used, and as a result, after two weeks of culturing, 62% of the added ellagic acid was converted to urolithin A.

### Experimental Example 19-6: Production of Urolithin A from Urolithin C

Culturing was implemented in the same manner as in Experimental Example 19-4 with the exception that the Clostridium asparagiforme DSM 15981 strain and the Gordonibacter urolithinfaciens DSM 27213 strain were used, and as a result, after 5 days of culturing, 60% of the added ellagic acid was converted to urolithin A.

### Experimental Example 19-7: Production of Urolithin A from Urolithin C

Culturing was implemented in the same manner as in Experimental Example 19-4 with the exception that the Clostridium citroniae DSM 19261 strain and the Gordonibacter urolithinfaciens DSM 27213 strain were used, and as a result, after 5 days of culturing, 60% of the added ellagic acid was converted to urolithin A.

### Experimental Example 19-8: Production of Urolithin A from Urolithin C

Urolithin C was added as a substrate to an ABB culture medium (available from Oxide Limited), after which the mixture was heated and sterilized, the gas phase was purged with a gas of N₂:CO₂:H₂ (80%/10%/10%), and the resulting product was used as a culture medium. The culture medium containing urolithin C at a final concentration of 1.0 g/L was inoculated with the Clostridium sp. DC 3656 (NITE ABP-02708) strain and then anaerobically cultured at 37 °C for 5 days. After the culturing was completed, the culturing solution was analyzed by HPLC under the following conditions.

### <HPLC Conditions>

Column: Inertsil ODS-3 (4.6 mm inner diameter×250 mm length) (available from GL Sciences, Inc.)
Eluent: water/acetonitrile/acetic acid=74/25/1
Flow rate: 1.0 mL/min
Column temperature: 40 °C
Detection: UV (305 nm)

As a result, urolithin A was produced from urolithin C with a molar yield of 89%.

### Experimental Example 19-9: Production of Urolithin C from Ellagic Acid Using a Deposited Strain

Ellagic acid was added at a final concentration of 1 g/L to an ABB culture medium (available from Oxoid Limited), after which the mixture was heated and sterilized, the gas phase was purged with a gas of N₂:CO₂:H₂ (80%/10%/10%), and the resulting product was used as a basal culture medium. The basal culture medium was then inoculated with the Eggerthella sp. DC 3563 (NITE BP-02376) strain and anaerobically cultured for two weeks at 37 °C. The amount of urolithins in the culture solution after cultivation was quantitatively determined by the following method.

Urolithins were extracted from 5 mL of the culture solution with an equal amount of ethyl acetate, and the resulting ethyl acetate phase was concentrated under reduced pressure and dried to obtain a solid. The dry solid obtained in this manner was re-dissolved in 0.5 mL of methanol, and the amount of urolithins was quantitatively analyzed through HPLC.

HPLC was performed under the conditions described below.

### HPLC Analysis Conditions:

Column: Inertsil ODS-3 (250×4.6 mm) (available from GL Sciences, Inc.)
Eluent: water/acetonitrile/acetic acid=74/25/1
Flow rate: 1.0 mL/min
Column temperature: 40 °C
Detection: 305 nm

As a reference sample, urolithins available from DALTON PHARMA were dissolved in DMSO and used. As a result, urolithin C was obtained at a molar yield of 90.3% with respect to the added ellagic acid.

### Experimental Example 20: Measurement of Lactonase Activity

Genomic DNA derived from the Gordonibacter urolithinfaciens DSM 27213 strain was used as a template, and the lactonase gene was cloned by PCR using the following primer set and then inserted into pET-28a(+), and thereby an expression plasmid pET-28a_GuUroH-Histag (N) that can be expressed as a protein with His-tag added to the N-terminal side of lactonase was constructed.

(SEQ ID NO: 37)
5′-GCCGGATCCATGGCAGACAACAAGGTCATCGACATCAACAT
G-3′
(SEQ ID NO: 38)
5′-TATGAATTCCTACAGGTTGAACAGCTTCGCCGCGTTGCCGC
C-3′

The E. coli Rosetta2 (DE3) strain was transformed with pET-28a_GuUroH-Histag (N) through the Ca method.

The obtained transformed strain was shaken and cultured at 37 °C for 2.5 hours in an LB culture medium containing 5 mL of 34 μg/mL chloramphenicol and/or 30 μg/mL kanamycin. IPTG was added to the culture solution to a molar concentration of 0.1 mM and then subjected to induced culturing at 30 °C for 7 hours to obtain bacterial cells, and the bacterial cells thereof were then crushed, and a cell-free extract was prepared. The obtained cell-free extract was used to carry out a reaction for 7 hours at 37 °C, and the amount of urolithin M5 produced from ellagic acid, which was the raw material (substrate), was quantitatively determined through HPLC. The HPLC conditions were as follows.

### <HPLC Conditions>

Column: Cosmosil 5C18-AR-II (4.6 mm inner diameter×150 mm length)
Eluent A: 1% formic acid
   B: Acetonitrile containing 1% formic acid
Flow rate: 1 mL/min
Column temperature: 40 °C
Detection: UV (urolithin M5, 349 nm; urolithin M6, 348 nm; urolithin C, 337 nm)

As a result, the specific activity of the cell-free extract was 15.7 mU/mg-protein. Note that 1 U indicates the activity of catalyzing the production of 1 pmol of urolithin M5 in 1 minute under the conditions described above.

### Experimental Example 20-1: Construction of a Vector Expressing an Enzyme that Dehydroxylates the Hydroxyl Group at the 4-Position of Urolithins, and of a Transformed Strain

The Rhodococcus sp. expression vector pTipQC1 (Hokkaido System Science Co., Ltd.) was digested using the restriction enzymes EcoRI and BamHI, and linearized plasmid was obtained through gel extraction.

The primer sets described in Table 4 were used, and each gene fragment was amplified by PCR using, as a template, genomic DNA of the Gordonibacter urolithinfaciens DSM 27213 strain, and each inserted DNA fragment was obtained by gel extraction.

The linearized plasmid and each inserted DNA fragment were connected using NEBuilder HiFi DNA Assembly Mastermix (New England Biolabs Inc.).

TABLE 4
Primer Set

| Constructed Plasmid | Forward Primer | Reverse Primer |
|---|---|---|
| For pTIpQC1_uroA123 | | |
| uroA123: | uroA1_f (SEQ ID | uroA3_r (SEQ ID |
| | NO: 39) | NO: 42) |
| For pTIpQC1_uroA12 | | |
| uroA12: | uroA1_f (SEQ ID | uroA2_r (SEQ ID |
| | NO: 39) | NO: 41) |
| For pTIpQC1_uroA1 | | |
| uroA12: | uroA1_f (SEQ ID | uroA1_r (SEQ ID |
| | NO: 39) | NO: 40) |

The base sequences of the primers in the table are as follows.

uroA1_f:
(SEQ ID NO: 39)
5′-catcaccatatgggaattATGGCCGATTCAGCCCAGGCCCCCGTGC
AGGAG-3′
uroA1_r:
(SEQ ID NO: 40)
5′-agagatctaagcttgCTAGTCGGCCTTGCGGATCTTGCACATCAT
GGC-3′
uroA2_r:
(SEQ ID NO: 41)
5′-agagatctaagcttgCTACGCCTCGTTGATGGCGACGATCATCTGC
TT-3′
uroA3_r:
(SEQ ID NO: 42)
5′-agagatctaagcttgTTACCGGTAGATCGGTGTAACTTCGCGCTCC
GC-3′

The details of the constructed plasmids in the table are as follows.

pTIpQC1_uroA123 is a plasmid in which GuuroA1, GuuroA2, and GuuroA3 were inserted at the EcoR1 and BamHI sites of pTipQC1.

pTIpQC1_uroA12 is a plasmid in which GuuroA1 and GuuroA2 were inserted at the EcoR1 and BamHI sites of pTipQC1.

pTIpQC1_uroA1 is a plasmid in which GuuroA1 was inserted at the EcoR1 and BamHI sites of pTipQC1.

The vector was cloned using the E. coli DH5α strain as a host. Ampicillin was used as a selectable marker.

The produced plasmid was introduced into Rhodococcus erythropolis L88 (Hokkaido System Science Co., Ltd.) through electroporation. Chloramphenicol was used as the selectable marker.

The formed colony was cultured in an LB culture medium (20 μg/mL chloramphenicol) (28 °C), and a glycerol stock (30% glycerol) was prepared from the culture solution and stored (−80 °C), and then used to measure heterologous expression and enzyme activity.

### Experimental Example 20-2: Expression of Enzyme that Dehydroxylates the Hydroxyl Group at the 4-Position of Urolithins

An amount of 5 mL of an LB culture medium containing 20 μg/mL of chloramphenicol was added as a pre-culture to a 10 mL test tube, and then inoculated with the transformed strain and cultured while shaking at 300 rpm and 28 °C.

A 50 mL Erlenmeyer flask tube containing 23 mL of an LB culture medium containing 20 μg/mL of chloramphenicol was inoculated with 1 mL of the pre-culturing solution, and then cultured while shaking at 200 rpm and 28 °C for 7 hours, after which 0.1 μg/mL of thiostrepton was added to induce an enzyme for 16 hours.

Bacterial cells were collected by centrifugation (8,000 rpm, 10 min, 4 °C) and then washed twice with physiological saline to form dormant bacterial cells, which were then used in a reaction for measuring enzyme activity.

### Experimental Example 20-3: Measuring the Activity of an Enzyme that Dehydroxylates the Hydroxyl Group at the 4-Position of Urolithins

A reaction for the measuring activity was carried out under the following conditions.

A reaction solution containing 0.5 mg/mL of urolithin M5 or urolithin M6, 10 mM of reduced nicotinamide adenine dinucleotide phosphate (NADPH), 0.2 mM of flavin adenine dinucleotide (FAD), 10 mM of methyl viologen (MV), and 50 mM of a potassium phosphate buffer (pH 6.5) was reacted in AnaeroPack at 37 °C for 3 h while shaking at 200 spin.

An amount of 100 μL of the reaction solution was added to N,N-dimethylacetamide (DMA) containing 200 μL of 1% formic acid and mixed, after which the centrifuged supernatant was analyzed by HPLC, and the product was quantitatively determined.

The results are presented in Table 5. With the host L88 strain (R. erythropolis L88), urolithin M6 could not be detected, but the production of urolithin M6 was confirmed with the transformed strain (R. erythropolis_uroA123) containing GuuroA1, GuuroA2 and GuuroA3 and the transformed strain (R. erythropolis_uroA12) containing GuuroA1 and GuuroA2. In addition, with the transformed strain (R. erythropolis_uroA1) containing only GuuroA1, production of urolithin M6 could not be confirmed.

From these results, it was confirmed that at least GuuroA1 and GuuroA2 are required to produce urolithin M6 from urolithin M5. Also, with the host L88 strain, a host-derived protein may be complementary to the function of GuuroA3.

TABLE 5

| | Urolithin M5 (Residual | Urolithin M6 |
|---|---|---|
| Evaluated Strain | substrate) [mM] | (Product) [mM] |
| R. erythropolis L88 | 2.60 | Not detected |
| R. erythropolis_uroA123 | 1.85 | 0.90 |
| R. erythropolis_uroA12 | 2.42 | 0.29 |
| R. erythropolis_uroA1 | 2.36 | Not detected |

### Experimental Example 21-1: Construction of a Vector Expressing an Enzyme that Dehydroxylates the Hydroxyl Group at the 10-Position of Urolithins, and of a Transformed Strain

The Rhodococcus sp. expression vector pTipQC1 (Hokkaido System Science Co., Ltd.) was digested using the restriction enzymes EcoRI and BamHI, and linearized plasmid was obtained through gel extraction.

The primer sets described in Table 6 were used, and each gene fragment was amplified by PCR using, as a template, genomic DNA of the Gordonibacter urolithinfaciens DSM 27213 strain, and each inserted DNA fragment was obtained by gel extraction.

The linearized plasmid and each inserted DNA fragment were connected using NEBuillder HiFi DNA Assembly Mastermix (New England Biolabs Inc.).

TABLE 6
Primer Set

| Constructed Plasmid | Forward primer | Reverse primer |
|---|---|---|
| For pTipQC1_uroB123 | | |
| uroB123: | uroB1_f (SEQ ID | uroB3_r (SEQ ID |
| | NO: 43) | NO: 46) |
| For pTipQC1_uroB12 | | |
| uroB12: | uroB1_f (SEQ ID | uroB2_r (SEQ ID |
| | NO: 43) | NO: 45) |
| For pTipQC1_uroB1 | | |
| uroB1: | uroB1_f (SEQ ID | uroB1_r (SEQ ID |
| | NO: 43) | NO: 44) |

The primer sequences in the table are as follows.

uroB1_f:
(SEQ ID NO: 43)
5′-catcaccatatgggaattATGGTAGAGAACGCTGTCGCGGAGAAG
GACC-3′
uroB1_r:
(SEQ ID NO: 44)
5′-agagatctaagcttgTTACAGTTCCTCCTCCTTGCAGCGGTACA
CCTT-3′
uroB2_r:
(SEQ ID NO: 45)
5′-agagatctaagcttgCTACGCGCAGGGCGGCACCCACAGGTACT
GCTG-3′
uroB3_r:
(SEQ ID NO: 46)
5′-agagatctaagcttgCTAGCCCTTCTTCGCTGGCACGGGGTCCC
GCTC-3′

The details of the constructed plasmids in the table are as follows.

pTIpQC1_uroB123 is a plasmid in which GuuroB1, GuuroB2, and GuuroB3 were inserted at the EcoR1 and BamHI sites of pTipQC1.

pTIpQC1_uroB12 is a plasmid in which GuuroB1 and GuuroB2 were inserted at the EcoR1 and BamHI sites of pTipQC1.

pTIpQC1_uroB1 is a plasmid in which GuuroB1 was inserted at the EcoR1 and BamHI sites of pTipQC1.

The vector was cloned using the E. coli DH5α strain as a host. Ampicillin was used as a selectable marker.

The produced plasmid was introduced into Rhodococcus erythropolis L88 (Hokkaido System Science Co., Ltd.) through electroporation. Chloramphenicol was used as the selectable marker.

The formed colony was cultured in an LB culture medium (20 μg/mL chloramphenicol) (28 °C), and a glycerol stock (30% glycerol) was prepared from the culture solution and stored (−80 °C), and then used to measure heterologous expression and enzyme activity.

### Experimental Example 21-2: Expression of Enzyme that Dehydroxylates the Hydroxyl Group at the 10-Position of Urolithins

An amount of 5 mL of an LB culture medium containing 20 μg/mL of chloramphenicol was added as a pre-culture to a 10 mL test tube, and then inoculated with the transformed strain and cultured while shaking at 300 rpm and 28 °C for 2 to 3 days.

A 50 mL Erlenmeyer flask tube containing 25 mL of an LB culture medium containing 20 μg/mL of chloramphenicol was inoculated with 1% of the pre-culturing solution, and then cultured while shaking at 200 rpm and 28 °C for 7 hours, after which 0.2 μg/mL of thiostrepton was added to induce an enzyme for 48 hours.

Bacterial cells were collected by centrifugation (8,000 rpm, 10 min, 4 °C) and washed twice with a physiological saline solution to form dormant bacterial cells, which were then used in a reaction for measuring enzyme activity.

### Experimental Example 21-3: Measuring the Dehydroxylation Activity of Bacterial Cells that Dehydroxylate the Hydroxyl Group at the 10-Position of Urolithins

A reaction for the measuring activity was carried out under the following conditions.

A reaction solution containing 0.5 mg/mL of urolithin M6, 10 mM of methyl viologen (MV), 2 mg/mL of sodium hyposulfite, 2 mg/mL of sodium bicarbonate, and 50 mM of a potassium phosphate buffer (pH 6.5) was reacted in AnaeroPack at 37 °C for 12 h while shaking at 200 spin.

An amount of 100 μL of the reaction solution was added to N,N-dimethylacetamide (DMA) containing 200 μL of 1% formic acid and mixed, after which the centrifuged supernatant was analyzed by HPLC, and the product was quantitatively determined.

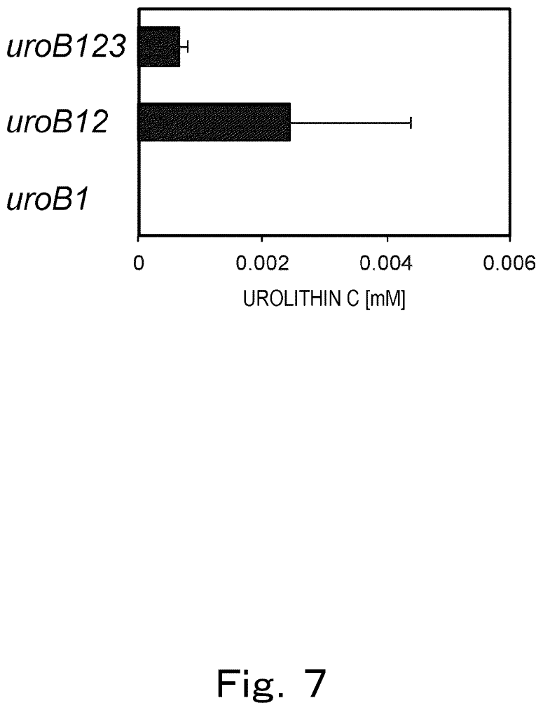

The results are presented in FIG. 7. With the host L88 strain (R. erythropolis L88), urolithin C could not be detected, but the production of urolithin M6 was confirmed with the transformed strain (R. erythropolis_uroB123, uroB123 in FIG. 7) containing GuuroB1, GuuroB2 and GuuroB3 and the transformed strain (R. erythropolis_uroB12, uroB12 in FIG. 7) containing GuuroB1 and GuuroB2. In addition, with the transformed strain (R. erythropolis_uroA1, uroB1 in FIG. 7) containing only GuuroB1, production of urolithin C could not be confirmed.

From these results, it was confirmed that at least GuuroB1 and GuuroB2 are required to produce urolithin C from urolithin M6. Also, with the host L88 strain, a host-derived protein may be complementary to the function of GuuroB3.

### Experimental Example 22-1: Production of an Enzyme that Dehydroxylates the Hydroxyl Group at the 10-Position of Urolithins

20 mL of a modified GAM culture medium (Nissui Pharmaceutical Co., Ltd.) containing 0.1 mg/mL of ellagic acid was inoculated with the Gordonibacter urolithinfaciens DSM 27213 strain and cultured at 37 °C for 5 to 6 days while shaking. In addition, 1.75 L of the same culture medium was inoculated with 10 mL of the obtained pre-culturing solution and then cultured at 37 °C for 7 to 9 days while shaking.

Bacterial cells were collected by centrifugation and washed twice with physiological saline, and washed bacterial cells were obtained.

Experimental Example 22-2: Partial purification of an enzyme that dehydroxylates the hydroxyl group at the 10-position of urolithins

Washed bacterial cells were suspended in a buffer and crushed by ultrasonic waves for 40 minutes.

A supernatant obtained by centrifuging (8,500 rpm, 30 minutes, 4 °C) the solution of crushed cells and then further subjecting to high-speed centrifuging (20,000 g, 60 min, 4 °C) was used as a cell-free extract.

The cell-free extract was then subjected to ultra-centrifugation (100,000 g, 110 min, 4 °C), and the supernatant thereby obtained was used as an ultra-centrifuged supernatant fraction. This ultra-centrifuged supernatant fraction was then used in an analysis of the following scientific properties of the enzyme.

### Experimental Example 23-1: Method for Measuring the Activity of the Enzyme that Dehydroxylates the Hydroxyl Group at the 10-Position of Urolithins

For the ultra-centrifuged supernatant fraction obtained in Experimental Example 22-2, the activity of dehydroxylating the hydroxyl group at the 10-position of urolithins was measured. Urolithin M6 was used as the urolithin having a hydroxyl group at the 10-position. When the hydroxyl group at the 10-position is dehydroxylated, urolithin C is produced.

An amount of 20 μL of a reaction solution for measuring enzyme activity was inserted into a 200 μL tube for PCR with the reaction solution containing 0.5 mg/mL of urolithin M6, 10 mM of NADPH (reduced nicotinamide adenine dinucleotide phosphate), 0.2 mM of FAD (flavin adenine dinucleotide), 10 mM of MV (methyl viologen), 50 mM of KPB (potassium phosphate buffer, pH 6.5) and a fraction obtained in Experimental Example 22-1. The gas phase was set to the gas phase in the anaerobic chamber (Vinyl Anaerobic Chamber available from Coy Labs), and the materials were reacted at 37 °C for 120 minutes. Next, after the reaction was completed, 40 μL of N,N-dimethylacetamide containing 1% formic acid was added to the reaction solution and mixed, after which the supernatant of centrifugation was analyzed by HPLC.

HPLC was carried out under the following conditions, and the resulting urolithin C was quantitatively determined (detection wavelength: 348 nm).

### <HPLC Conditions>

Column: COSMOSIL 5C18-ARII (4.6 mm inner diameter×150 mm length, available from Nacalai Tesque, Inc.)
Eluent: ultrapure water (Milli Q water)/acetonitrile (CH₃CN)/formic acid=80/20/1
Flow rate: 1.0 mL/min
Temperature: 40 °C
Detection: PDA (photodiode array) (The urolithin C was measured by UV absorption at 337 nm.)

The results of this experimental example correspond to the results listed for No. 9 in Table 7 for Experimental Example 23-2.

### Experimental Example 23-2: Effect of Cofactors on the Activity of Enzymes that Dehydroxylate the Hydroxyl Group at the 10-Position of Urolithins

The enzyme solution obtained in Experimental Example 22-2 was used, cofactors of the concentrations described in Table 7 were added to a reaction solution obtained by removing NADPH, FAD, and MV from the reaction solution for measuring enzyme activity described in Experimental Example 23-1, and the mixture was reacted, after which the produced urolithin C was quantitatively determined.

With the amount of urolithin C produced under the conditions indicated for No. 9 in Table 7 (10 mM of MV, 10 mM of NADPH, 0.2 mM of FAD) considered to be 100%, the amount of urolithin C produced under each condition was expressed as a relative activity. Note that in the following experimental examples, the conditions shown in No. 9 in Table 7 were used as standard activity measurement conditions.

### [Table 7]

TABLE 7

| | Cofactors (mM) | | | | | Relative |
|---|---|---|---|---|---|---|
| No. | NADH | NADPH | FAD | FMN | MV | Activity |
| 1 | — | — | — | — | — | 0.0% |
| 2 | — | — | — | — | 10 | 0.0% |
| 3 | 10 | — | — | — | 10 | 24.7% |
| 4 | — | 10 | — | — | 10 | 40.3% |
| 5 | — | — | 0.2 | — | 10 | 0.0% |
| 6 | — | — | — | 0.2 | 10 | 0.0% |
| 7 | 10 | — | 0.2 | — | 10 | 43.3% |
| 8 | 10 | — | — | 0.2 | 10 | 52.7% |
| 9 | — | 10 | 0.2 | — | 10 | 100% |
| 10 | — | 10 | — | 0.2 | 10 | 28% |

### Experimental Example 24: Temperature Dependency of Enzymes that Dehydroxylate the Hydroxyl Group at the 10-Position of Urolithins

Under the standard activity measurement conditions described in Experimental Example 23-2, the reaction temperature was changed, and the activity was measured.

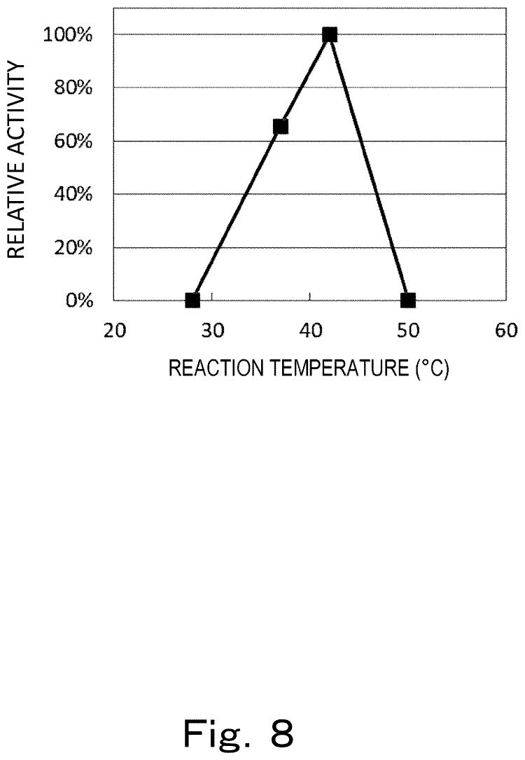

The results are illustrated in FIG. 8. The optimum temperature was 42 °C.

### Experimental Example 25: PH Dependency of Enzymes that Dehydroxylate the Hydroxyl Group at the 10-Position of Urolithins

Under the standard activity measurement conditions described in Experimental Example 23-2, the pH was changed using the following buffer, and the activity was measured.
Citric acid buffer: 50 mM citric acid-sodium citrate buffer
Acetic acid buffer: 50 mM acetic acid-sodium acetate buffer
Potassium phosphate buffer: 50 mM K₂HPO₄—KH₂PO₄ buffer
Tris-HCl buffer: 50 mM Tris-hydrochloride buffer
Carbonate buffer: 50 mM Na₂CO₃—NaHCO₃ buffer
Sodium phosphate buffer: 50 mM Na₃PO₄—Na₂HPO₄ buffer

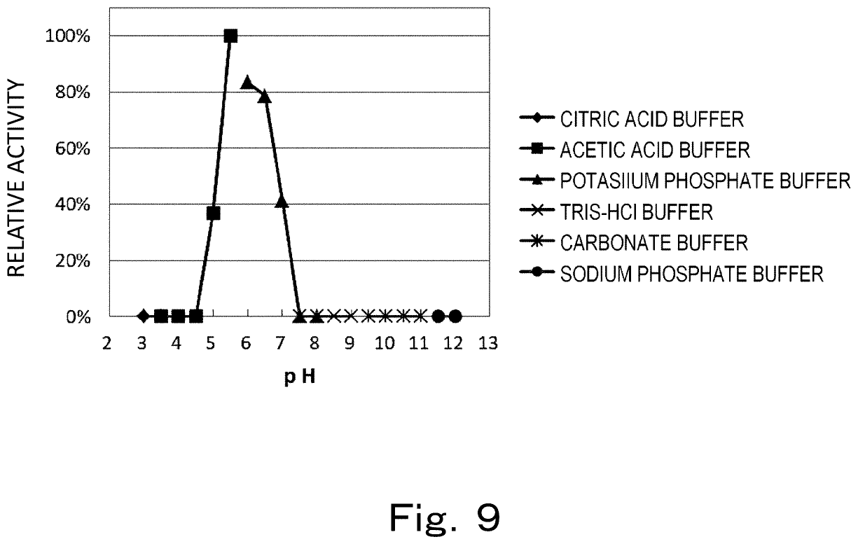

The results are illustrated in FIG. 9. The optimum pH was 5.5.

Experimental Example 26: Production of urolithin C from urolithin M5 using a deposited strain

Urolithin M5 was added to an ABB culture medium (available from Oxoid Limited) at an amount to achieve a final concentration of 3.3 mM, after which the mixture was heated and sterilized, the gas phase was purged with a gas of N₂:CO₂:H₂ (80%/10%/10%), and the resulting product was used as a basal culture medium. The basal culture medium was inoculated with the Gordonibacter urolithinfaciens DSM 27213 strain and anaerobically cultured at 37 °C. After culturing was completed, an equivalent amount of DMSO was added to 1 mL of the culturing solution to dissolve the urolithins, and the urolithins were subjected to a quantitative analysis by HPLC.

HPLC was implemented under the conditions described below.

### HPLC Conditions:

Column: Inertsil ODS-3 (φ4.6 mm×250 mm, 5 μm) (available from GL Sciences, Inc.)
Eluent A: 1% formic acid
   B: Acetonitrile containing 1% formic acid
Flow rate: 1 mL/min
Column temperature: 40 °C
Detection: UV (305 nm)

As a result, 0.0707 mM of urolithin C was produced by culturing for 14 days.

1-20. (canceled)

21. A recombinant vector for expression in a microorganism comprising a polynucleotide consisting essentially of:
(a) a first base sequence consisting of SEQ ID NO: 10, and a second base sequence consisting of SEQ ID NO: 11: or
(b) a first base sequence consisting of SEQ ID NO: 10, a second base sequence consisting of SEQ ID NO: 11, and a third base sequence consisting of SEQ ID NO: 12; or
(c) a fourth base sequence consisting of SEQ ID NO: 22, and a fifth base sequence consisting of SEQ ID NO: 23: or
(d) a fourth base sequence consisting of SEQ ID NO: 22, a fifth base sequence consisting of SEQ ID NO: 23, and a sixth base sequence consisting of SEQ ID NO: 24.

22. A microbial transformant,
(a) in which a polynucleotide is expressively retained,
wherein the polynucleotide consists essentially of:
(a-1) a first base sequence consisting of SEQ ID NO: 10, and a second base sequence consisting of SEQ ID NO: 11: or
(a-2) a first base sequence consisting of SEQ ID NO: 10, a second base sequence consisting of SEQ ID NO: 11, and a third base sequence consisting of SEQ ID NO: 12; or
(a-3) a fourth base sequence consisting of SEQ ID NO: 22, and a fifth base sequence consisting of SEQ ID NO: 23: or
(a-4) a fourth base sequence consisting of SEQ ID NO: 22, a fifth base sequence consisting of SEQ ID NO: 23, and a sixth base sequence consisting of SEQ ID NO: 24; or
(b) in which the vector according to claim 21 is expressively retained.

23. The microbial transformant according to claim 22, wherein the transformant is a transformed microorganism belonging to the genus Rhodococcus.

24-48. (canceled)

49. The recombinant vector according to claim 21, wherein the vector is expressed in a bacteria.

50. The microbial transformant according to claim 22, wherein the transformant is a transformed bacteria.

51. The recombinant vector according to claim 21, wherein the vector is expressed in a microorganism belonging to the genus Rhodococcus.


SEQUENCE LISTING (46 sequences)

SEQ ID NO: 1 - protein, 800 aa, Gordonibacter urolithinfaciens
MADSAQAPVQEHWSETNGKKYRRGSRLPKKTDTRPPVPAAETPKVPWTWEEDGMTVIRGTARSAPGCHNVCGILSYVKDGKLVKVEGDPEDPYNQGRLCSRCLCIPDYVYHEDRLTKPMKRDRADRGKDKFVEISWDEAYDIIVREFKRVVDTYGADKIAMCQGTGRDIHQVTRLNACVGSPNEGVPYFAGNSCYLPRIASMACMLGGACVMDCSQFLPQRYDDPRYTVPEYCIVWGHQPFYSNADGFYGHWITDLMKRGMKVAVVDPQLTWLAARAGEDWLRVRPGGDGALAMAMLKVVCDEKLYDQDFCDKWVYGLEAVCERVAEMDLDDLCERAWVAKEDVQRVARTYAASKPAAIQWGVALDQQTGGQQAAHAITALWCITGNLDVPGGNVMGDACWGIEQPNWTGTWGWDELMTEEEQSKRIGVERYPMFAVGFKNLSSNATIEAWQRGEIPIRAAYIVTNNFLATMGAQAAQQLEWYKQIDFIVVSDLFMTPTMMALADVVLPAATYEERDGFGGLNAYRISCINKAIEPVGDSKPDNTIFLELGKRLTAAIKPENQDIAWPWDDVQEMWDYALEDGGFTWDELRESTWKYPEFKYRKYETGDLRPDGQPGFRTETGRAEIYSMVFHHTSWSGLDPLPSYVEPVESPYSAPEDVEEYPYIVTSGARVPHFFHSEQRQVKKLRALHPDPLVYLHPDTAAANGIEEGDWVWLENKHGKCKYKAAFNDTYDPRVMQAEHGWWFPERKDAAEENVENEQEGLFGVLESNINNLVPFDAGVSGFGSNYKAMMCKIRKAD

SEQ ID NO: 2 - protein, 114 aa, Gordonibacter urolithinfaciens
MAYQGILVDVDYCTGCEACVLACQQEHGYTEAQFGLKITKLGPLHIDEAKKDYQYDFIPQFTKWCDLCAERVGKGKQPTCVQHCQAQCLDWGDVEDLAKKVDREKQMIVAINEA

SEQ ID NO: 3 - protein, 65 aa, Gordonibacter urolithinfaciens
MAQRFEDDFGGRIAEKLMNVDVHLTRDFSRSVTELEREQGRIDWKDRCGANMKKIAEREVTPIYR

SEQ ID NO: 4 - protein, 792 aa, Gordonibacter urolithinfaciens
MVENAVAEKDRDEIGTSKEENQFNFGAGKRQEVDPRWKHPKPFQYEEDGMVVTRTSVWSAPGCHEGCGVLVYSDKETGKFIKCEGDPDDPYNRGALCPRCLAFKQVEFHPDRILHPMKRAGERGSGQWERITWDEALETCYKEFRRISMTYGGDSIHCLRGTARDNQWQVGRMANTFGSPNEYGFLSGTACYLPRLSLMIMTYGGMLIADFSQFSALRYDDPEWVCPECTIVWGCNPTISNPDFFMGHWVTDAMKLGCKLISVEPRVTWLAAHADIHLQLRPGTDTGLALGMIKVIIDEDLYDHDFVERWTYGFDELAERARELSLDQVEAMTWVPKEKIIAAARLFATSKPANVVWGLAVDMQSQGTPCAAAIAALWTITGNLDVPGGMCYTASPMGVDQPSAGAWGIYDLINEEMQKKRVGWKEFPMYRYGLTQAMPDMCLEYMEEGKVKGVWIQTSNGIACMSCETERWYQAMKKPEFIAAVDIFMTPTIQSCADIVMPVQTWAEKHSVRAHYYFLSAITGGCAAEGEAKSDCEINRELAQYFDNDDEFNQAIGRPEGKQHTWPWATEDEVYDEIVSPSGFTFHELMEHGPVYQKYVYKKYEKGLMRPDGQPGFNTPTGRIEFYSTLFEKFGYDPLPYIEEPGIGPVTTPDLYEEYPLIMITGARTTSFFHSEHRQIPYLRQLTPDPWVQIHPRTAKDLNISEGDWVWIENHRGRCRQRARLTFEVHEKEIAAQHGWWFPEQDGAEPNLYGFRQSNINQLLANKPGTTGFGADLKCTLCKVYRCKEEEL

SEQ ID NO: 5 - protein, 119 aa, Gordonibacter urolithinfaciens
MALNGILVDYQYCTGCYSCEVACQAEHELPLEQWGVKVMQNGPWPVKDAEGNETDNYVYDFIPAFTKICDLCADRQEKGKLPSCVFHCQAKCMEFGPVDELAKRLDAKPQQYLWVPPCA

SEQ ID NO: 6 - protein, 186 aa, Gordonibacter urolithinfaciens
MKRRPAVDYQKLEEGIEKAPLASRPALERLLLYVSAGPDVSPDYAPYLEGSASYQDFFNAIYADDAQKGTSVWAEWAALKRKSWIGRFEPVLAVENLRLKGDGLPVQFGTGLFLAPTGSRDSIANLYVFERGAFNVEAAEFVTSIGGTFSCAGYDFAGIYGVYKYRGSVILEQWEAERDPVPAKKG

SEQ ID NO: 7 - DNA, 2403 bp, Gordonibacter urolithinfaciens
  source 1..2403: mol_type = genomic DNA
atggccgattcagcccaggcccccgtgcaggagcactggagcgagaccaacgggaagaagtaccgccgcggctcgcgcctgccgaagaagaccgacacgcgcccacccgtgcctgctgccgagacgccgaaagtgccttggacgtgggaggaggacggcatgaccgtcatccgcggcaccgcgcgcagcgcgcctggatgccacaacgtgtgcggcatcctgtcctacgtgaaggacggcaagctggtgaaagtggagggcgacccggaggacccctacaaccagggacgcctgtgcagccgctgcctctgcatccccgactacgtctaccatgaggaccgcctgaccaaaccgatgaagcgcgaccgcgccgaccgcggcaaggacaagttcgtggagatcagctgggacgaggcctacgacatcatcgtgcgcgagttcaagcgcgtcgtcgacacgtacggcgccgacaagatcgccatgtgccagggcacgggccgcgatatccaccaggtaacgcgcctgaacgcctgtgtgggctccccgaacgagggcgtgccctacttcgccggcaactcgtgctacctgccccgcatcgcgtcgatggcctgcatgctgggcggcgcctgcgtcatggactgctcgcagttcctcccccagcgctacgacgatccgcgctacacggtgccagagtactgcatcgtgtggggccaccaacccttctacagcaacgcagacggcttctacggccattggatcaccgacctcatgaagcgcggcatgaaggtggccgtggtggacccgcagctcacgtggctcgcggcccgcgccggcgaggactggctgcgcgtgcgtcccggcggcgacggcgcgctggccatggccatgctgaaggtcgtctgcgacgagaagctctacgaccaggacttctgcgacaagtgggtgtacggcctggaagccgtgtgcgagcgcgtggcggagatggacctggacgacctgtgcgagagggcctgggtggccaaggaggacgtgcagcgcgtggcgcgcacctacgccgcatcgaagcccgccgccatccaatggggcgtggcgctcgaccagcagacgggcggccagcaggcagcgcacgccatcacggccctgtggtgcatcaccggcaacctcgacgtgcccggcggcaacgtgatgggcgacgcctgctggggcatcgagcagccgaactggaccggcacctggggctgggacgagctcatgaccgaggaggagcagtccaagcgcatcggcgtggagcgctaccccatgttcgccgtgggcttcaagaacctgtcctccaacgccaccatcgaggcgtggcagcgcggcgagatacccatccgcgcggcctacatcgtgacgaacaacttcctggccaccatgggcgcccaggccgcccagcagctggaatggtacaagcagatcgacttcatcgtggtgtccgacctgttcatgacccccaccatgatggcgctggccgacgtggtgctgcccgccgccacctacgaggagcgcgacggcttcggcgggctcaacgcctaccgcatcagctgcatcaacaaggccatcgagcccgtgggcgactccaagcccgacaacaccatcttcctggagctgggcaagcgcctgacggcggccatcaagcccgagaaccaggacatcgcctggccttgggacgacgtgcaggagatgtgggactacgcccttgaggacggcggcttcacctgggacgagctgcgcgagtccacgtggaagtaccccgagttcaagtaccgcaagtacgagacgggagacctgcgccccgacggccagccgggcttccgcaccgagacgggccgcgcggagatctactccatggtgttccaccacacgtcgtggtcgggcctcgacccgctgccgagctacgtcgagccggtggagagcccctacagcgcccccgaggacgtggaggagtacccctacatcgtcacgtccggcgcgcgcgttccgcacttcttccactccgagcagcgccaggtcaagaagctgcgcgccctgcaccccgacccgctcgtgtacctgcaccccgacaccgcagccgccaacggaatcgaggagggcgactgggtgtggctggagaacaagcacggcaagtgcaagtacaaggccgccttcaacgacacctacgacccgcgcgtcatgcaggccgagcacggctggtggttccccgagcgcaaggacgcggccgaggagaacgtggagaacgagcaagagggcctgttcggcgtgctggagtccaacatcaacaacctcgtgcccttcgacgccggcgtatcgggcttcggctcgaactacaaggccatgatgtgcaagatccgcaaggccgactag

SEQ ID NO: 8 - DNA, 345 bp, Gordonibacter urolithinfaciens
  source 1..345: mol_type = genomic DNA
atggcttaccagggaattttggtcgacgtcgactactgcaccggctgcgaggcgtgcgtgctggcctgccagcaggaacacggctacaccgaggcgcagttcggcctgaagatcacgaagctgggcccgctgcacatcgacgaggccaagaaggactaccagtacgacttcatcccccagttcaccaagtggtgcgacctgtgcgccgagcgcgtgggcaagggcaagcagcccacgtgcgtgcagcactgccaggcccagtgcctggactggggagacgtggaggacctcgcgaagaaggtcgaccgcgagaagcagatgatcgtcgccatcaacgaggcgtag

SEQ ID NO: 9 - DNA, 198 bp, Gordonibacter urolithinfaciens
  source 1..198: mol_type = genomic DNA
atggcccagagattcgaggacgatttcggcgggaggatcgccgagaagctgatgaacgtcgacgttcacctgacgcgcgacttcagccgctcggtcaccgagctcgaacgcgagcagggccgcatcgactggaaggaccgctgcggcgccaacatgaagaagatcgcggagcgcgaagttacaccgatctaccggtaa

SEQ ID NO: 10 - DNA, 2379 bp, Gordonibacter urolithinfaciens
  source 1..2379: mol_type = genomic DNA
atggtagagaacgctgtcgcggagaaggaccgcgacgagatcggcacgtccaaggaggagaaccagttcaacttcggcgccggcaagaggcaggaagtggatcctcgctggaagcatccgaagcccttccagtacgaggaggacggcatggtggtcacccgcacctcggtgtggagcgcgccgggctgccacgagggctgtggcgtgctggtgtactccgacaaggagaccggcaagttcatcaagtgcgagggcgacccggacgacccgtacaaccgcggcgcgctgtgcccgaggtgcctggccttcaagcaggtggagttccaccccgaccgcatcctgcaccccatgaagcgtgccggcgagcgcgggagcggccagtgggagcgcatcacgtgggacgaggcgctggaaacctgctacaaggaattccgtcgcatctccatgacctacggcggcgactccatccactgcctgcgcggcaccgcacgcgacaaccagtggcaggtcggccgcatggcgaacacgttcggcagccccaacgagtacggcttcctgtccggcaccgcctgctacctgccccgcctgtcgctcatgatcatgacctacggcggcatgctcatcgccgacttctcgcagttctcggcgctgcgctacgacgaccccgagtgggtgtgccccgagtgcaccatcgtttggggctgcaaccccaccatctccaaccccgacttcttcatgggacactgggtcaccgacgccatgaagctcggctgcaagctcatctccgtcgagccgcgcgtcacctggctcgccgcgcacgccgacatccatctgcagctgcgccccggcaccgacacgggcctcgctctcggcatgatcaaggtcatcatcgacgaggacctctacgaccatgacttcgtggagcgctggacctacggcttcgacgagctggccgagcgcgcccgcgagctgtcgctggaccaggtggaggccatgacgtgggtccccaaggagaagatcatcgccgctgcgcgcctgttcgccacgtccaagccggccaacgtcgtgtggggcctcgccgtggacatgcagagccagggcacgccctgcgccgccgccatcgccgcgctgtggaccatcaccggcaacctcgacgtgccgggcggcatgtgctacaccgcctcccccatgggcgtcgaccagccgtccgccggcgcatggggcatctacgacctcatcaacgaggagatgcagaagaagcgcgtgggctggaaagaattccccatgtaccgctacggcctcacccaggccatgcccgacatgtgcctggagtatatggaggagggcaaggtcaagggcgtgtggatccagacatccaacggcattgcctgcatgagctgcgagaccgagcgctggtaccaggccatgaagaagccggagttcatcgccgccgtggacatcttcatgacgcctaccatccagtcgtgcgccgacatcgtcatgccggtgcagacgtgggccgagaagcactccgtacgcgcccactactacttcctgtccgctatcacgggcggctgcgcggccgagggcgaggcgaagtcggactgcgagatcaaccgcgagcttgcccagtacttcgacaacgacgacgagttcaaccaggccatcggccgccccgagggcaagcagcacacctggccgtgggcgaccgaggacgaggtgtacgacgagatcgtgagcccctccggcttcacgttccacgagctcatggagcacggcccggtctaccagaagtacgtgtacaagaagtacgagaagggcctcatgcgccccgacggccagcccggcttcaacacccccacgggccgcatcgagttctactccacgctgttcgagaagttcggctacgacccgctgccctacatcgaggagccgggcatcgggcccgtcaccacgcccgacctgtacgaggagtacccgctcatcatgatcacgggcgcccgcaccacgtcgttcttccactcggagcaccgccagatcccctacctgcgccagctcacacccgatccgtgggtgcagatccacccgcgcacggccaaggacctgaacatctccgagggcgactgggtctggatcgagaaccaccgcggccgctgccgccagcgtgcccgcctcacgttcgaggtgcacgagaaggagatcgccgcgcagcacggctggtggttccccgaacaggacggcgccgaacccaacctgtacggcttccgccagtccaacatcaaccagcttctggccaacaagcccggcacgactggcttcggtgccgacctgaagtgcacgctctgcaaggtgtaccgctgcaaggaggaggaactgtaa

SEQ ID NO: 11 - DNA, 360 bp, Gordonibacter urolithinfaciens
  source 1..360: mol_type = genomic DNA
atggctctcaacggaattctcgtcgactaccagtactgcacggggtgctactcgtgcgaggtggcctgccaggccgagcacgagctaccgctcgagcagtggggcgtgaaggtcatgcagaacggcccgtggccggtgaaggacgccgaaggcaacgagacggacaactacgtctacgacttcatcccggcgttcacgaagatctgcgatttgtgcgccgaccgccaggagaagggcaagctcccctcctgcgtgttccactgccaggccaagtgcatggagttcggcccggtggacgagctcgcgaagcgcttggacgccaagccgcagcagtacctgtgggtgccgccctgcgcgtag

SEQ ID NO: 12 - DNA, 561 bp, Gordonibacter urolithinfaciens
  source 1..561: mol_type = genomic DNA
ttgaaaaggagaccggctgtggactaccaaaagcttgaagagggcatcgagaaggcaccgctcgcctcgcgccccgcactggaacgcctgctgctgtacgtgtcggcaggcccggacgtatcgcccgactacgcgccgtacctggaaggctcggcatcctaccaggactttttcaacgccatctacgccgacgacgcccagaagggcacgtcggtatgggcggagtgggcggcgctcaaacgcaagagctggatcggccgcttcgaacccgtcctggccgtggagaacctgcgcctcaagggcgacgggctgccggtgcagttcggcacgggcctgttcctggcccctaccggcagccgcgacagcatcgcgaacctctacgtgttcgaacgcggcgcgttcaacgtggaggcggccgagttcgtcacgtcgatcggcggcacgttctcctgcgccggctacgacttcgccggcatctacggcgtgtacaagtaccgcggcagcgtgatactcgagcaatgggaggccgagcgggaccccgtgccagcgaagaagggctag

SEQ ID NO: 13 - protein, 800 aa, Gordonibacter pamelaeae
MADSAQAPVQEHWSETNGKKYRRGSRLPKKTDTRPPVPAAETPKVPWTWEEDGMTVIRGTARSAPGCHNVCGILSYVKDGKLVKVEGDPEDPYNQGRLCSRCLCIPNYVYHEDRLVKPMKRDRADRGKDKFVECSWDEAYDIIVREFKRVVDTYGADKIAMCQGTGRDIHQVTRLNACVGSPNEGVPYFAGNSCYLPRIASMACMLGGACVMDCSQFLPQRYDDPRYTVPKYCIVWGHQPFYSNADGFYGHWITDLMKRGMKVAVIDPQLTWLAARAGEDWLRVRPGGDGALAMAMLKVVCDEKLYDQEFCDKWVYGLEAVCERVAEMDLDDLCEKSWVAKEDVQRVARTYAASKPAAIQWGVALDQQTGGQQAAHAITALWCITGNLDVPGGNVMGDACWGIEQPNWTGTWGWDELMTEEEQSKRIGVERYPMFAVGFKNLSSNATIEAWQRGEVPIRAAYIVTNNFLATMGAQAEQQLEWYKQIDFIVVSDLFMTPTMMALADVVLPAATYEERDGFGGLNAYRISCINKAIEPVGDSKPDNTIFLELGKRLTAAIKPENQDIAWPWDDVQEMWDYALEDGGFTWDELRESTWKYPEFKYRKYETGDLRPDGQPGFRTETGRAEIYSMVFHHTSWSGLDPLPSYVEPVESPYSAPEDVEEYPYIVTSGARVPHFFHSEQRQIAKLRALHPDPLVYLHPDTAAANGIEEGDWVWLENKHGKCKYKAAFNDTYDPRVLQAEHGWWFPERKDAAEENVENEQEGLFGVLESNINNLVPFDAGVSGFGSNYKAMMCKIRKAD

SEQ ID NO: 14 - protein, 114 aa, Gordonibacter pamelaeae
MGYLGILVDVDYCTGCEACVLACQQEHGYTEREFGLKITKLGPLHIDEAKKDYQYDFIPQFTKWCDLCEERVGKGKQPTCVQHCQAQCLDWGRVEDLAKKVDREKQMIVAVKQA

SEQ ID NO: 15 - protein, 65 aa, Gordonibacter pamelaeae; identical to SEQ ID NO: 3

SEQ ID NO: 16 - protein, 792 aa, Gordonibacter pamelaeae
MVENAVAEKDRDEIGTSKEENQFNFGAGKRQEVDPRWKHPKPFQYEEDGMVVTRTSVWSAPGCHEGCGVLVYSDKETGKFIKCEGDPDDPYNRGALCPRCLAFKQVEFHPDRILHPMRRVGERGSGQWERITWDEALETCYKEFRRISMTYGGDSIHCLRGTARDNQWQVGRMANTFGSPNEYGFLSGTACYLPRLSLMIMTYGGMLIADFSQFSALRYDDPEWVCPECTIVWGCNPTISNPDFFMGHWVTDAMKLGCKLISVEPRVTWLAAHADIHLQLRPGTDTGLALGMIKVIIDEDLYDHDFVERWTYGFDELAERARELSLDQVEAMTWVPKEKIVAAARLFATSKPANVVWGLAVDMQSQGTPCAAAIAALWTITGNLDVPGGMCYTASPMGVDQPSAGAWGIYDLINEEMQKKRVGWKEFPMYRYGLTQAMPDMCLEYMEEGKVKGVWIQTSNGIACMSCETERWYQAMKKPEFIAAVDIFMTPTIQSCADIVMPVQTWAEKHSVRAHYYFLSAITGACAAEGEAKSDCEINRELAQYFDNDDEFNQAIGRPEGKQRTWPWATEDEVYDEIVSPSGFTFHELMEHGPVYQKYVYKKYEKGLMRPDGQPGFNTPTGRIEFYSTLFEKFGYDPLPYIEEPGIGPVTTPDLYEEYPLIMITGARATSFFHSEHRQIPYLRQLTPDPWVQIHPRTAKDLNISEGDWVWIENHRGRCRQRARLTFEVHEKEIAAQHGWWFPEQDGAEPNLYGFRQSNINQLLANKPGTTGFGADLKCTLCKVYRCKEEEL

SEQ ID NO: 17 - protein, 119 aa, Gordonibacter pamelaeae
MAMNGILVDYQYCTGCYSCEVACQAEHELPLEQWGVKVMQNGPWPVKDAEGNETDAYVYDFIPAFTKICDLCADRREKGKLPSCVFHCQAKCMEFGPVEELAKRLDAKPQQYLWVPPCA

SEQ ID NO: 18 - protein, 180 aa, Gordonibacter pamelaeae
MDYQELKEGIDQAPLASRATLERLLLYVSAGPGVSPDYAPYLEGAASYHDFFNAVYTDDAQKGTSVWAGWAALKRKSWIGRFEPDLAVENLRLKGDGLPVQFGTGLFLAPTGSRDNIANLYVFQRGAFNVEAAEFVTSIGGTFSCAGYDFAGIYGVYKYRGSVILEQWEAERAPVPTKKG

SEQ ID NO: 19 - DNA, 2403 bp, Gordonibacter pamelaeae
  source 1..2403: mol_type = genomic DNA
ctagtcggccttgcggatcttgcacatcatggccttgtagttcgagccgaagcccgacacgccggcgtcgaagggcacgaggttgttgatgttggactccaacacgccgaacaggccctcctgctcgttctccacgttctcctcggccgcgtctttgcgctcggggaaccaccagccgtgctcggcctgcaaaacgcgcgggtcgtaggtgtcgttgaaggcggccttgtacttgcacttgccgtgcttgttctccagccacacccagtcgccctcctcgatgccgttggcggccgcggtgtcgggatgcaggtacacgagcgggtcggggtgcagggcgcgcagcttcgcgatttggcgctgctcggagtggaagaagtgcggcacgcgggcgccggacgtgacgatgtaggggtactcctccacatcctcgggcgcgctgtaggggctctccaccggctccacgtagctcggcagcgggtcgaggcccgaccacgaggtgtggtggaacaccatggagtagatctccgcgcggcccgtctcggtgcggaagcccggctggccgtcggggcgcaggtcgcccgtctcgtacttgcggtacttgaactcggggtacttccacgtggactcgcgcagctcgtcccacgtgaagccgccgtcctccagcgcgtagtcccacatctcctgcacgtcgtcccagggccaggcgatgtcctggttctcgggcttgatggccgccgtcaggcgcttgcccagctccaggaagatggtgttgtcgggcttggagtcgcccacgggctcgatggccttgtttatgcagctgatgcggtaggcgttcagcccgccgaagccgtcgcgctcctcgtaggtggccgccggcagcaccacgtcggctagcgccatcatggtgggggtcatgaacaggtcggacaccacgatgaagtcgatctgcttgtaccattccagctgctgctccgcctgcgcgcccatggtggccaggaagttgttcgtcacgatgtaggccgcgcggatgggcacctcgccgcgctgccacgcctcgatggtggcgttggaggacaggttcttgaagcccacggcgaacatggggtagcgctccacgccgatgcgcttggactgctcctcctcggtcatgagctcgtcccagccccaggtgccggtccagttcggctgctcgatgccccagcacgcgtcgcccatgacgttgccgccgggcacgtcgaggttgccggtgatgcaccacagggccgtgatggcgtgcgcggcctgctggccgcccgtctgctggtccagcgccacgccccactggatggcggcgggcttcgacgcggcgtaggtgcgggcgacgcgctgcacgtcctccttggcaacccatgacttctcgcacagatcgtccaggtccatctcggccacgcgctcgcacacggcttccaggccgtagacccacttgtcgcagaactcctggtcgtagagcttctcgtcgcagacgaccttcagcatggccatggccagcgcgccgtcgccgccggggcgcacgcgtagccagtcctcgccggcgcgggccgcgagccacgtgagctgcgggtcaatcacggccaccttcatgccgcgcttcatgaggtcggtgatccagtggccgtagaagccgtcggcgttgctgtagaagggctggtggccccacacgatgcagtacttgggcaccgtgtagcgcgggtcgtcgtagcgctgggggaggaactgcgagcagtccatgacgcaggcgccgcccagcatgcaggccatcgacgcgatgcggggcaggtagcacgagttgccggcgaagtagggcacgccctcgttcgggctgcccacgcaggcgttcaggcgtgtcacctgatggatgtcgcggcccgtgccctggcacatggctatcttgtcggcgccgtaagtgtccacgacgcgcttgaactcgcgcacgatgatgtcgtaggcctcgtcccacgagcattccacgaacttgtccttgccgcggtcggcgcggtcgcgcttcatgggcttgaccaggcggtcctcatggtacacgtagttcgggatgcagaggcagcggctgcacaggcgcccctggttgtaggggtcctccgggtcgccctccaccttcaccagcttgccgtccttcacgtaggacaggatgccgcacacgttgtggcatcccggcgcgctgcgcgcggtgccgcggatcacggtcatgccgtcctcctcccacgtccaaggcaccttcggcgtctcggcggcgggcacgggcgggcgcgtgtcggtcttcttcggcaggcgcgagccgcggcggtacttcttcccgttggtctcgctccagtgctcctgcacgggtgcctgggctgaatcggccat

SEQ ID NO: 20 - DNA, 345 bp, Gordonibacter pamelaeae
  source 1..345: mol_type = genomic DNA
ctacgcctgcttgacggcgacgatcatctgcttctcgcggtcgaccttcttcgcgaggtcctccacacggccccagtccaggcactgggcctggcagtgctgcacgcaggtgggctgcttgcccttgcccacgcgctcttcgcacaaatcgcaccacttggtgaactgggggatgaagtcgtactggtagtccttcttggcttcgtcgatgtgcagcgggcccagcttcgtgatcttcaggccgaactcgcgctcggtgtagccgtgctcctgctggcaggccagcacgcacgcctcgcagccggtgcagtagtccacgtcgaccaaaattccaaggtatcccat

SEQ ID NO: 21 - DNA, 198 bp, Gordonibacter pamelaeae
  source 1..198: mol_type = genomic DNA
ttaccggtagatcggcgtgacttcgcgctccgcgatcttcttcatgttggcgccgcagcggtctttccagtcgatgcggccctgctcgcgctcgagctcggtgaccgagcggctgaagtcgcgcgtcaggtgaacgtcgacgttcatcagcttctcggcgatcctcccgccgaagtcgtcctcgaatctctgggccat

SEQ ID NO: 22 - DNA, 2379 bp, Gordonibacter pamelaeae
  source 1..2379: mol_type = genomic DNA
atggtagagaacgctgtcgcggagaaggaccgcgacgagatcggcacgtccaaggaggagaaccagttcaacttcggcgccggcaagaggcaggaggtggatccgcgctggaagcaccccaaacccttccagtacgaggaggacggcatggtggtcacccgcacctcggtgtggagcgcgccgggctgccacgagggctgcggcgtgctggtgtactccgacaaggagaccggcaagttcatcaagtgcgagggcgacccggacgacccgtacaaccgcggcgcgctgtgccccaggtgcctggccttcaagcaggtggagttccatcccgaccgcatcctgcaccccatgaggcgcgtgggcgagcgcggaagcggccagtgggagcgcatcacgtgggacgaggcgctggaaacctgctacaaggagttccgccgcatctccatgacctacggcggcgactccatccactgcctgcgcggcaccgcccgcgacaaccagtggcaggtcggccgcatggcgaacacgttcggcagccccaacgagtacggcttcctgtccggcaccgcctgctacctgccccgcctgtcgctcatgatcatgacctacggcggcatgctcatcgccgacttctcgcagttctcggcgctgcgctacgacgaccccgagtgggtgtgccccgagtgcaccatcgtctggggctgcaaccccaccatctccaaccccgacttcttcatgggccactgggtgaccgacgccatgaagctcggttgcaagctcatcagcgtggagccgcgcgtcacgtggctcgccgcgcacgccgacatccacctgcagctgcgccccggcaccgacacgggcctcgcgctcggcatgatcaaggtcatcatcgacgaggacctctacgaccacgacttcgtggagcgctggacctacggcttcgacgagctggccgagcgcgcccgcgagctgtcgctcgaccaggtggaggccatgacgtgggttcccaaggagaagatcgtggccgccgcgcgcctgttcgccacgtccaagccggccaacgtcgtgtggggactcgccgttgacatgcagagccagggcacgccctgcgccgccgccatcgcggcgctgtggaccatcaccggcaacctcgacgtgccgggcggcatgtgctacaccgcctcgcccatgggcgtggaccagccctccgccggcgcctggggcatctacgacctcatcaacgaggagatgcagaagaagcgcgtgggctggaaagagttccccatgtaccgctacggcctcacccaggccatgcccgacatgtgcctagagtacatggaggagggcaaggtcaagggcgtgtggatccagacgtccaacggcatcgcctgcatgagctgcgagaccgagcgctggtaccaggccatgaagaagcccgagttcatcgccgccgtggacatcttcatgacgcccaccatccagtcgtgcgccgacatcgtcatgccggtgcagacctgggccgagaagcactcggtacgcgcgcactactacttcctctccgccatcacgggcgcctgcgcggccgagggcgaggcgaagtcggactgcgagatcaaccgcgagctcgcgcagtacttcgacaacgacgacgagttcaaccaggccatcggccgccccgagggcaagcagcgcacctggccgtgggcgaccgaggacgaggtgtacgacgagatagtgagcccctccggcttcacgttccacgagctcatggagcatggcccggtctaccagaagtacgtgtacaagaagtacgagaagggcctcatgcgccccgacggtcagcccggcttcaacacccccaccggccgcatcgagttctactccacgctgttcgagaagttcggctacgacccgctgccctacatcgaggagccgggcatcgggcccgtcaccacgcccgacctctacgaggagtacccgctcatcatgatcacgggcgcccgcgccacgtcgttcttccactcggagcaccgccagatcccctacctgcgccagctcacgcccgacccgtgggtgcagatccacccgcgcacggcgaaggacctcaacatctccgagggcgactgggtgtggatcgagaaccaccgcggccgctgccgccagcgcgcccgcctcaccttcgaggtgcacgagaaggagatcgccgcacagcacggctggtggttccccgagcaggacggcgccgagccgaacctgtacggcttccgccagtccaacatcaaccagctgctcgccaacaagcccggcaccaccggcttcggcgccgatctgaagtgcacgctctgcaaggtgtaccgctgcaaggaggaggaactgtaa

SEQ ID NO: 23 - DNA, 360 bp, Gordonibacter pamelaeae
  source 1..360: mol_type = genomic DNA
ctacgcgcagggcggtacccacaggtactgctgcggcttggcgtccaggcgcttcgcgagctcttccaccgggccgaactccatgcacttggcctggcagtggaacacgcaggaggggagcttgcccttctcccggcggtcggcgcacagatcgcagatcttcgtgaacgcggggatgaagtcgtagacgtaagcatccgtctcgttgccctcggcgtccttcaccggccacgggccgttctgcatgaccttcacgccccactgttcgagcggcagctcgtgctcggcctggcaggccacctcgcacgagtagcaccccgtgcagtactggtagtcgacgagaattccgttcatggccat

SEQ ID NO: 24 - DNA, 543 bp, Gordonibacter pamelaeae
  source 1..543: mol_type = genomic DNA
ctagcccttcttcgtcggcacgggcgcgcgctcggcctcccattgctccagtatcacgctgccgcggtacttgtacacgccgtagatgccggcgaagtcgtagccggcgcaggagaacgtgccgccgatggacgtgacgaactcggccgcctcgacgttgaacgcgccgcgttggaacacgtagaggttcgcgatgttgtcgcggcttcccgtgggggccaggaacaggcccgtgccgaactgcaccggcagcccgtcgcccttgaggcgcaggttctccacggccaggtcgggctcgaagcgtccgatccagctctttcgcttgagcgcagcccaccccgcccacaccgacgtgcccttctgggcgtcgtcggtgtagacggcgttgaagaagtcatggtaggatgccgcgccttccaggtacggcgcgtaatcgggcgatacgcccgggcccgccgacacgtacagcagcaggcgctccagcgtggcacgcgaggcgagcggcgcctggtcgattccctctttcagctcttggtagtccat

SEQ ID NO: 25 - protein, 489 aa, Gordonibacter urolithinfaciens
MSNPNAAVAAREAKPVEKTPSYAWPCLITSVLAGIALVFAWQWLPGITFPVFKNWLAENNALFANPANFELMSNVMGLVPIGALIMALPASYLVRKIGPKAATVAGLALGAVGTAVSAVFVGDNFYAFLVGRFILGVALATTIVAGPTCVSVWFPDATRGRAMAIWSIWAPVGIFVINFIGNNVFEMAGSDMVAFQWIWVAVIVVFAVIFAIVFREPRENERSQVSPERKSFKEVLPFFKSRQLWCLILMFAIYNYVNYGFSQYLKTWMQTPELLGGLGWDAAAAGLWGGLICACGALAPIGGLILDKTPRDKKYLCVVVGILFLALASATFSQVTFFLPYVVFFCAGNMLLNACCRPLVPTLVFKGGATAVAFGLSFLTLGQYAGQMFTSYVMHPFSDTLTAASNVAIEAKQAVLASGGTPADFGPAIAQAAQEAAAAGIHVDPMLAVYALVPVSIVGILLAFGVKPSKKQAASAPAGKPADEAAAQH

SEQ ID NO: 26 - DNA, 1470 bp, Gordonibacter urolithinfaciens
  source 1..1470: mol_type = genomic DNA
atgagcaatccgaatgccgccgtagcggcaagggaagccaaacctgttgagaagacgccgtcgtacgcctggccatgcctcatcacgtcggtcctggccggcatcgcgctggtcttcgcctggcagtggctgccgggcatcacgttcccggtgttcaagaactggctggccgagaacaacgcgctgttcgccaacccggccaacttcgagctgatgtcgaacgtcatgggcctcgtgcctatcggcgcgctgatcatggccctgcccgcctcgtacctggtgcgcaagatcggcccgaaagccgcgaccgtcgccggcctcgcgctcggcgccgtcggcacggccgtgagcgccgtcttcgtgggcgacaacttctacgcgttcctcgtggggcgcttcattctgggcgtggccctggccaccaccatcgtcgcaggccccacctgcgtgtcggtgtggttccccgatgccacgcgcgggcgcgccatggccatctggtccatctgggcgcccgtgggcatcttcgtcatcaacttcatcggcaacaacgtgttcgagatggcgggcagcgacatggtggcgttccagtggatctgggtcgctgtcatcgtcgtgttcgccgtgatcttcgccatcgtgttccgcgagccgcgcgagaacgagcggtcgcaggtgtctcccgagcgcaagtccttcaaagaggtccttccgttcttcaagagccgccagctgtggtgcctgatcctcatgttcgccatctacaactacgtcaactacggcttcagccagtacctgaagacctggatgcaaacccccgagctgctgggcggcctgggctgggatgcggcggccgccggcctgtggggcggcctgatctgcgcgtgcggcgcgctggcccccatcggcggcttgatcctcgacaagacgcccagggataagaagtacctctgcgtcgtggtgggcatcctgttcctggcgctcgcatccgccacgttctcgcaggtcacgttcttcctcccctacgtcgtgttcttctgcgccggcaacatgctgctcaatgcctgctgccgcccgctcgtgccgacgctcgtgttcaagggcggcgccacggccgtggccttcggcttgtcgttcctgacgctgggccagtacgcgggccagatgttcacgagctacgtcatgcatccgttcagcgacacgctgaccgccgcctccaatgtggccatcgaggccaagcaggccgtgctcgcttccggcggcaccccggccgacttcggccccgccatcgcacaagcggcccaggaggccgccgccgctggtatccacgtcgaccctatgcttgccgtgtatgcgctcgtgcccgtgagcatcgtgggcatcctactcgccttcggcgtgaagccctctaagaagcaggccgcgagcgctcccgccggcaaacccgcagacgaggcagccgcacagcactaa

SEQ ID NO: 27 - protein, 514 aa, Gordonibacter pamelaeae
VRPSRHNPSTPCKEASDERGETMSNPAAATANGAPAAKPVEKTPSYAWPCLIVSLLAAVGIVFAWMWLPGITFPVFKNWLAENNAFFANPANFGLLSNVMGLVPIGALIMALPTTILVRKFGAKATTIIGVGCAVVGSAISAAFVGSNFYVFLAGRFILGLGLSTTVVSGPTCVSMWFPDATRGRAMAIWSCWAPVGIFLSNFVNDGVYHLVGSNMANLQWVWVAVAAVFGILFAVVFRDPRENERSQVSPERKPLKAVLKFFKSRQLWCLIIMFAIFNYMNYAFSQYLKTWLQTPEALGGFGWDATTAGLWGGLIVACGALAPLGGLILDKTPKSKKYLCVVVGILGLTICSALAFHAEFFIPYVIFFCIGNMMLNACCRPLVPTFVFKGGATAVAFGLSFLTLGQYAGQIVTSYALAPFNEGLTAASNVAINAKQAVLASGGTPADFGPAIGQAVQEAMAQGIHVDPMLAFWALVPIGVVGCLLAFGVKPSKKQAAGAPAAKPAEGEAAAQH

SEQ ID NO: 28 - DNA, 1545 bp, Gordonibacter pamelaeae
  source 1..1545: mol_type = genomic DNA
ttagtgttgcgcagccgcctcgccttcggcaggcttggcggcgggggcgccggcggcctgcttcttggacggtttcacgccgaacgcgagcaggcagcccacgacgccgatggggacgagtgcccagaaggcgagcatcggatccacatggatgccctgcgccatggcctcctgcacggcctggccaatggccgggccgaagtcggccggggtgccgccggaggcgagcacggcctgcttggcgttgatggccacgttggaggccgcggtgaggccctcgttgaagggcgcgagcgcgtagctcgttacgatctggccggcgtactggccgagcgtcaggaacgacaggccgaacgcgacggcggtagcgccgcccttgaacacgaacgtgggcacgagcgggcggcagcaggcattgagcatcatgttgccgatgcagaagaagatgacgtacgggatgaagaactcggcgtggaaggccaacgcagagcagatggtgagtcccaggatgcccacgacgacgcacaggtacttcttggatttcggcgttttgtcgaggatgaggccgccaagcggggccagcgctccgcaggcgacgatcaggccgccccaaaggccggcggtcgtggcatcccagccgaagccgccgagcgcttcgggcgtctgcagccacgtcttgaggtactggctgaacgcgtagttcatgtagttgaagatggcgaacatgataatgaggcaccacagttggcggctcttgaagaacttgagcacggccttgagcggcttgcgctcgggcgacacctgggagcgctcgttctcgcgcgggtcgcggaacacgacggcgaacaggatgccgaacacggcggccacggcgacccatacccactggaggttcgccatattggagcccaccagatggtacacgccgtcgttcacgaagttcgacaggaagattcccacgggcgcccagcacgaccaaatggccatggcgcgtccgcgcgtagcgtccgggaaccacatggaaacgcaggtgggaccggacacgacggtggtggacagacccaggcccagaatgaagcggccggccaggaacacgtagaagttgctgcccacgaacgccgcgctgatggcgctgccgacgaccgcgcagcccacgccgatgatcgtggtggccttcgcgccgaacttgcgcacgagaatggtggtgggcagcgccatgatcagcgcgccgatgggcacgagacccatgacgttggacagcaggccgaagttggctggatttgcgaagaacgcgttgttctcggccagccagttcttgaacaccgggaacgtgatgcccggaagccacatccacgcgaacacgatgcccacggccgccaagagcgacacgatgaggcacggccacgcgtaggatggcgtcttctccacgggcttggccgcgggtgcgccgttggctgtggcagcagcaggattgctcatagtttcccctctttcatcggacgcctccttgcaaggcgttgacggattgtgccgggaagggcgcac

SEQ ID NO: 29 - protein, 352 aa, Gordonibacter urolithinfaciens
MADNKVIDINMHFLPTDLFTNEEVLNGFLYSAPITMGLKAYRGKTPDGAKDQIVLATEDGKDILNYVEGDYTLETKLRAMDEVGVDIALLRMPVWQEWLPLDMCKIVNDQAAEMCRQSEGRLYANAVLPPWGRKEDIYELERCLGDLGMVGVQFACCYGDKFLDDELFKPYLKVLNDKKVPCAIHHTPGQNSFGNFADYTPLRRELGRIHVQATAVGREVYSGMFDEFPDLKFIHTMFGGNWFALKDLLAPHVSVKKKEAMNRLATNVDREAYDRYLANNIYFDMTHPMSWSKDQLELAVKTCGADHLLLGSSFPVFYEWMARSVGSVKALDVTQEEKDLMLGGNAAKLFNL

SEQ ID NO: 30 - DNA, 1059 bp, Gordonibacter urolithinfaciens
  source 1..1059: mol_type = genomic DNA
atggcagacaacaaggtcatcgacatcaacatgcacttcctgcccaccgacctgttcacgaacgaggaggtgctcaacggctttctgtactccgcccccatcaccatggggctcaaggcctaccgcggcaagacgcccgacggcgccaaggaccagatcgtgcttgccaccgaggacggcaaggacatcctgaactacgtggagggcgactacacgctggagacgaagctccgcgccatggacgaggtgggcgttgacatcgcgcttctgcgcatgccggtgtggcaggaatggctgccgctcgacatgtgcaagatcgtgaacgaccaggccgccgagatgtgccgtcagtcggaagggcgcctgtacgccaacgccgtgctgccgccgtggggccgcaaggaggatatctacgagctggagcgctgcctgggcgacctcggcatggtgggcgtgcaattcgcctgctgctacggcgacaagttcctcgacgacgagctgttcaagccctacctgaaggtgctcaacgacaagaaggtgccctgcgccatccaccatacgcccggacagaactcgttcggcaacttcgccgactacacgcccctgcgacgcgagctcggccgtatccacgtgcaggccaccgcagtgggccgcgaggtgtactcgggcatgttcgacgagttccccgacctcaagttcatccacacgatgttcggcggcaactggttcgcgctcaaggacctgctcgccccgcacgtgtcggtgaagaagaaggaggctatgaaccgcctggccaccaacgtcgaccgcgaggcgtacgaccgctacctagccaacaacatctacttcgacatgacgcaccccatgagctggagcaaggaccagctcgagctcgccgtgaagacgtgcggcgccgaccacctgcttctgggctcttcgttccccgtgttctacgagtggatggcccgctcggtgggctcggtgaaggccctcgacgtcacgcaggaggagaaggacctcatgctgggcggcaacgcggcgaagctgttcaacctgtag

SEQ ID NO: 31 - protein, 352 aa, Gordonibacter pamelaeae
MADNKVIDINMHFLPTDLFTNEEVLNGFLYSAPITMGLKAYRGKTPDGAKDQIVLATEDGKDILNYVEGDYTLETKLRAMDEVGVDIALLRMPVWQEWLPLDMCRIVNDQAAEMCRQSEGRLYANAVLPPWGRKEDIYELERCLGELGMVGVQFACCYGDKFLDDELFKPYLKVLNDKKVPCAIHHTPGQNAFGNFADYTPLRRELGRIHVQATAVGREVYSGMFDEFPDLKFIHTMFGGNWFALKDILAPHVSKQKKEAMNRLATNVDREAYDRYLANNIYFDMTHPMSWSKDQLELAVKTCGADHLLLGSSFPVFYEWMARSVGSVKALDVSQEEKDLMLGGNAAKLFNL

SEQ ID NO: 32 - DNA, 1059 bp, Gordonibacter pamelaeae
  source 1..1059: mol_type = genomic DNA
atggctgacaacaaggtcatcgacatcaacatgcacttcctgcccaccgacctgttcacgaacgaggaggtgctcaacggctttctgtactccgcgcccatcaccatggggctcaaggcctaccgcggcaagacgcccgacggcgccaaggaccagatcgtgctggccaccgaggacggcaaggacatcctgaactacgtggagggcgactacacgctggagacgaagctgcgcgccatggacgaggtgggcgttgacatcgcgcttctgcgcatgccggtgtggcaggaatggctgccgctcgacatgtgcaggatcgtgaacgaccaggccgccgagatgtgccgccagtcggaggggcgtctgtacgccaatgccgtgctgccgccgtggggccgcaaggaggacatctacgagctggagcgctgcctgggcgagctgggcatggtgggtgtgcagttcgcctgctgctacggcgacaagttcctcgacgacgagctgttcaagccctacctcaaagtgctcaacgacaagaaggtgccctgcgccatccaccacacgcccggccagaacgcgttcggcaacttcgccgactacacgccgctgcgccgcgagctcggccgcatccacgtgcaggccaccgcggtgggccgcgaggtgtactccggcatgttcgacgagttccccgacctcaagttcatccacacgatgttcggcggcaactggttcgcgctcaaggacattttggcaccgcacgtttcgaagcagaagaaggaggccatgaaccgcctggccaccaacgtcgaccgcgaggcgtacgaccgctatctcgccaacaacatctacttcgacatgacgcaccccatgagctggagcaaggaccagctggagctcgccgtgaagacgtgcggcgccgaccacctgctgctgggctcgtcgttcccggtgttctacgagtggatggcccgctcggtgggatcggtgaaggcgctcgacgtttcgcaggaggagaaggacctcatgctgggcggcaacgcggcgaagctgttcaacctgtag

SEQ ID NO: 33 - DNA, 42 bp, synthetic construct
  misc_feature 1..42: note = primer 33
  source 1..42: mol_type = other DNA
aaaggatccatgagcaatccgaatgccgccgtagcggcaagc

SEQ ID NO: 34 - DNA, 42 bp, synthetic construct
  misc_feature 1..42: note = primer 34
  source 1..42: mol_type = other DNA
gttgaattcttagtgctgtgcggctgcctcgtctgcgggttt

SEQ ID NO: 35 - DNA, 43 bp, synthetic construct
  misc_feature 1..43: note = primer 35
  source 1..43: mol_type = other DNA
gccggatcccatggcagacaacaaggtcatcgacatcaacatg

SEQ ID NO: 36 - DNA, 42 bp, synthetic construct
  misc_feature 1..42: note = primer 36
  source 1..42: mol_type = other DNA
tatgaattcctacaggttgaacagcttcgccgcgttgccgcc

SEQ ID NO: 37 - DNA, 42 bp, synthetic construct
  misc_feature 1..42: note = primer 37
  source 1..42: mol_type = other DNA
gccggatccatggcagacaacaaggtcatcgacatcaacatg

SEQ ID NO: 38 - DNA, 42 bp, synthetic construct; identical to SEQ ID NO: 36
  misc_feature 1..42: note = primer 38
  source 1..42: mol_type = other DNA

SEQ ID NO: 39 - DNA, 51 bp, synthetic construct
  misc_feature 1..51: note = uroA1_f
  source 1..51: mol_type = other DNA
catcaccatatgggaattatggccgattcagcccaggcccccgtgcaggag

SEQ ID NO: 40 - DNA, 48 bp, synthetic construct
  misc_feature 1..48: note = uroA1_r
  source 1..48: mol_type = other DNA
agagatctaagcttgctagtcggccttgcggatcttgcacatcatggc

SEQ ID NO: 41 - DNA, 48 bp, synthetic construct
  misc_feature 1..48: note = uroA2_r
  source 1..48: mol_type = other DNA
agagatctaagcttgctacgcctcgttgatggcgacgatcatctgctt

SEQ ID NO: 42 - DNA, 48 bp, synthetic construct
  misc_feature 1..48: note = uroA3_r
  source 1..48: mol_type = other DNA
agagatctaagcttgttaccggtagatcggtgtaacttcgcgctccgc

SEQ ID NO: 43 - DNA, 49 bp, synthetic construct
  misc_feature 1..49: note = uroB1_f
  source 1..49: mol_type = other DNA
catcaccatatgggaattatggtagagaacgctgtcgcggagaaggacc

SEQ ID NO: 44 - DNA, 48 bp, synthetic construct
  misc_feature 1..48: note = uroB1_r
  source 1..48: mol_type = other DNA
agagatctaagcttgttacagttcctcctccttgcagcggtacacctt

SEQ ID NO: 45 - DNA, 48 bp, synthetic construct
  misc_feature 1..48: note = uroB2_r
  source 1..48: mol_type = other DNA
agagatctaagcttgctacgcgcagggcggcacccacaggtactgctg

SEQ ID NO: 46 - DNA, 48 bp, synthetic construct
  misc_feature 1..48: note = uroB3_r
  source 1..48: mol_type = other DNA
agagatctaagcttgctagcccttcttcgctggcacggggtcccgctc